(19) United States
(12) Patent Application Publication
(10) Pub. No.: US 2010/0068301 A1
(43) Pub. Date: Mar. 18, 2010

(54) COMPOSITIONS AND TREATMENTS COMPRISING 5-LIPOXYGENASE-ACTIVATING PROTEIN INHIBITORS AND NITRIC OXIDE MODULATORS

(76) Inventors: John H. Hutchinson, La Jolla, CA (US); Mustapha Haddach, San Diego, CA (US); Mark Moran, Orinda, CA (US); Jillian Evans, San Diego, CA (US); Nicholas Simon Stock, San Diego, CA (US); Jeffrey Roger Roppe, Temecula, CA (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/517,166

(22) PCT Filed: Nov. 30, 2007
(86) PCT No.: PCT/US2007/086188

§ 371 Date: Jul. 14, 2009

Related U.S. Application Data
(60) Provisional application No. 60/868,024, filed on Nov. 30, 2006.

(51) Int. Cl. A61K 31/4709; A61K 31/404; C07D 401/12; A61K 33/26; A61K 31/04; A61K 31/34; A61K 31/195; A61K 31/7076; A61K 38/08; A61P 9/12

(52) U.S. Cl. 424/646; 514/314; 514/418; 546/174; 514/742; 514/470; 514/565; 514/46; 514/564; 514/563; 514/15; 514/361

(57) ABSTRACT

Disclosed herein are compositions and compounds that combine an inhibitor of 5-lipoxygenase activating protein (FLAP) and a modulator of NO levels in a mammal. The NO modulator can be an agent that induces the production of NO in a mammal, or can be an agent that itself produces NO in the mammal. Further disclosed herein are strategies for synthesizing such compounds, and methods for testing whether the combination compounds and compositions provide a desired benefit. Also disclosed herein are pharmaceutical compositions and formulations that combine a FLAP inhibitor and an NO modulator. Further described herein are methods for using such compositions and compounds for the treatment of diseases, conditions, and disorders in a mammal, including a human. Such treatment methods include the separate administration of a FLAP inhibitor and a NO modulator to the mammal, and the simultaneous administration of a FLAP inhibitor and a NO modulator to the mammal.

### RELATED APPLICATIONS

This application claims benefit of U.S. Provisional Application No. 60/868,024, entitled “COMPOSITIONS AND TREATMENTS COMPRISING 5-LIPOXYGENASE-ACTIVATING PROTEIN INHIBITORS AND NITRIC OXIDE MODULATORS” filed on Nov. 30, 2006, which is herein, incorporated by reference.

### FIELD OF THE INVENTION

The present disclosure relates to the field of medicine, specifically to methods and compositions for treating conditions with nitric oxide modulators in combination with, inhibitors of the leukotriene synthesis pathway, in particular, inhibitors of 5-lipoxygenase-activating protein.

### BACKGROUND OF THE INVENTION

Nitric oxide (NO) is a short-lived inorganic free radical that is an important biological second messenger. NO is biochemically synthesized from L-arginine and catalyzed by a family of enzymes called nitric oxide synthases. There are three distinct isoforms of the nitric oxide synthase (NOS) enzyme in mammals which share 51-57% homology with each other:
(i) a constitutive, Ca²⁺/calmodulin dependent enzyme, located in the endothelium, which releases NO in response to receptor or physical stimulation and is involved in the regulation of smooth muscle relaxation, the lowering of blood pressure, and inhibition of platelet aggregation (eNOS);
(ii) a constitutive, Ca²⁺/calmodulin dependent enzyme, predominantly found in neuronal tissue, which releases NO in response to receptor or physical stimulation and is important for long-term potentiation (nNOS); and
(iii) a Ca²⁺ independent enzyme which is induced after activation of vascular smooth muscle, macrophages, endothelial cells, and a number of other cells by endotoxin and cytokines (iNOS).

NOS is a complex enzyme that requires five cofactors for activity. The N-terminus is the oxygenase domain to which heme, tetrahydrobiopterin (H₄B), and L-arginine bind. The C-terminus reductase domain binds molecules FAD (and possibly FMN), and NADPH. See Huang, H. et al., J. Am. Chem. Soc., 123:2674-2676 (2001).

The protein 5-lipoxygenase activating protein (FLAP) is associated with the pathway of leukotriene synthesis. In particular, 5-lipoxygenase activating protein (FLAP) is responsible for binding arachidonic acid and transferring it to 5-lipoxygenase. See, e.g., Abramovitz, M. et al., Eur. J. Biochem. 215:105-111 (1993). 5-lipoxygenase can then catalyze the two-step oxygenation and dehydration of arachidonic acid, converting it into the intermediate compound 5-HPETE (5-hydroperoxyeicosatetraenoic acid), and in the presence of FLAP convert the 5-HPETE to LTA₄. Leukotrienes are biological compounds formed from arachidonic acid in the leukotriene synthesis pathway (Samuelsson et al, Science, 220, 568-575, 1983). They are synthesized primarily by eosinophils, neutrophils, mast cells, basophils, dendritic cells, macrophages and monocytes. Leukotrienes have been implicated in biological actions including, e.g., smooth muscle contraction, leukocyte activation, cytokine secretion, and vascular function.

### SUMMARY OF THE INVENTION

Described herein are compositions, methods, techniques and strategies comprising a combination of a first agent that can modulate, directly or indirectly, NO levels in an animal with a second agent that can modulate, directly or indirectly, the activity of 5-lipoxygenase-activating protein in an animal in regards to the first agent, modulation includes increasing the in vivo production of NO, decreasing inhibition of in vivo NO production, providing NO releasing agents, maintaining a level of NO, or decreasing the reduction of an NO level. In regards to the second agent, modulation includes antagonizing the activity of FLAP, inhibiting the activity of FLAP, decreasing the activity of FLAP, lowering the availability of arachidonic acid to FLAP, decreasing the transcription of FLAP, increasing the removal, degradation, or destruction of FLAP, and decreasing the translation of FLAP. Methods for use of such combinations include methods for treating a patient having a disease, disorder or condition in which inhibition of FLAP and modulation of NO provides therapeutic benefit. Examples of such diseases, disorders or conditions, include respiratory, cardiovascular, hypertensive, bone resorptive, malignant and inflammatory diseases, disorders and conditions,

The first agent can include (a) a moiety that can provide NO directly to a local site or systemically in the animal; (b) a moiety that upon chemical or enzymatic reaction can provide NO directly to a local site or systemically in the animal; (c) a moiety that upon irradiation can provide NO directly to a local site or systemically in the animal; (d) a moiety that can provide NO indirectly to a local site or systemically in the animal; (e) a moiety that upon chemical or enzymatic reaction can provide NO indirectly to a local site or systemically in the animal; (f) a moiety that upon irradiation can provide NO indirectly to a local site or systemically in the animal; (g) any of embodiments (a) through (f) wherein the moiety provides more than one NO relative to the second agent; (h) any of embodiments (a) through (f) wherein phrase “can provides NO directly to” is replaced with phrase “can modulate NO levels directly at”, or wherein the phrase “can provides NO indirectly to” is replaced with phrase “can modulate NO levels indirectly at”, in other words, modulation embodiments; (i) any of embodiments (a) through (h) wherein the first agent acts extracellularly; (j) any of embodiments (a) through (h) wherein the first agent acts intracellularly; (k) any of embodiments (a) through (h) wherein the first agent acts both extracellularly and intracellularly; (l) any of embodiments (a) through (k) wherein the second agent directly increases or promotes the activity of 5-lipoxygenase-activating protein; (m) any of embodiments (a) through (k) wherein the second agent indirectly increases or promotes the activity of 5-lipoxygenase-activating protein; (n) any of embodiments (a) through (k) wherein the second agent directly modulates the activity of 5-lipoxygenase-activating protein; (o) any of embodiments (a) through (k) wherein the second agent indirectly modulates the activity of 5-lipoxygenase-activating protein; (p) any of embodiments (a) through (k) wherein the second agent directly inhibits the activity of 5-lipoxygenase-activating protein; (q) any of embodiments (a) through (k) wherein the second agent indirectly inhibits the activity of 5-lipoxygenase-activating protein; (r) any of embodiments (l) through (q) wherein the second agent acts locally; (s) any of embodiments (l) through (q) wherein the second agent acts systemically; (t) any of embodiments (l) through (q) wherein the second agent acts intracellularly; (u) any of embodiments (l) through (q) wherein the second agent acts extracellularly; (v) any of embodiments (l) through (q) wherein the second agent sets intracellularly and extracellularly; (w) any of (a) through (v) wherein the first agent and the second agent act synergistically to provide, benefit, to the patient; (x) any of (a) through (v) wherein the activity of the first agent allows a lower therapeutically effective dose of the second agent relative to a therapeutically effective dose of the second agent administered in the absence of the first agent; (y) any of (a) through (v) wherein the activity of the second agent allows a lower therapeutically effective dose of the first agent relative to a therapeutically effective dose of the first agent administered in the absence of the second agent; or (z) any of (a) through (y) where first agent acts via modulates of a transcription factor.

The first agent includes a compound or complex, which upon administration to a patient in need, provides NO by chemically (including enzymatically) releasing a bound form of NO. Such, an agent can have 1, 2, 3, 4, 5, 6, 7, 8, 9, 10, or more bound NO groups that can be released chemically (including enzymatically). By way of example, a dimer, trimer, oligomer, multimer or polymer agent can include 2, 3 or more bound NO groups that can be released as desired, by means of a chemical (including enzymatic) reaction, further, the release of NO may occur via other means of activation, including photochemical, sonochemical, or electrochemical means of activation.

The two agents are either chemically linked or physically admixed; chemically linking includes covalent bonds, ionic bonds, hydrogen bonds, van der Waals interactions or a combination thereof. Chemical linking also includes a NO-modulator molecule connected to the FLAP inhibitor through a bridging (or linking) group, comprised of non-therapeutically active moieties or therapeutically active moieties, that can serve, as the chemical linkage, e.g., (FLAP inhibitor)-(bridging group)-(NO modulator). In the ease of an agent that releases NO in vivo, the agent would have inter alia vasodilatory properties and be anti-hypertensive. The first agent may increase or maintain NO levels in the animal. Further, such compositions may further be a component of a pharmaceutical composition. Also described herein are methods for treating a disease, disorder or condition in which the modulation of 5-lipoxygenase-activating protein favorably affects at least one symptom of a disease, disorder or condition. Such methods for treating comprise administration of a combination of a first agent that can modulate, directly or indirectly. NO levels in an animal with a second agent that can modulate, directly or indirectly, the activity of 5-lipoxygenase activating protein in an animal. Such a combination of agents includes chemically linked agents, physically admixed agents, or consecutive or simultaneous administration of physically separated agents.

Another aspect described herein are combination compounds of Formula (I), pharmaceutically acceptable salts, pharmaceutically acceptable N-oxides, pharmaceutically active metabolites, pharmaceutically acceptable prodrugs, and pharmaceutically acceptable solvates thereof, that are to antagonize or inhibit FLAP and/or modulate NO levels in vivo, and are used to treat patients suffering from NO-dependent, NO-mediated, leukotriene-dependent or leukotriene mediated conditions or diseases, including, but not limited to, hypertensive, asthma, myocardial infarction, cancer, and inflammatory conditions;

Aₓ-L-B Formula (I)

wherein,
A is a moiety mat in the form A′ (which includes A-X, A-H, A⁻, or A⁺) is an NO-modulator, or a moiety that upon activation/reaction produces NO, or a moiety selected from —NO₂ or —ONO₂, wherein X is COOH, CONH₂, OH, NH₂, halogen, SH, or CH₃; x is 0, 1, 2, 3, 4, 5, 6, 7, 8, 9, 10, 11, 12, 13, 14, 15, 16, 17, 17, 19, or 20; L is a bond or a moiety that chemically links the A and B moieties, and can be cleaved (in a single step or in multiple steps; chemically, enzymatically, biologically, photochemically, electrochemically, sonochemically, and by other activation means described herein) to produce L′-A and B′, L′-B and A′, or L′, A′ and B′; B is a moiety that in the form B′ (including B-X, B-H, B⁻, or B⁺) is a FLAP inhibitor.

L is attached to a suitable position on the FLAP inhibitor such that it is readily cleaved in vivo. For example, in the case where the FLAP inhibitor contains a carboxylic acid and L contains a hydroxy or amino group, L is coupled to the acid group of the FLAP inhibitor through an ester or amide bond, in the case that the FLAP inhibitor contains an amino group, L is attached through an amide, carbonate or carbamate group, in the case that the FLAP inhibitor contains an alcohol or phenol group, L is attached through an ether, ester, carbonate or carbamate group. In one aspect, L is a moiety that is cleaved in a single step or in multiple steps. In one embodiment, Aₓ-L-B is cleaved into a FLAP inhibitor and an NO modulator only or predominantly at a specific site in vivo; by way of example only, Aₓ-L-B remains intact upon administration, but is cleaved into a FLAP inhibitor and an NO modulator by a specific enzyme (e.g., a targeted esterase) that is produced only or predominantly by a particular cell type. In another embodiment, the FLAP inhibitor is released from Aₓ-L-B (or from L-B) by one site-specific pathway (single or multiple enzymes) and the NO modulator released from Aₓ-L-B (or Aₓ-L) by another site-specific pathway (single or multiple enzymes); alternatively, both the FLAP inhibitor and the NO modulator are released from Aₓ-L-B by the same site-specific pathway (single or multiple enzymes). In one aspect, a residue, or portion, of L remains with “A” or “B” after a cleavage step; in one aspect further cleavage steps remove further portions or residues of the L moiety still remaining with “A” or “B.” However, a residue or portion of “L” remains with “A” or “B” after cleavage or further reaction provided that the “A” group can act as an NO modulator and the “B” group can act as a FLAP inhibitor.

In one embodiment, if A or B includes a carboxylic acid group, then it is coupled to an L-B or L-A group via an ester or amide bond, in another embodiment, if A or B includes an amino group, then it is coupled to an L-B or L-A group via an amide, carbonate or carbamate bond. In yet another embodiment, if A or B contains an alkoxy or phenoxy moiety, then it is coupled to an L-B or L-A group via these moieties.

The present disclosure also provides compositions, methods, techniques and strategies comprising one or more NO modulators (including agents that release NO in a patient or agents that otherwise raise NO in a patient or, maintain production of NO or agents that otherwise maintain levels of NO in a patient) covalently linked to one or more FLAP inhibitors via a spacer moiety wherein a spacer allows the combination therapy to act in vivo. As used herein, the terms “release,” “raise,” “maintain,” “inhibit,” and “lower” refer to either a local effect, including an effect limited to a cell, a cell type, a disease site, an organ, and the like, or to a systemic, effect. In another embodiment, the spacer allows the release (including by way of example, a facilitated hydrolytic release) of one or more NO modulators (including agents that release NO in a patient or agents that otherwise raise NO in a patient or, alternatively, maintain production of NO or agents that otherwise maintain levels of NO in a patient) in vivo. In another embodiment, the spacer allows the release (including by way of example, a facilitated hydrolytic release) of one or more NO modulators (including agents that release NO in a patient or agents that otherwise raise NO in a patient or, alternatively, maintain production of NO or agents that otherwise maintain levels of NO in a patient) at an extracellular or intracellular site. Another embodiment allows the release (including by way of example, a facilitated hydrolytic release) of one or more FLAP inhibitors in vivo. Another embodiment allows the release (including by way of example, a facilitated hydrolytic release) of one or more FLAP inhibitors at an extracellular or intracellular site. In another embodiment allows the release (including by way of example, a facilitated hydrolytic release) of one or more NO modulators and one or more FLAP inhibitors at an extracellular or intracellular site.

Another embodiment described herein provides compositions, methods, techniques and strategies that allow or otherwise facilitate the enzymatic release of one or more NO modulators (including agents that release NO in a patient or agents that otherwise raise NO in a patient or, alternatively, maintain production of NO or agents that otherwise maintain levels of NO in a patient) in vivo. Another embodiment allows or otherwise facilitates site enzymatic release of one or more NO modulators (including agents that release NO in a patient or agents that otherwise raise NO in a patient or, alternatively, maintain production of NO or agents that otherwise maintain levels of NO in a patient) at an extracellular or intracellular site. Another embodiment allows or otherwise facilitates the enzymatic release of one or more FLAP inhibitors in vivo. Another embodiment allows or otherwise facilitates the enzymatic release of one or more FLAP inhibitors at an extracellular or intracellular site. Another embodiment allows or otherwise, facilitates the enzymatic release of one or more NO modulators (including agents that release NO in a patient or agents that otherwise raise NO in a patient or, alternatively, maintain production of NO or agents that otherwise maintain levels of NO in a patient) and one or more FLAP inhibitors at an extracellular or intracellular site.

In another embodiment described herein are compositions, methods, techniques and strategies comprising the release of one or more NO modulators (including agents that release NO in a patient or agents that otherwise raise NO in a patient or, alternatively, maintain production of NO or agents that otherwise maintain levels of NO in a patient) in vivo, facilitated by the administration of an additional agent. In another embodiment, the release of one or more NO modulators (including agents that release NO in a patient or agents that otherwise raise NO in a patient or, alternatively, maintain production of NO or agents that otherwise maintain levels of NO in a patient) at an extracellular or intracellular site is facilitated by the administration of an additional agent. In a further embodiment, the release of one or more FLAP inhibitors in vivo is facilitated by the administration of an additional agent. In a further embodiment, the release of one or more FLAP inhibitors at an extracellular or intracellular site is facilitated by the administration of an additional agent. Another embodiment facilitates the release of one or more NO modulators (including agents that release NO in a patient or agents that otherwise raise NO in a patient or, alternatively, maintain production of NO or agents that otherwise maintain levels of NO in a patient) and one or more FLAP inhibitors by the administration of an additional agent at an extracellular or intracellular site.

In another embodiment described herein are compositions, methods, techniques and strategies in which NO is delivered to a mammal exogenously. Alternatively, NO is stably attached to a molecule and this NO-conjugate molecule co-administered with a FLAP inhibitor, or the NO-conjugate molecule is chemically linked (including covalent bonds, ionic bonds, hydrogen bonds, van der Waals interactions or combinations thereof) directly to the FLAP inhibitor, or the NO-conjugate molecule is connected to the FLAP inhibitor through a bridging group, comprised of non-therapeutically active moieties, that cart serve as the chemical linkage i.e. (FLAP inhibitor)-(bridging group)-(NO modulator). In one embodiment, the chemically linked combination composition is cleaved in vivo to form two unlinked therapeutically active agents, in one embodiment, the NO-conjugate releases NO, which can activate plasma membrane-bound guanyl cyclase or cytoplasmic soluble guanyl cyclase and set in motion a number of pathways including a vasodilatory pathway, while the FLAP compound enters cells to inhibit synthesis of leukotrienes.

Another aspect of the present disclosure are pharmaceutical compositions comprising the NO modulators and/or FLAP modulators described herein (alone or in combination), and a pharmaceutically acceptable diluent, excipient, or carrier, in another aspect are methods for administering pharmaceutical compositions comprising the NO modulators and/or FLAP modulators described herein (alone or in combination), and a pharmaceutically acceptable diluent, excipient, or carrier. Such routes of administration include, by way of example, intravenous, oral, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

Another aspect of the present, disclosure are methods for treating a disease, disorder or condition in which the modulation, of FLAP favorably impacts at least one symptom of the disease, disorder or condition. Such methods comprise administration of first agent that can modulate, directly or indirectly, NO levels in an animal with a second agent that can modulate, directly or indirectly, the activity of 5-lipoxygenase-activating protein in an animal. Diseases, disorders or conditions that are treated using such a combination approach include respiratory, cardiovascular, hypertensive, NSAID-induced GI lesions, bone resorptive, malignant and inflammatory diseases, disorders and conditions.

Another aspect of the present disclosure are methods for treating a disease, disorder or condition in which the increase or maintenance of NO levels favorably impacts at least one symptom of the disease, disorder or condition. Such methods comprise administration of first agent that can modulate, directly or indirectly, NO levels in an animal with a second agent that can modulate, directly or indirectly, the activity of 5-lipoxygenase-activating protein in an animal. Diseases, disorders or conditions that are treated using such a combination approach include respiratory, cardiovascular, hypertensive, bone resorptive, malignant and inflammatory diseases, disorders and conditions.

In another aspect are methods for treating leukotriene-dependent or leukotriene mediated conditions or diseases, comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for treating inflammation comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for treating respirator diseases comprising administering to the mammal at least once an effective amount of at least one FLAP inhibitor and at least one NO modulator (including embodiments in which the FLAP inhibitor and NO modulator are administered as a single agent or as multiple agents). In a further embodiment of this aspect, the respiratory disease is asthma. In a further embodiment of this aspect, the respiratory disease includes, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, and seasonal asthma.

In another aspect are methods for preventing chronic obstructive pulmonary disease comprising administering to the mammal at least once an effective amount of at least one FLAP inhibitor and at least one NO modulator (including embodiments in which the FLAP inhibitor and NO modulator are administered as a single agent or as multiple agents). In a further embodiment of this aspect, chronic obstructive pulmonary disease includes, but is not limited to, chronic bronchitis or emphysema, pulmonary hypertension, interstitial lung fibrosis and/or airway inflammation and cystic fibrosis.

In another aspect are methods for preventing increased mucosal secretion and/or edema in a disease or condition comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for preventing or treating vasoconstriction, atherosclerosis and its sequalae myocardial ischemia, myocardial infarction, aortic aneurysm, vasculitis and stroke comprising administering to the mammal an effective amount of at least one FLAP inhibitor and at least one NO modulator (including embodiments in which die FLAP inhibitor and NO modulator are administered as a single agent or as multiple agents).

In another aspect are methods for reducing organ reperfusion injury following organ ischemia and/or endotoxic shock comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for reducing the constriction of blood vessels in a mammal comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for lowering or preventing an increase in blood pressure of a mammal comprising administering to the mammal at least once an effective amount of at least one FLAP inhibitor and at least one NO modulator (including embodiments in which fee FLAP inhibitor and NO modulator are administered as a single agent or as multiple agents).

In another aspect are methods for preventing eosinophil and/or basophil and/or dendritic cell and/or neutrophil and/or monocyte recruitment comprising administering to the mammal at least once an effective amount of at least one FLAP inhibitor and at least one NO modulator (including embodiments in which the FLAP inhibitor and NO modulator are administered as a single agent or as multiple agents).

A further aspect are methods for the prevention or treatment of abnormal bone remodeling, loss or gain, including diseases or conditions as, by way of example, osteopenia, osteoporosis, Paget's disease, cancer and other diseases comprising administering to the mammal at least once an effective amount of at least one FLAP inhibitor and at feast one NO modulator (including embodiments in which the FLAP inhibitor and NO modulator are administered as a single agent or as multiple agents).

In another aspect are methods for preventing ocular inflammation and allergic conjunctivitis, vernal keratoconjunctivitis, and papillary conjunctivitis comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for preventing CNS disorders comprising administering to the mammal at least once an effective amount of at least one FLAP inhibitor and at least one NO modulator (including embodiments in which the FLAP inhibitor and NO modulator are administered as a single agent or as multiple agents). CNS disorders include, but are not limited to, multiple sclerosis, Parkinson's disease, Alzheimer's disease, stroke, cerebral ischemia, retinal ischemia, post-surgical cognitive dysfunction, migraine, peripheral neuropathy/neuropathic pain, spinal cord injury, cerebral edema and head injury.

A further aspect are methods for the treatment of cancer comprising administering to the mammal at least once an effective amount of at least one FLAP inhibitor and at least one NO modulator (including embodiments in which the FLAP inhibitor and NO modulator are administered as a single agent or as multiple agents). The type of cancer may include, but is not limited to, pancreatic cancer and other solid or hematological tumors.

In another aspect are methods for preventing endotoxic shock and septic shock comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for preventing rheumatoid arthritis and osteoarthritis comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for preventing increased GI diseases comprising administering to the mammal at least once an effective amount of at least one FLAP inhibitor and at least one NO modulator (including embodiments in which the FLAP inhibitor and NO modulator are administered as a single agent or as multiple agents). Such, diseases include, by way of example only, chronic gastritis, eosinophilic gastroenteritis, and gastric motor dysfunction.

In another aspect are methods for preventing GI diseases comprising administering to the mammal at least once an effective amount of at least one FLAP inhibitor and at least one NO modulator (including embodiments in which the FLAP inhibitor and NO modulator are administered as a single agent or as multiple agents). Such diseases include, by way of example only, inflammatory bowel disease, ulcerative colitis and Crohn's disease.

A further aspect are methods for treating kidney diseases comprising administering to the mammal at least once an effective amount of at least one FLAP inhibitor and at least one NO modulator (including embodiments in which the FLAP inhibitor and NO modulator are administered as a single agent or as multiple agents). Such diseases include, by way of example only, glomerulonephritis, cyclosporine nephrotoxicity renal ischemia reperfusion.

In another aspect are methods for preventing or treating, acute or chronic renal insufficiency comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for treating type II diabetes comprising administering to the mammal at least once an effective amount of at least one FLAP inhibitor and at least one NO modulator (including embodiments in which the FLAP inhibitor and NO modulator are administered as a single agent or as multiple agents).

In another aspect are methods to diminish the inflammatory aspects of acute infections within one or more solid organs or tissues such as the kidney with acute pyelonephritis.

In another aspect are methods for preventing or treating acute or chronic disorders involving recruitment or activation of eosinophils comprising administering to the mammal at least once an effective amount of at least one FLAP inhibitor and at least one NO modulator (including embodiments in which the FLAP inhibitor and NO modulator are administered as a single agent or as multiple agents).

In another aspect are methods for preventing or treating acute or chronic erosive disease or motor dysfunction of the gastrointestinal tract caused by non-steroidal anti-inflammatory drugs (including selective or non-selective cyclooxygenase-1 or -2 inhibitors) comprising administering to the mammal at least once an effective amount of at least one FLAP inhibitor and at least one NO modulator (including embodiments in which the FLAP inhibitor and NO modulator are administered as a single agent or as multiple agents).

A further aspect are methods for the prevention or treatment of rejection or dysfunction in a transplanted organ or tissue comprising administering to the mammal at least once an effective amount of at least one FLAP inhibitor and at least one NO modulator (including embodiments in which the FLAP inhibitor and NO modulator are administered, as a single agent or as multiple agents).

In another aspect are methods for treating inflammatory responses of the skin comprising administering to the mammal at least once an effective amount of at least one FLAP inhibitor and at least one NO modulator (including embodiments in which the FLAP inhibitor and NO modulator are administered as a single agent or as multiple agents). Such inflammatory responses of the skin include, by way of example, dermatitis, contact dermatitis, eczema, urticaria, rosacea, and scarring. In another aspect are methods for reducing psoriatic lesions in the skin, joints, or other tissues or organs, comprising administering to the mammal an effective amount of at least one FLAP inhibitor and at least one NO modulator (including embodiments in which the FLAP inhibitor and NO modulator are administered as a single agent or as multiple agents).

A further aspect are methods for foe treatment of cystitis, including, by way of example only, interstitial cystitis, comprising administering to the mammal at least once an effective amount of at least one FLAP inhibitor and at least one NO modulator (including embodiments in which the FLAP inhibitor and NO modulator are administered as a single agent or as multiple agents).

A further aspect are methods for the treatment of metabolic syndromes such as Familial Mediterranean Fever comprising administering to the mammal at least once an effective amount of at least one FLAP inhibitor and at least one NO modulator (including embodiments in which the FLAP inhibitor and NO modulator are administered as a single agent or as multiple agents).

A flintier aspect are methods for the treatment of hypertension comprising administering to the mammal at least once an effective amount of at least one FLAP inhibitor and at least one NO modulator (including embodiments in which the FLAP inhibitor and NO modulator are administered as a single agent or as multiple agents).

A further aspect are methods for the improvement in the efficacy of antibiotic therapy in the setting of pulmonary or vascular infection or inflammation.

A further aspect are methods for the improvement in ventilation-perfusion matching in the lung in patients with pulmonary diseases or conditions.

The methods herein disclosed are also useful for preventing or reversing acute pulmonary vasoconstriction, such as may result from pneumonia, traumatic injury, aspiration or inhalation injury, rat embolism in the lung, acidosis, inflammation of the lung, adult respiratory distress syndrome, acute pulmonary edema, acute mountain sickness, asthma, post cardiac surgery acute pulmonary hypertension, persistent pulmonary hypertension of the newborn, perinatal aspiration syndrome, hyaline membrane disease, acute pulmonary thromboembolism, heparin-protamine reactions, sepsis, asthma, status asthmaticus, or hypoxia (including that which may occur during, one-lung anesthesia), as well as those cases of chronic pulmonary vasoconstriction which have a reversible component, such as may result from chronic pulmonary hypertension, bronchopulmonary dysplasia, chronic pulmonary thromboembolism, idiopathic or primary pulmonary hypertension, or chronic hypoxia.

A further aspect are methods for improvement in pulmonary function associated with further increases in exhaled NO when baseline levels of exhaled NO are abnormally high.

A further aspect are methods for the relaxation of smooth muscle cells that are abnormally contracted secondary to inflammation.

A further aspect are methods for the additive or synergistic enhancement of endothelial function, relaxation of abnormally contracted smooth muscles.

A further aspect are methods for the prevention or elimination of adverse effects from NO-supplementation to one type of cell by NO-supplementation to another type of cell.

A further aspect are methods for the prevention or reduction of adverse effects of NO-supplementation that are mediated or modulated by leukotrienes.

A further aspect are methods for the prevention or reduction of adverse effects of leukotriene inhibition that are mediated or modulated by nitric oxide supplementation.

A further aspect are methods for the treatment of erectile dysfunction comprising administering to the mammal at least once an effective amount of at least one FLAP inhibitor and at least one NO modulator (including embodiments in which the FLAP inhibitor and NO modulator are administered as a single agent or as multiple agents).

In a further aspect are methods to treat hepatorenal syndrome comprising administering to the mammal at least once an effective amount of at least one FLAP inhibitor and at least one NO modulator (including embodiments in which the FLAP inhibitor and NO modulator are administered as a single agent or as multiple agents).

In another aspect is the use of at least one FLAP inhibitor and at least one NO modulator (including embodiments in which the FLAP inhibitor and NO modulator are administered as a single agent or as multiple agents) in the manufacture of a medicament for treating an inflammatory disease or condition in an animal in which the activity of at least one leukotriene protein contributes to the pathology and/or symptoms of the disease or condition. In one embodiment of this aspect, the leukotriene pathway protein is 5-lipoxygenase-activating protein (FLAP). In another or further embodiment of this aspect, the inflammatory disease or conditions are respiratory, cardiovascular, or proliferative diseases.

In any of the aforementioned aspects are further embodiments in which administration is enteral, parenteral, or both, and wherein (a) the effective amount of the compound or compounds is systemically administered to the mammal; (b) the effective amount of the compound or compounds is administered orally to the mammal; (c) the effective amount of the compound or compounds is intravenously administered to the mammal; (d) the effective amount of the compound or compounds administered by inhalation; (e) the effective amount of the compound or compounds is administered by nasal administration; or (f) the effective amount of the compound or compounds is administered by injection to the mammal; (g) the effective amount of the compound or compounds is administered topically (dermal) to the mammal; (h) the effective amount of the compound or compounds is administered by ophthalmic administration; or (i) the effective amount of the compound or compounds is administered rectally to the mammal.

In any of the aforementioned aspects are further embodiments in which the mammal is a human, including embodiments wherein, (a) the human has an asthmatic condition or trait selected from the group consisting of allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational, asthma, steroid-resistant asthma, or seasonal asthma, or chronic obstructive pulmonary disease, or pulmonary hypertension or interstitial lung fibrosis. In any of the aforementioned aspects are further embodiments in which the mammal is an animal model for pulmonary inflammation, examples of which are provided herein.

In any of the aforementioned aspects are further embodiments comprising single administrations of the effective amount of the compound or compounds, including further embodiments in which (i) the compound or compounds is/are administered once; (ii) the compound or compounds is/are administered to the mammal multiple times over the span of one day; (iii) continually; or (iv) continuously.

In any of the aforementioned aspects are further embodiments comprising multiple administrations of the effective amount of the compound or compounds, including further embodiments in which (i) the compound or compounds is/are administered in a single dose; (is) the time between multiple administrations is every 6 hours; (iii) the compound or compounds is/are administered to She mammal every 8 hours. In further or alternative embodiments, the method comprises a drug holiday, wherein the administration of the compound or compounds is/are temporarily suspended or the dose of the compound or compounds being administered is/are temporarily reduced; at the end of the drug holiday, dosing of the compound or compounds is/are resumed. The length of the drug holiday can vary from 2 days to 1 year.

In one aspect, the NO modulator and the FLAP inhibitor are administered to a mammal as two separate agents. In a further aspect of this embodiment, the NO modulator and the FLAP inhibitor are administered to a mammal simulataneously. In another embodiment, the two agents are administered sequentially. In one aspect, the two agents are administered to the mammal in one pharmaceutical composition, in another aspect, the two agents are not in the same pharmaceutical composition.

In any of the aforementioned aspects involving the prevention or treatment of inflammation are further embodiments comprising: (a) monitoring inflammation in a mammal; (b) measuring bronchoconstriction in a mammal; (c) measuring eosinophil and/or basophil and/or dendritic cell and/or neutrophil and/or monocyte and/or lymphocyte recruitment in a mammal; (d) monitoring mucosal secretion in a mammal; (e) measuring mucosal edema in a mammal; (e) measuring levels of LTB₄ in the calcium ionophore-challenged blood of a mammal; (f) measuring levels of LTE₄ in the urinary excretion of a mammal; or (g) identifying a patient by measuring leukotriene-driven inflammatory biomarkers such as LTB₄, LTC₄, Il-6, CRP, SAA, MPO, EPO, MCP-1, MIP-α, sICAMs, Il-4, Il-13.

In any of the aforementioned aspects involving the prevention or treatment of leukotriene-dependent or leukotriene mediated diseases or conditions are further embodiments comprising identifying patients by screening for a leukotriene gene haplotype. In further or alternative embodiments the leukotriene gene haplotype is a leukotriene pathway gene, while in still further or alternative embodiments, the leukotriene gene haplotype is a 5-lipoxygenase-activating protein (FLAP) haplotype.

In any of the aforementioned aspects involving the prevention or treatment of leukotriene-dependent or leukotriene mediated diseases or conditions are further embodiments comprising identifying patients by monitoring the patient for either:
i) at least one leukotriene driven inflammatory biomarker; or
ii) at least one functional marker response to a leukotriene modifying agent; or
iii) at least one leukotriene driven inflammatory biomarker and at least one functional marker response to a leukotriene modifying agent.

In further or alternative embodiments, the leukotriene-driven inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In any of the aforementioned aspects involving the prevention or treatment of leukotriene-dependent or leukotriene mediated diseases or conditions are further embodiments comprising identifying patients by either:

i) screening the patient for at least one leukotriene gene haplotype; or

ii) monitoring the patient for at least one leukotriene driven inflammatory biomarker; or

ii) monitoring the patient for at least one functional marker response to a leukotriene modifying agent

In further or alternative embodiments, the leukotriene gene haplotype is a leukotriene pathway gene. In still further or alternative embodiments, the leukotriene gene haplotype is a 5-lipoxygenase-activating protein (FLAP) haplotype. In further or alternative embodiments, the leukotriene-driven inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In any of the aforementioned aspects involving the prevention or treatment of leukotriene-dependent or leukotriene mediated diseases or conditions are further embodiments comprising identifying patients by at least two of the following:

i) screening the patient for at least one leukotriene gene haplotype;

ii) monitoring the patient for at least one leukotriene driven inflammatory biomarker;

ii) monitoring the patient for at least one functional marker response to a leukotriene modifying agent.

In further or alternative embodiments, the leukotriene gene haplotype is a leukotriene pathway gene. In still further or alternative embodiments, the leukotriene gene haplotype is a 5-lipoxygenase-activating protein (FLAP) haplotype. In further or alternative embodiments, the leukotriene-driven inflammatory biomarkers are selected front the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In any of the aforementioned aspects involving the prevention or treatment of leukotriene-dependent or leukotriene mediated diseases or conditions are further embodiments comprising identifying patients by:

i) screening the patient for at least one leukotriene gene haplotype; and

ii) monitoring the patient for at least one leukotriene driven inflammatory biomarker; and

ii) monitoring the patient for at least one functional marker response to a leukotriene modifying agent.

In further or alternative embodiments, the leukotriene gene haplotype is a leukotriene pathway gene. In still further or alternative embodiments, the leukotriene gene haplotype is a 5-lipoxygenase-activating protein (FLAP) haplotype. In further or alternative embodiments, the leukotriene-driven inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1).

In another aspect is the prevention or treatment of leukotriene-dependent or leukotriene mediated diseases or conditions comprising administering to a patient an effective amount of a FLAP modulator, wherein the patients has been identified using information obtained by:

i) screening the patient for at least one leukotriene gene haplotype; and

ii) monitoring the patient for at least one leukotriene driven inflammatory biomarker; and

ii) monitoring the patient for at least one functional marker response to a leukotriene modifying agent.

In further or alternative embodiments, the FLAP modulator is a FLAP inhibitor. In further or alternative embodiments, the leukotriene gene haplotype is a leukotriene pathway gene. In still further or alternative embodiments, the leukotriene gene haplotype is a 5-lipoxygenase-activating protein (FLAP) haplotype. In further or alternative embodiments, the leukotriene-driven inflammatory biomarkers are selected from the group consisting of LTB₄, cysteinyl leukotrienes, CRP, SAA, MPO, EPO, MCP-1, MIP-α, sICAM, IL-6, IL-4, and IL-13, while in still further or alternative embodiments, the functional marker response is significant lung volume (FEV1). In further or alternative embodiments, the information obtained from the three diagnostic methods may be used in an algorithm in which the information is analyzed to identify patients in need of treatment with a FLAP modulator, the treatment regimen, and the type of FLAP modulator used.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description. All references cited herein, including patents, patent applications, and publications, are hereby incorporated by reference in their entirety.

### BRIEF DESCRIPTION OF THE FIGURES

FIG. 1 present illustrative methods for administration of the combination therapies disclosed herein.

FIG. 2 presents an illustrative dosage regimen of a combination therapy disclosed herein.

FIG. 3 presents illustrative methods for administration of the combination therapies disclosed herein.

FIG. 4 presents illustrative compositions according to Formula (I) that can be used in the combination therapies disclosed herein.

FIG. 5 presents illustrative compositions according to Formula (I) that can be used in the combination therapies disclosed herein.

FIG. 6 presents illustrative compositions according to Formula (I) that can be used in the combination therapies disclosed herein.

FIG. 7A shows the dose dependent inhibition of naproxen induced lesions using Compound 2-94 (filled circles) and isosorbide mononitrate (open circles).

FIG. 7B shows the inhibition of naproxen induced lesions using fractional doses of Compound 2-94 and isosorbide mononitrate.

FIG. 8 shows the maximum inhibition achieved by Compound 2-94 alone (30 mg/kg, striped bar), isosorbide mononitrate alone (100 mg/kg, open bar) and by Compound 2-94 plus isosorbide mononitrate as a co-treatment (10 mg/kg+30 mg/kg, filled bar).

### DETAILED DESCRIPTION OF THE INVENTION

The present disclosure provides compositions, methods, strategies and techniques for treating respiratory, hypertensive, cardiovascular, and inflammatory conditions by modulating nitric oxide levels and inhibiting FLAP. In one embodiment such compositions, methods, strategies and techniques comprise increasing vascular NO thereby decreasing hypertension, endothelial cell dysfunction and bone resorption, and inhibiting, at least in part, the activity of FLAP in a patient.

### Biological Aspects of NO

NO is involved in a number of biological actions in addition to endothelium-dependent relaxation including cytotoxicity of phagocytic cells and cell-to-cell communication in the central nervous system. Moncada, et al., Biochemical Pharmacology, 38:1709-1715 (1989). Nitroglycerin is one agent that can provide NO by conversion in the body to NO. Nitroglycerin has been administered to humans as a vasodilating agent in the treatment of cardiovascular disease, such as atherosclerosis. In atherosclerosis, plaque build up reduces the blood flow in the arteries. This in turn decreases oxygen supply to the heart muscle causing angina pectoris (chest pain) and sometimes even myocardial infarction. NO is used to treat the deprivation of blood circulation by stimulating membrane-bound or soluble guanylate cyclase. When NO is formed in the endothelial cell, it readily diffuses but of the cell and into adjacent smooth muscle cells where it binds to a heme moiety on guanylyl cyclase and activates the enzyme to produce cGMP from GTP. Increased cGMP activates a kinase that subsequently leads to the inhibition of calcium influx into the smooth muscle cell, which in turn decreases the smooth muscle tension development, causing vasodilation and consequently a decrease in blood pressure. The vasodilation of the blood vessels, leads to an increase in oxygen supply and thereby protects the heart from damage and cell death.

NO is an important signaling molecule outside the cardiovascular system. NO is involved in the signaling between nerve cells in the brain, is implicated in the normal defense against bacterial, and parasitic infections, and can be used to treat, high blood pressure. Alternatively, research indicates that elevated levels of NO can be pro-inflammatory. For example, NO may be biosynthesized from the induced isoform of NOS (iNOS) which is regulated transcriptionally and commonly induced by bacterial products and pro-inflammatory cytokines. As a result, the inflammatory diseases of the respiratory tract, such as asthma, are commonly characterized by art increased expression of iNOS within the respiratory epithelial and inflammatory immune cells, presumably as an additional host defense mechanism against bacterial or viral infections. A possible drawback of the overproduction of NO from iNOS is its accelerated metabolism to peroxynitrite. The formation of peroxynitrite and other reactive nitrogen species can contribute to the cause of inflammatory lung disease. See van der Vliet, A. et al., Respir. Res., 1:67-72 (2000).

iNOS vs eNOS

Inducible-nitric oxide synthetase and endothelial nitric oxide synthetase are both capable of producing NO. However, the cellular locations of these two enzymes differ: eNOS is found in endothelial cells and iNOS in (bronchial) epithelial cells, alveolar walls, and macrophages. (Kharitonov S A, “Influence of different therapeutic strategies on exhaled NO and lung inflammation in asthma and COPD,” Vascular Pharmacology, 2005 December; 43(6):371-8). Increases in exhaled NO have been considered to reflect only pulmonary pathology in the setting of bronchoconstriction. (Buchvald F. et al.) Exhaled nitric oxide predicts bronchoconstriction in asthmatic schoolchildren. (Chest, 2005:128:1964-7). Leukotriene responsiveness of tissues containing smooth muscle may vary as a function of whether the tissue is anatomically normal and whether leukotriene producing cells such as macrophages have immigrated. Such enhanced LT responsiveness has been, shown for atherosclerotic vessels, (Alien S, et al., Circulation, 1998; 97:2406-13.)

Therefore, benefit from NO supplementation, may be dependent upon which cell types are involved and whether the NO-induced effects are beneficially additive, synergistic or antagonistic. Heretofore the potential for interaction between NO-responsive cell types has not been emphasized. Moreover, the interpretation of baseline and changes in the level of exhaled NO may differ before and after NO supplementation. (Agvald P, et al., Vacul Pharmacol, 2005 at 14 Epub ahead of print).

### NO Modulators in Treatment

Many different modulators of NO are appropriate for the compositions, methods, strategies and techniques described herein. For example, nitrovasodilators, such as nitroprusside and nitroglycerine, inhibit vascular smooth muscle contractility to produce relaxation or to reduce vascular tone. Further, nitrovasodilators are classified as NO donors in part because they are metabolized at the sites of interests to release NO. See Moncada, et al. Eur J Clin Invest. August; 21(4):361-74 (1991); Moncada, et al. Pharmacol Rev. 1991 June; 43(2):109-42; Moncada, et al. Semin Perinatol. 1991 February; 15(1): 16-9. Other nitrovasodilators that modulate NO include, but are nor limited to, isosorbid mononitrate, and isosorbid dinitrate.

Suitable nitric oxide modulators include nitric oxide inducers, including compounds that stimulate endogenous NO or elevate levels of endogenous endothelium-derived relaxing factor (EDRF) in vivo or are substrates for nitric oxide synthase. Such compounds include, for example, L-arginine, L-homoarginine, and N-hydroxy-L-arginine, including their nitrosated and nitrosylated analogs (e.g., nitrosated L-arginine, nitrosylated L-arginine, nitrosated N-hydroxy-L-arginine, nitrosylated, N-hydroxy-L-arginine, nitrosated L-homoarginine and nitrosylated L-homoarginine), precursors of -arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid) and the substrates for nitric oxide synthase, cytokines, adenosine, bradykinin, calreticulin, bisacodyl, and phenolphthalein. EDRF is a vascular relaxing factor secreted by the endothelium, and has been identified as nitric oxide or a closely related derivative thereof (Palmer et al, Nature, 327:524-526 (1987); Ignarro et al, Proc. Natl. Acad. Sci. USA, 84:9265-9269 (1987)). Further compounds that may be employed to increase NO levels, include sodium nitroprusside; Molsidomine; 3-morpholinosydnonimine (SIN-1); 1,2,3,4-Oxatriazolium, 5-amino-3-(3,4-di-chlorophenyl)-chloride (GEA 3162); 1,2,3,4-Oxatriazolium, 5-amino-3-(3-chloro-2-methyl-phenyl)chloride (GEA502-4); 1,2,3,4-Oxatriazolium, 3-(3-chloro-2-methylphenyl)-5-[[[cyanomethylamino-]carbonyl]amino]-hydroxide inner salt (GEA 5583); S-nitroso-N-acetyl-D,L-penicillamine (SNAP); Glyco-SNAP-1, Glyco-SNAP-2,2,2′-(hydroxynitrosohydrazono)bis-ethanamine (NOC-18) and (+/−)-(E)-4-ethyl-2-[(E)-hydroxyimino]-5-nitro-3-hexe-namide (NOR-3); 1-[(4′,5′-Bis(carboxymethoxy)-2′-nitrophenyl)methoxy]-2-oxo-3,3-diethyl-1-triazene dipotassium salt (CNO-4); [1-(4′,5′-Bis(carboxymethoxy)-2′-nitrophenyl)methoxy]-2-oxo-3,3-diethyl-1-triazine diacetoxymethyl ester (CNO-5); nitroglycerin, diethylamine-NO (DEA/NO). IPA/NO, spermine-NO(SPER/NO), sulfite-NO (SULFI/NO), OXI/NO, and DETA/NO; cicletanine; sulfonamide NO donors GEA 3268, (1,2,3,4-oxatriazolium, 3-(3-chloro-2-methylphenyl)-5-[[(4-methoxyphenyl)-sulfonyl]amino]-, hydroxide inner salt) and GEA5145, (1,2,3,4-oxatriazolium, 3-(3-chloro-2-methylphenyl)-5-[(methylsulfonyl)amino]-, hydroxide inner salt); sulfonamide GEA 3175. Any of the aforementioned NO modulators may be used in the combination compositions and therapies described herein. In one embodiment, all statins are excluded as potential NO modulators.

### Pulmonary Hypertension

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hypertension is characterized by major structural changes in the pulmonary vasculature which result in a decreased cross-sectional area of the pulmonary blood vessels; this may be caused by, for example, chronic hypoxia, thromboembolism, or unknown causes (idiopathic or primary pulmonary hypertension).

Pulmonary hypertension has been implicated, in several life-threatening clinical conditions, such as adult respiratory distress syndrome (“ARDS”) and persistent pulmonary hypertension of the newborn (“PPHN”). (Zapol et al., Acute Respiratory Failure, p. 241-273, Marcel Dekker, New York (1985); Peckham, J. Ped. 93:1005 (1978).) PPHN, a disorder mat primarily affects full-term infants, is characterized by elevated pulmonary vascular resistance, pulmonary arterial hypertension, and right-to-left shunting of blood through, the patent ductus arteriosus and foramen ovale of the newborn's heart. Mortality rates range from 12-50%. (Fox, Pediatrics 59:205(1977); Dworetz, Pediatrics 84:1 (1989).) Pulmonary hypertension may also result in a potentially fatal heart condition known as “cor pulmonale”, or pulmonary heart disease. (Fishman, “Pulmonary Diseases and Disorders” 2nd Ed., McGraw-Hill, New York (1988).)

Attempts have been made to treat pulmonary hypertension by administering drugs with known systemic vasodilatory effects, such as nitroprusside, hydralazine, and calcium channel blockers. Although these drugs may be successful in lowering the pulmonary blood pressure, they typically exert an indiscriminate effect, decreasing not only pulmonary but also systemic blood pressure. A large decrease in the systemic vascular resistance may result in dangerous pooling of the blood in the venous circulation, peripheral hypotension (shock), right ventricular ischemia, and consequent heart failure. (Zapol (1985); Radermacher, Anaesthesiology 68:152 (1988); Vlahakes, Circulation 63:87 (1981).) For example, when intravenous nitroprusside was administered to 15 patients for treatment of acute pulmonary hypertension due to ARDS, mean PAP decreased from 29.6 to 24.2 mm Hg and pulmonary vascular resistance (PVR) decreased by a mean of 32%, but mean systemic arterial pressure was reduced from 89.6 mm Hg to the unacceptably low level of 70 mm Hg. (Zapol et al. 1985.) Intravenous nitroprusside was not recommended for clinical treatment of pulmonary hypertension, since it “markedly impairs pulmonary gas exchange by increasing Q_{VA}/Q_{T}” (the mixing of venous and arterial blood via an abnormal shunt). (Radermacher (1988).)

Physiological relaxation of blood vessels has been reported to result from the release of a very labile non-prostanoid endothelium-derived relaxing factor (EDRF) by endothelial cells lining the blood vessels. EDRF stimulates the enzyme guanylate cyclase within the vascular smooth muscle, with the resulting increase in cyclic GMP causing relaxation of this muscle, and thereby reversing vasoconstriction. Ignarro et. al., Proc. Natl. Acad. Sci. USA 84:9265 (1987) and Palmer et al., Nature 327:524 (1987) identified the vascular smooth muscle relaxation factor released by the endothelium of arteries and veins as nitric oxide (“NO”). NO is also believed to be produced by breakdown of organic nitrates such as nitroprusside and glyceryl trinitrate, (Ignarro, Circ. Res. 65:1 (1989); Furchgott, FASEB J. 3:2007 (1989).)

### Prevention or Amelioration of Adverse Effects Leukotriene-Inhibition Dependent Nitric Oxide Supplementation

Although NO supplementation has demonstrable beneficial effects in many settings, NO may be present in excess systematically, as manifested by (systemic) hypotension, or may be present in excess in certain tissues or organs (regional hypotension). NO overproduction and LTB₄ excess have been linked to adverse effects in the setting of hemorrhagic shock and resuscitation. (Kiang J G, Cell Res 2004; 14:450-9.)

In addition, although NO will relax smooth muscles in patients with systemic or pulmonary hypertension, or other vasoconstrictive disorders, NO can also worsen inflammation in arthritis (Reddy S V, Int Immunopharmacol 2005; 5:1085-90. Epub 2004 Dec. 15). Such an exacerbation may be mediated, in part, by leukotrienes.

Another benefit of therapeutic methods, formulations and compositions described herein, therefore, is the limitation of adverse effects of NO excess that occur when there is simultaneous LTB4 overproduction.

### Prevention or Amelioration of NO-Responsive Adverse Effects of Leukotriene Inhibition

The incidence of adverse effects associated with the pharmacological inhibition of leukotrienes is small and may not differ from that of placebo. (Antileukotriene Working Group, Ann Allergy Asthma Immunol., 2001:86(6 Suppl 1): 18-23) The incidence of dyspepsia with blockade of the cysteinyl leukotriene 1 receptor (2.1%) may be higher than that of placebo (1.1%). (PDR Electronic Library, 2005, Thomson.) NO supplementation is beneficial in conditions associated with dyspepsia. (Mourad, F H, et al., Eur J Gastroenterol Hepatol., 2000, 12(1):81-4.)

### Improvement in the Efficacy of Antibiotic Therapy

Nitric oxide deficiency may limit the effectiveness of antibiotics, particularly in a setting of infection and attendant inflammation. (Carlsson S, et al., Antimicrob Agents Chemother., 2005, 49(6):2352-5.) Moreover, abnormalities of both NO function and infection have been linked in the setting of atherosclerosis. (Bouwman J J et al., Eur J Clin Invest., 2005:35(9):573-82.)

### Inflammation-Induced Abnormal Contraction of Smooth Muscle

Inflammation suppresses intestinal motility in certain disorders. (Ozaki H, et al., Inflammopharmacology, 2005, 13:103-11.) In other conditions, hypermotility obtains (Physicians Desk Reference 2005.) When inflammation leads to abnormal smooth muscle contraction, an agent with combined anti-inflammatory activity and smooth muscle relaxant activity would be especially useful.

### NSAID-Induced Gastric Lesions/NSAID-Induced Gastric Injury

In one aspect, NO modulators and FLAP modulators are used for wound healing. In one aspect, NO modulators and FLAP modulators are used for the treatment of NSAID-induced gastric lesions. In yet another aspect are methods for preventing or treating NSAID-induced gastric lesions comprising administering to the mammal at least once an effective amount of an NO modulator, a FLAP inhibitor, or a compound of Formula (I).

### Biological Aspects of Leukotrienes

Leukotrienes (LTs) are potent contractile and inflammatory mediators produced by release of arachidonic acid from cell membranes and conversion to leukotrienes by the action of 5-lipoxygenase, 5-lipoxygenase-activating protein, LTA₄ hydrolase and LTC₄ synthase. The leukotriene synthesis pathway, or 5-lipoxygenase pathway, involves a series of enzymatic reactions in which arachidonic acid is converted to leukotriene LTB₄, or the cysteinyl leukotrienes, LTC₄, LTD₄, and LTE₄. The pathway occurs mainly at the nuclear envelope and has been described. See, e.g., Wood, J W et al, J. Exp. Med., 178:1935-1946, 1993; Peters-Golden, Am. J. Respir. Crit. Care Med. 157:8227-5232, 1998; Drazen, et al., ed. Five-Lipoxygenase Products in Asthma, Lung Biology in Health and Disease Series, Vol. 120, Chs. 1, 2, and 7, Marcel Dekker, Inc. NY, 1998. Protein components dedicated to the leukotriene synthesis pathway include a 5-lipoxygenase (5-LO), a 5-lipoxygenase-activating protein, a LTA₄ hydrolase, and a LTC₄ synthase. The synthesis of leukotrienes has been described in the literature, e.g., by Samuelsson et al, Science, 220, 568-575, 1983; Peters-Golden, Am J Respir Crit Care Med 157:S227-S232 (1998). Leukotrienes are synthesized directly from arachidonic acid by different cells including eosinophils, neutrophils, basophils, lymphocytes, macrophages, monocytes and mast cells. Excess LTA₄, for example from an activated neutrophil, may enter a cell by a transcellular pathway. Most cells in the body have LTA₄ hydrolase so can produce LTB₄. Platelets and endothelial, cells have LTC₄ synthase, so can make LTC₄ when presented with LTA₄ by a transcellular pathway.

Arachidonic acid is a polyunsaturated fatty acid and is present mainly in the membranes of the body's cells. Upon presentation of inflammatory stimuli from the exterior of the cell, calcium is released and binds to phospholipase A₂ (PLA2) mid 5-LO. Cell activation results in the translocation of PLA₂ and 5-LO from the cytoplasm, to the endoplasmic reticulum and/or nuclear membranes, where in the presence of FLAP, the released arachidonic acid is converted via a 5-HPETE intermediate to the epoxide LTA₄. Depending on the cell type, the LTA₄ may be immediately converted to LTC₄ by the nuclear-bound LTC₄ synthase or to LTB₄ by the action of cytosolic LTA₄ hydrolase. LTB₄ is exported from cells by an as yet uncharacterized transporter and may activate other cells, or the cell it was made in, via high affinity binding to one of two G protein-coupled receptors (GPCRs), namely BLT₁R or BLT₂R. LTC₄ is exported to the blood via the MRP-1 anion pump and rapidly converted to LTD₄ by the action of γ-glutamyl transpeptidase and LTD₄ is then converted to LTE₄ by the action of dipeptidases. LTC₄, LTD₄ and LTE₄ are collectively referred to as the cysteinyl leukotrienes (or previously as slow reacting substance of anaphylaxis, SRS-A). The cysteinyl leukotrienes activate other cells, or the cells they are made in, via high affinity binding to one of two GPCRs, namely CysLT₁R or CysLT₂R. CysLT₁ receptors are found in the human airway eosinophils, neutrophils, macrophages, mast cells, B-lymphocytes and smooth muscle and induce bronchoconstriction. Zhu et al. Am J Respir Cell Mol Biol Epub August 25 (2005). CysLT₂ receptors are located in human airway eosinophils, macrophages, mast cells the human pulmonary vasculature Figueroa et al, Clin Exp Allergy 33:1380-1388 (2003).

5-Lipoxygenase-activating protein has been shown to form two distinct multimeric complexes that regulate the formation of leukotrienes in RBL-2H3 cells: Mandal et al, PNAS, 103, 6587-6592 (2004). The first complex is the formation of homodimers or homotrimers of 5-lipoxygenase-activating protein, the second is the formation of heterodimers or heterotrimers involving 5-lipoxygenase-activating protein and LTC₄ synthase. The tight association of LTC₄ synthase with 5-lipoxygenase-activating protein and the low expression level of LTC₄ synthase imp lies that all the LTC₄ synthase is tied up in the heteromultimers with 5-lipoxygenase-activating protein. The formation of LTC₄ is likely regulated through the heterodimer or heterotrimer while the homodimer or homotrimer of 5-lipoxygenase-activating protein is responsible for the generation of LTA₄ that is then available for the conversion to LTB₄. Inhibition of 5-lipoxygenase results in the complete downstream inhibition of the formation of leukotrienes. In contrast, the existence of different multimeric complexes of 5-lipoxygenase-activating protein offers the possibility of differentially regulating the inhibition of the production of LTB₄ or the cysteinyl leukotrienes LTC₄, LTD₄ and LTE₄ through the preparation of 5-lipoxygenase-activating protein inhibitors selective for each multimeric complex. Thus, in one embodiment of the FLAP inhibitors described herein are FLAP inhibitors that are selective reach each multimeric complex of FLAP.

The involvement of leukotrienes in disease is described in the literature. See e.g., Busse, Clin. Exp. Allergy 26:868-79, 1996; O'Byrne, Chest 111(Supp. 2): 27S-34S, 1977; Sheftell, F. D., et al, Headache, 40:158-163, 2000; Klickstein et al., J. Clin. Invest, 66:1166-1170, 1950; and Davidson et al., Ann. Rheum. Dis., 42:677-679, 1983. Leukotrienes produce marked inflammatory responses in human skin. Evidence for the involvement of leukotrienes in a human disease is found in psoriasis, in which leukotrienes have been detected in psoriatic lesions (Kragballe et al., Arch. Dermatol., 119:548-552, 1983).

For example, inflammatory responses have been suggested to reflect three types of changes in the local blood vessels. The primary change is an increase in vascular diameter, which results in an increase in local blood flow and leads to an increased temperature, redness and a reduction in the velocity of blood flow, especially along the surfaces of small blood vessels. The second change is the activation of endothelial cells lining the blood vessel to express adhesion molecules that promote the binding of circulating leukocytes. The combination of slowed blood flow and induced adhesion molecules allows leukocytes to attach to the endothelium and migrate into the tissues, a process known as extravasation. These changes are initiated by cytokines and leukotrienes produced by activated macrophages. Once inflammation has begun, the first cells attracted to the site of infection are generally neutrophils. They are followed by monocytes, which differentiate into more tissue macrophages. In the latter stages of inflammation, other leukocytes, such as eosinophils and lymphocytes also enter the infected site. The third major change in the local blood vessels is an increase in vascular permeability. Instead of being rightly joined together, the endothelial cells lining the blood vessel walls become separated, leading to exit of fluid and proteins from the blood and their local accumulation in the tissue. (See Janeway, et al., Immunobiology: the immune system in health and disease, 5th ed., Garland Publishing, New York, 2001)

LTB₄ produces relatively weak contractions of isolated trachea and lung parenchyma, and these contractions are blocked in part by inhibitors of cyclooxygenase, suggesting that the contraction are secondary to the release of prostaglandins. However, LTB₄ has been shown to be a potent chemotactic agent for eosinophils and progenitors of mast cells and the LTB₄ receptor BLT1−/− knockout mouse is protected from eosinophilic inflammation and T-cell mediated allergic airway hyperreactivity. Miyahara et al., J Immunol 174:4979-4784; (Weller et al., J Exp Med 201:1961-1971(2005).

Leukotrienes C₄ and D₄ are potent smooth, muscle contractile agents, promoting bronchoconstriction in a variety of species, including humans (Dahlen et al., Nature, 288:484-486, 1980). These compounds have profound hemodynamic effects, constricting coronary blood vessels, and resulting in a reduction of cardiac output efficiency (Marone et al., in Biology of Leukotrienes, ed. By R. Levi and R. D. Krell, Ann. New York Acad. Sci. 524:321-333, 1988). Leukotrienes also act as vasoconstrictors, however, marked differences exist for different vascular beds. There are reports suggesting that leukotrienes contribute to cardiac reperfusion injury following myocardial ischemia (Barst and Mullane, Eur. J. Pharmacol., 114; 383-387, 1985; Sasaki et al., Cardiovasc. Res., 22:142-148, 1988), LTC₄ and LTD₄ directly increase vascular, permeability probably by promoting retraction of capillary endothelial cells via activation of the CysLT₂ receptor and possibly other as yet undefined CysLT receptors [Lotzer et al Arterioscler Thromb Vasc Biol 23: e32-36. (2003)]. LTB₄ enhances atherosclerotic progression in two atherosclerotic mouse models, namely low density receptor lipoprotein receptor deficient (LDLr−/−) and apolipoprotein E-deficient (ApoE−/−) mice (Aiello et al, Arterioscler Thromb Vasc Biol 22:443-449 (2002); Subbarao et al. Arterioscler Thromb Vasc Biol 24:369-375 (2004); Heller et al Circulation 112:578-586 (2005). LTB₄ has also been shown to increase human monocyte chemoattractant protein (MCP-1) a known enhancer of atherosclerotic progression (Huang et al Aterioscler Thromb Vasc Biol 24:1783-1788 (2004).

The role of FLAP in the leukotriene synthesis pathway is significant because FLAP in concert with 5-lipoxygenase performs the first step in the pathway for the synthesis of leukotrienes. Therefore the leukotriene synthesis pathway provides a number of targets for compounds useful in the treatment of leukotriene-dependent or leukotriene mediated diseases or conditions, including, by way of example, vascular and inflammatory disorders, proliferative diseases, and non-cancerous disorders.

Leukotriene-dependent or leukotriene mediated conditions treated using the methods, compounds, pharmaceutical compositions and medicaments described herein, include, but are not hunted to, bone diseases and disorder, cardiovascular diseases and disorders, inflammatory diseases and disorders, dermatological diseases and disorders, ocular diseases and disorders, cancer and other proliferative diseases and disorders, respiratory diseases and disorder, and non-cancerous disorders.

### FLAP Inhibition in Treatment

Leukotrienes are known to contribute to the inflammation of the airways of patients with asthma. CysLT₁ receptor antagonists such as montelukast (Singulair™) have been shown to be efficacious in asthma and allergic rhinitis [Reiss et al. Arch Intern Med 158:1213-1220 (1998); Phillip et al., Clin Exp Allergy 32:1020-1028 (2002)], CysLT₁R antagonists pranlukast (Onon™) and zafirlukast (Accolate™) have also been shown to be efficacious in asthma.

A number of drugs have been designed to inhibit leukotriene formation, including the 5-lipoxygenase inhibitor zileuton (Zyflo™) that has shown efficacy in asthma, Israel et al., Ann Intern Med 119:1059-1066 (1993), The 5-lipoxygenase inhibitor ZD2138 showed efficacy in inhibiting the fall of FEV1 resulting from aspirin-induced asthma, Nasser et al, Thorax, 49; 749-756 (1994). Other 5-lipoxygenase inhibitors include: CJ-13,610 (Mano et al, Chem. Pharm. Bull, 53, 965-973, 2005), ABT-761 (atreleuton; Stewart et al, J. Med Chem., 1997, 40, 1955-1968), AZD-4407 (European Patent EP 623614), L-739,010 (Hamel et al, J. Med. Chem., 40, 2866-2875, 1997), Wy-50,295 (Musser and Kreft, Drugs of the Future, 15, 73-80, 1990), TMK688 (Tohda et al, Clin. Exp. Allergy, 27, 110-118, 1997). See also Young, Eur. J. Med. Chem., 34, 671-685, 1999 and Werz Expert Opin. Ther. Patents, 15, 505-519, 2005. The following leukotriene synthesis inhibitors have shown efficacy in asthma: MK-0591, a specific inhibitor of 5-lipoxygenase-activating protein (FLAP), Brideau, et al., Ca. J. Physiol. Pharmacol. 70:799-807 (1992), MK-886, a specific inhibitor of 5-lipoxygenase-activating protein (FLAP), Friedman et al Am Rev Respir Dis. 147:839-844 (1993), and BAY X1005, a specific inhibitor of 5-lipoxygenase-activating protein (FLAP), Fructmann et al, Agents Actions 38:188-195 (1993).

FLAP inhibition will decrease LTB₄ from monocytes, neutrophils and other cells involved in vascular inflammation and thereby decrease atherosclerotic progression. The FLAP inhibitor MK-886 has been shown to to decrease the postangioplasty vasoconstrictive response in a porcine carotid injury model Provost et al., Brit J Pharmacol 123:251-258 (1998). MK-886 has also been shown to suppress femoral artery intimal hyperplasia in a rat photochemical model of endothelial injury Kondo et al., Thromb Haemost 79:635-639 (1998). The 5-lipoxygenase inhibitor zileuton has been shown to reduce renal ischemia in a mouse model, Nimesh et al. Mol Pharm 66:220-227 (2004).

FLAP modulators have been used for the treatment of a variety of diseases or conditions, including, by way of example only, (i) inflammation (see e.g. Leff A R et al., Ann Allergy Asthma Immunol 2001; 86 (Suppl 1)4-8; Riccioni G, et al., Ann Clin Lab Sci. 2004, 34(4):379-870; (ii) respiratory diseases including asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma (see e.g. Riccioni et al, Am. Clin. Lab. Sci., v34, 379-387 (2004)); (iii) chronic obstructive pulmonary disease, including chronic bronchitis or emphysema, interstitial lung fibrosis and/or airway inflammation and cystic fibrosis (see e.g. Kostikas K et al., Chest 2004; 127:1553-9); (iv) increased mucosal secretion and/or edema in a disease or condition (see e.g. Shahab R et al, J Laryngol Otol., 2004; 118:500-7); (v) vasoconstriction, atherosclerosis and its sequalae myocardial ischemia, myocardial infarction, aortic aneurysm, vasculitis and stroke (see e.g. Jala et al, Trends in Immunol., v25, 315-322 (2004) and Mehrabian et al, Curr. Opin. Lipidol, v14, 447-45 (2003)); (vi) reducing organ reperfusion injury following organ ischemia and/or endotoxic shock (see e.g. Matsui N, Fukuishi N, Fukuyama Y, Yasui Y, Akagi M., “Protective effect of the 5-lipoxygenase inhibitor ardisiaquinone A on hepatic ischemia-reperfusion injury in rats”, Planta Med. 2005 August; 71(8): 17-20); (vii) reducing the constriction of blood vessels (see e.g. Stanke-Labesque F et al., Br J Pharmacol. 2003 September; 140(1): 186-94); (viii) lowering or preventing an increase in blood pressure (see e.g. Stanke-Labesque F et al., Br J Pharmacol 2003 September; 140(1): 186-94, and Walch L, et al., Br J Pharmacol. 2002 December; 137(8): 1339-45): (ix) preventing eosinophil and/or basophil and/or dendritic cell and/or neutrophil and/or monocyte recruitment (see e.g. Miyahara N et al, Immunol. 2005 Apr. 15; 174(8); 4979-84); (x) abnormal bone remodeling, loss or gain, including osteopenia, osteoporosis, Paget's disease, cancer and other diseases (see e.g. Anderson G I, et al., Biomed Mater Res. 2001; 58(4):406-140; (xi) ocular inflammation and allergic conjunctivitis, vernal keratoconjunctivitis, and papillary conjunctivitis (see e.g. Lambiase et al, Arch. Opthalmol., v121, 615-620 (2003)); (xii) CMS disorders, including, but are not limited to, multiple sclerosis, Parkinson's disease, Alzheimer's disease, stroke, cerebral ischemia, retinal ischemia, post-surgical cognitive dysfunction, migraine (see e.g. de Souza Carvalho D, et al., Headache. 2002 November-December; 42(10): 1044-7; Sheftell F, et al., Headache. 2000 February; 40(2):158-63); (xiii) peripheral neuropathy/neuropathic pain, spinal cord injury (see e.g. Akpek E A, et al., Spine. 1999 Jan. 15; 24(2): 128-32), cerebral edema and head injury; (xiv) cancer, including, but is not limited to, pancreatic cancer and other solid or hematological tumors, (see e.g. Poff and Balazy, Curr. Drug Targets Inflamm. Allergy, v3, 19-33 (2004) and Steele et al, Cancer Epidemiology & Prevention, v8, 467-483 (1999); (xv) endotoxic shock and septic shock (see e.g. Leite M S, et al., Shock, 2005 February; 23(2):173-8); (xvi) rheumatoid arthritis and osteoarthritis (see e.g. Alien R, et al., Ann Rheum Dis. 2004 February; 63(2):170-6); (xvii) preventing increased GI diseases, including, by way of example only, chronic gastritis, eosinophilic gastroenteritis, and gastric motor dysfunction, (see e.g. Gyomber et al, J Gastroenterol Hepatol., v11, 922-927 (1996); Quack I et al., BMC Gastroenterol v18, 24 (2005); Cuzzocrea S, et al., Lab Invest. 2005 June; 85(6):808-22); (xviii) kidney diseases, including, by way of example only, glomerulonephritis, cyclosporine nephrotoxicity renal ischemia reperfusion, (see e.g. Guasch et al., Kidney Int., v56, 261-267; Butterly et al, Kidney Int., v 57, 2586-2593 (2000): Guasch A et al. Kidney Int. 1999; 56:261-7; Butterly D W et al Kidney Int. 2000; 57:2586-93); (xix) preventing or treating acute or chronic renal insufficiency (see e.g. Maccarrone M, et al., J Am Soc Nephrol. 1999; 10:1991-6); (xx) diminish the inflammatory aspects of acute infections within one or more solid organs or tissues such as the kidney with acute pyelonephritis (see e.g. Tardif M, et al., Antimicrob Agents Chemother. 1994 July; 38(7): 1555-60); (xxi) preventing or treating acute or chronic disorders involving recruitment or activation of eosinophils (see e.g. Quack I, et al BMC Gastroenterol., 2005; 5:24; (xxii) preventing or treating acute or chronic erosive disease or motor dysfunction of the gastrointestinal tract caused by non-steroidal anti-inflammatory drugs (including selective or non-selective cyclooxygenase-1 or -2 inhibitors) (see e.g. Marusova I B, et al., Eksp Klin Farmakol, 2002; 65:16-8 and Gyomber E, et al., J. Gastroenterol. Hepatol, 1996, 11, 922-7) and Martin St et al., Eur J Gastroenterol. Hepatol., 2005, 17:983-6; (xxiii) treating type II diabetes (see e.g. Valdivielso J M, et al., J Nephrol. 2003 January-February; 16(1):85-94; Parlapiano C, et al., Diabetes Res Clin Pract. 1999 October; 46(1):43-5; (xxiv) treatment of metabolic syndromes, including, by way of example only. Familial Mediterranean Fever (see e.g. Bentancur A G, et al., Clin Exp Rheumatol. 2004 July-August; 22(4 Suppl 34):S56-8; (xxv) treat hepatorenal syndrome (see e.g. Capella G L., Prostaglandins Leukot Essent Fatty Acids. 2003 April; 68(4):263-53; and (xxvi) pulmonary hypertension.

Several inhibitors of FLAP have been described, the disclosures of which are incorporated by reference in their entirety as examples of agents that can be used in the combination compositions and methods described herein: Gillard et al, Can. J. Physiol. Pharmacol., 67, 456-464, 1989; Evans et al, Molecular Pharmacol, 40, 22-27, 1991; Brideau et al, Can. J. Physiol. Pharmacol, 1992 June; 70(6); 799-807; Musser et al, J. Med. Chem., 35, 2501-2524, 1992; Steinhilber, Curr. Med. Chem. 6(1):71-85, 1999; Riendeau, Bioorg Med Chem Lett., 15(14):3352-5, 2005; Flamand, et al., Mol. Pharmacol. 62(2):250-6, 2002; Folco, et al., Am. J. Respir. Crit. Care Med 161(2 Pt 2):S112-6, 2000; Hakonarson, JAMA, 293(18):2245-56, 2005.

Non-limiting examples of agents which bind FLAP and inhibit the synthesis of leukotrienes are described in the following publications, the disclosures of which are incorporated by reference in their entirety: Ford-Hutchinson, et al., Ann. Rev. Biochem., 63:383-417(1994); Rouzer, et al., J. Biol. Chem., 265:1436-42 (1990): and Gorenne, et al., J. Pharmacol Exp. Ther., 268:868-72 (1994). Further non-limiting examples of agents which bind FLAP are described in the following patents and patent applications, the disclosures of which are incorporated by reference in their entirety as examples of agents that can be used in the combination compositions and methods described herein: U.S. Pat. Nos. 6,756,399 and 6,436,924, and U.S. Patent Application Publication Nos.: 20020022650 and 20010025040. In addition, the following FLAP inhibitors are examples of agents that can be used in the combination compositions and methods described herein; Abbott-72694; Abbott-81834; Abbott-93178; indole and quinoline compounds such as MK-591, MK-886, BAY X 1005, BAY Y 1015., ETH603, ETH615, ETH647, WAY-123520, and WY 50295. The FLAP inhibitor may also be selected from compounds described in U.S. Pat. Nos. 4,929,626; 4,970,215; 5,081,138; 5,095,031; 5,204,344; 5,126,354; 5,221,678; 5,229,516; 5,272,145; 5,283,252; 5,288,743; 5,292,769; 5,304,563; 5,399,699; 5,459,150; 5,512,581; 5,597,833; 5,668,146; 5,668,150; 5,691,351; 5,714,488; 5,783,586; 5,795,900; and 5,843,968.

Further non-limiting examples of FLAP inhibitors are described in the following patent publications, the disclosures of which are incorporated by reference in their entirety as examples of agents that can be used in the combination compositions and methods described herein: U.S. patent publication no. 2007/0123522, U.S. patent publication no. 2007/0105866, U.S. patent publication no. 2007/0219206, U.S. patent publication no. 2007/0225285, U.S. patent publication no. 2007/0244128, International patent publication no. WO 07/056,021, International patent publication no. WO07/056,220, International patent publication no. WO07/056,338, International patent publication no. WO07/047207.

The FLAP inhibitor may also be selected from (the following compounds (a)-(h) are referred to collectively as “FLAP compounds (a)-(j)”):

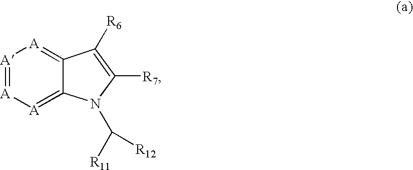

wherein, each A is independently selected from N or CR₅, and each A′ is C—Z—Y, N or CR₅, provided that one A′ is C—Z—Y and the other A′ is N or CR₅ and provided that the number of N groups from a plus the number of N groups from A′ is 1 or 2;
Z is selected from a bond, —CR₁═CR₁—, —C≡C—, —C(R₂)ₙ—, —C(R₁)₂O—, —OC(R₁)₂—, —C(R₁)₂S(O)ₘ—, —S(O)ₘC(R₁)₂—, —C(R₁)₂NH—, —NHC(R₁)₂—, —C(R₂)₂C(R₁)₂O—, —C(R₁)₂OC(R₁)₂—, —OC(R₁)₂C(R₂)₂—, —C(O)NH—, —NHC(═O)—, wherein each R₁ is independently H, CF₃, or an optionally substituted lower alkyl; and each R₂ is independently H, OH, OMe, CF₃, or an optionally substituted lower alkyl; m is 0, 1 or 2; n is 0, 1, 2, or 3;
Y is a -L₁-(substituted or unsubstituted heterocycloalkyl), -L₁-(substituted or unsubstituted heteroaryl), -L₁-(substituted or unsubstituted aryl) or -L₁-C(═NR₃)N(R₄)₂, -L₁-NR₄C(═NR₃)N(R₄)₂, -L₁-NR₄C(═CHR₃)N(R₄)₂;
where R₃ is independently selected from H, S(═O)₂R₄, —S(═O)₂NH₂—C(O)R₄, —CN, —NO₂, heteroaryl, or heteroalkyl;
each R₄ is independently selected from H, substituted or unsubstituted lower alkyl, substituted or unsubstituted lower cycloalkyl; or two R₄ groups can together form a 5-, 6-, 7-, or 8-membered heterocyclic ring; or R₃ and R₄ groups can together form a 5-, 6-, 7-, or 8-membered heterocyclic ring;
where L₁ is a bond, a substituted or unsubstituted alkyl, a substituted or unsubstituted alkenyl a substituted or unsubstituted alkynyl, a substituted or unsubstituted heteroalkyl;
R₆ is H, -L₂-(substituted or unsubstituted alkyl), -L₂-(substituted or unsubstituted cycloalkyl), -L₂-(substituted or unsubstituted alkenyl), -L₂-(substituted or unsubstituted cycloalkenyl), -L₂-(substituted or unsubstituted heterocycloalkyl), -L₂-(substituted or unsubstituted heteroaryl), or -L₂-(substituted or unsubstituted aryl), where L₂ is a bond, —O—, —S—, —S(═O)—, —S(═O)₂—, —C(═O)—, -(substituted or unsubstituted C₁-C₆ alkyl), or -(substituted or unsubstituted C₂-C₆ alkenyl);
R₇ is L₃-X-L₃-G, wherein,
X is a bond, —O—, —C(═O)—, —S—, —S(═O)—, —S(═O)₂—, —NH—, —NR₈, —NHC(═O)—, —C(═O)NH—, —NR₈C(═O)—, —C(═O)NR₈, —S(═O)₂NH—, —NHS(═O)₂—, —S(═O)₂NR₈, —NR₈S(═O)₂—, —OC(═O)NH—, —NHC(═O)O—, —OC(═O)NR₈—, —NR₈C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—;
L₃ is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycloalkyl;
L₄ is a bond, optionally substituted or unsubstituted alkyl, optionally substituted or unsubstituted cycloalkyl, optionally substituted or unsubstituted alkenyl, optionally substituted or unsubstituted alkynyl;
G is H, —CO₃H, tetrazolyl, —NHS(═O)₂R₈, —S(═O)₂N(R₉)₂, —OH, —OR₈, —C(═O)CF₃, —C(O)NHS(═O)₂R₈, —S(═)₂NHC(O)R₈, —CN, —N(R₉)₂, —C(═NR₁₀)N(R₈)₂, —NR₉C(═NR₁₀)N(R₉)₂, —NR₉C(═CHR₁₀)N(R₉)₂, —CO₃R₈, —C(═O)R₈, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(═O)O—, —NHC(═O)—, —C(═O)NH—, —C(═O)O—, or —OC(═O)—;
or G is W-G₁, where W is a substituted or unsubstituted aryl, substituted or unsubstituted heterocycloalkyl or substituted or unsubstituted heteroaryl and G₁ is H, —CO₂H, tetrazolyl, —NHS(═O)₂R₈, —S(═O)₂N(R₉), —OH, —OR₈, —C(═O)CF₈, —C(O)NHS(═O)₂R₈, —S(═O)₂NHC(O)R₈, —CN, N(R₉)₂, —C(═NR₁₀)N(R₉)₂—NR₉C(═NR₁₀)N(R₉)₂, —NR₉C(═CHR₁₀)N(R₉)₂, —CO₂R₈, —C(═O)NH₂, —C(═O)NHR₈, or —C(═O)N(R₈)₂;
each R₈ is independently selected from substituted or unsubstituted lower alkyl, substituted or unsubstituted lower cycloalkyl;
each R₉ is independently selected from H, substituted or unsubstituted lower alkyl, substituted or unsubstituted lower cycloalkyl; or two R₉ groups can together form a 5-, 6-, 7-, or 8-membered heterocyclic ring; or R₈ and R₉ can together form a 5-, 6-, 7-, or 8-membered heterocyclic ring;
each R₁₀ is independently selected from H, —S(═O)₂R₈, —S(═O)₂NH₂, —C(═O)R₈, —CN, —NO₂, heteroaryl or heteroalkyl;
R₅ is H, halogen, —N₃, —CN, —ONO₂, -L₆-(substituted or unsubstituted C₁-C₃ alkyl), -L₆-(substituted or unsubstituted C₂-C₄ alkenyl), -L₆-(substituted or unsubstituted heteroaryl), or -L₆-(substituted or unsubstituted aryl), wherein L₆ is a bond, —O—, —S—, —S(═O)—, —S(═O)—, —NH—, —C(═O)—, —NHC(═O)O—, —OC(═O)NH—, —NHC(═O)—, or —C(═O)NH—;
R₁₁ is L₇-G, L₇-(substituted or unsubstituted cycloalkyl)-G, L₇-(substituted or unsubstituted cycloalkenyl)-G, L₇-(substituted or unsubstituted heteroaryl)-G, or L₇-(substituted or unsubstituted aryl)-G, L₇-(substituted or unsubstituted heterocycloalkyl)-G, where L₇ is a bond, —C(═O)—, —C(═O)NH—, (substituted or unsubstituted C₁-C₆ alkyl), or (substituted or unsubstituted C₂-C₆ alkenyl);
R₁₂ is H, or L₈-L₉-R₁₃, wherein L₈ is a bond, (substituted or unsubstituted C₁-C₆ alkyl), or (substituted or unsubstituted C₂-C₄ alkenyl); L₉ is a bond, —O—, —S—, —S(═O)—, —S(═O)₂—, —NH—, —C(═O)O—, —NHC(═O)O—, —OC(═O)NH—, —NHC(═O)—, —C(═O)NH—, —C(═O)O—, or —OC(═O)—; R₁₃, is H, (substituted or unsubstituted C₁-C₆ alkyl), (substituted or unsubstituted aryl), (substituted or unsubstituted heteroaryl), or (substituted or unsubstituted heterocycloalkyl);
or R₇ and R₁₂ can together form a 4 to 8-membered heterocyclic ring;
or solvate, or pharmaceutically acceptable salt, or a pharmaceutically acceptable prodrug thereof;

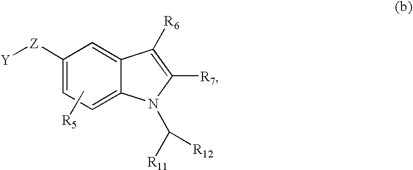

wherein, Z is selected from —N(R₁)—, —S(═O)ₘ—, —CR₁═CR₁—, —C≡C—, —C(R₁)₂[C(R₂)₂]ₙ—, —[C(R₂)₂]ₙC(R₁)₂O—, —OC(R₁)₂[C(R₂)₂]ₙ—, —[C(R₂)₂]ₙC(R₁)₂S(O)ₘ—, —S(O)ₘC(R₁)₂[C(R₂)₂]ₙ—, —[C(R₂)₂]ₙC(R₁)₂NR₁—, —NR₁C(R₁)₂[C(R₂)₂]ₙ—, —[C(R₂)₂]ₙO[C(R₁)₂]ₙ—, —[C(R₁)₂]ₙO[C(R₂)₂]ₙ—, —C(═O)NR₂—, —NR₂C(═O)—, —NR₂C(═O)O—, —OC(═O)NR₂—, —S(═O)₂NR₂—, —CR₁═N—N—, —NR₂C(═O)NR₂—, —OC(═O)O—, S(═O)₂NR₂, or —NR₂S(═O)₂—, wherein each R₁ is independently H, CF₃, or an optionally substituted lower alkyl, or two R₁, on the same carbon may join to form an oxo (═O); each R₂ is independently selected from H, OH, OMe, CF₃, or an optionally substituted lower alkyl, or two R₂ on the same carbon may join to form an oxo (═O); m is 0, 1 or 2; each n is independently 0, 1, 2, or 3;
Y is H, —CO₂H, tetrazolyl, —NHS(═O)₂R_{3b}, —S(═O)₂N(R₄)₂, —OH, —OR_{3b}, —C(═O)(C₁-C₅ fluoroalkyl), —C(═O)NHS(═O)₂R_{3b}, —S(═O)₂NHC(O)R₄, —CN, —N(R₄)₂, —N(R₄)C(═O)R₄, —C(═NR₃)N(R₄)₂, —NR₄C(═NR₃)H(R₄)₂, —NR₄C(═CHR₃)N(R₄)₂, —C(═O)NR₄C(═NR₃)N(R₄)₂, —C(O)NR₄C(═CHR₃)N(R₄)₂, —CO₂R_{3b}, —C(═O)R₄, —C(═O)N(R₄)₂, —SR_{3b}, —S(═O)R_{3b}, —S(═O)₂R_{3b}, -L₁-(substituted or unsubstituted alkyl), -L₁-(substituted or unsubstituted alkenyl), -L₁-(substituted or unsubstituted alkynyl), -L₁-(substituted or unsubstituted cycloalkyl), -L₁-(substituted or unsubstituted heterocycloalkyl), -L₁-(substituted or unsubstituted heteroaryl), -L₁-(substituted or unsubstituted aryl), -L₁-C(═NR₄)N(R₄)₂, -L₁-NR₄C(═NR₄)N(R₄)₂, or -L₁-NR₄C(═CR₃)N(R₄)₂;
where L₁ is a bond, a substituted or unsubstituted alkyl, substituted or unsubstituted alkenyl, substituted or unsubstituted alkynyl, a substituted or unsubstituted heterocycloalkyl, a substituted or unsubstituted heteroaryl, a substituted or unsubstituted cycloalkyl, a substituted or unsubstituted heteroalkyl, substituted or unsubstituted heteroalkenyl a substituted, or unsubstituted heteroalkynyl, or substituted or unsubstituted aryl;
where each substituent is -LₛRₛ, wherein each Lₛ is independently selected from a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, C₁-C₆ alkyl, C₂-C₆ alkenyl, —C₁-C₆ fluoroalkyl, heteroaryl, aryl, or heterocycloalkyl; and each Rₛ is independently selected from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl;
each R₃ is independently selected from H, —S(═O)₂R₈, —S(═O)₂NH₂—C(O)R₈, —CN, —NO₂, heteroaryl, or heteroalkyl; each R_{3b} is independently selected from substituted or unsubstituted lower alkyl, substituted or unsubstituted lower cycloalkyl, phenyl or benzyl; each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycloalkyl), L₂-(substituted or unsubstituted heteroaryl), or L₂-substituted or unsubstituted aryl), where L₂ is a bond, —O—, —S—, —S(═O)—, —S(═O)₂, —C(═O)—, —CH(OH)—, -(substituted or unsubstituted C₁-C₆ alkyl), or -(substituted or unsubstituted C₂-C₆ alkenyl);
R₇ is selected from
(i) L₃-X-L₄-G₁, wherein, L₃ is a substituted or unsubstituted alkenyl substituted or unsubstituted alkynyl, substituted or unsubstituted aryl, substituted or unsubstituted heteroaryl, substituted or unsubstituted heterocycloalkyl; X is a bond, —O—, —C(═O)—, —CR₉(OR₉)—, —S—, —S(═O)—, —S(═O)₂—, —NR₉—, —NR₉C(═O)—, —C(O)NR₉—, —S(═O)₂NR₉—, —NR₉S(═O)₂—, —OC(═O)NR₉—, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₄ is a bond, substituted or unsubstituted alkyl, substituted or unsubstituted cycloalkyl, substituted or unsubstituted alkenyl, substituted or unsubstituted alkynyl; G₁ is H tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, —CN, —N(R₉)₂, —N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted of unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —O(═O)CNH—, —NHC(═O)—, —C(═O)NH—, —C(═O)O—, or —OC(═O)—; or G₁ is W-G₅, where W is a substituted or unsubstituted aryl, substituted or unsubstituted heterocycloalkyl or substituted or unsubstituted heteroaryl; G₅ is H, tetrazolyl —NHS (═O)₂R₈, S(═O)₂N(R₈)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl, phenyl or benzyl;
each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, —S(═O)₂R₈, —S(═O)₂NH₂—C(═O)R₈, —CN, —NO₂, heteroaryl, or heteroalkyl;
(ii) L₃-X-L₃-G₂, wherein, L₃ is a bond, substituted or unsubstituted alkyl, substituted or unsubstituted cycloalkyl, substituted or unsubstituted alkenyl, substituted or unsubstituted alkynyl, substituted or unsubstituted aryl substituted or unsubstituted heteroaryl, substituted or unsubstituted heterocycloalkyl; X is —NR₉C(═O))—, —C(═O)NR₉—, —S(═O)₂NR₉—, —NR₉S(═O)₂, —OC(═O)NR₉, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₄ is a bond, substituted or unsubstituted alkyl, substituted or unsubstituted cycloalkyl, substituted or unsubstituted alkenyl, substituted or unsubstituted alkynyl; G₂ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, —CN, —N(R₉)₂, —N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O, —OC(═O)NH—, —NHC(═O)—, —C(═O)NH—, —C(O)O—, or —OC(O)—; or G₂ is W-G₅, where W is a substituted or unsubstituted aryl, substituted or unsubstituted heterocycloalkyl, or substituted or unsubstituted heteroaryl; and G₅ is H, tetrazolyl, —NHS(═O)₂R₈, —S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl phenyl or benzyl;
each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, —S(═O)₂R₈, —S(═O)₂NH₂—C(═O)R₈, —CN, —NO₂, heteroaryl, or heteroalkyl;
(iii) L₃-X-L₃-G₃, wherein, X is a bond, —O—, —C(═O)—, —CR₉(OR₉)—, —S—, —S(═O)—, —S(═O)₂—, —NR₉—, —NR₉C(═O)—, —C(═O)NR₉, —S(═O)₂NR₉—, —NR₉S(═O)₂, —OC(═O)NR₉, —, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₃ is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycloalkyl; L₄ is a (substituted or unsubstituted alkenyl) or (substituted or unsubstituted alkynyl); G₃ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, —CN, —N(R₉)₂, —N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—; or G₃ is W-G₅, where W is a substituted or unsubstituted aryl, substituted or unsubstituted heterocycloalkyl or substituted or unsubstituted heteroaryl; and G₅ is H, tetrazolyl, —NHS(═O)₂R₈, —S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl, phenyl or benzyl;
each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic, ring and each R₁₀ is independently selected from H, —S(═O)₂R₈, —S(═O)₂NH₂, —C(═O)R₈, —CN, —NO₂, heteroaryl, or heteroalkyl;
or (iv) L₃-X-L₄₋G₄, wherein, L₃ is a bond, substituted or unsubstituted alkyl, substituted or unsubstituted cycloalkyl, substituted or unsubstituted alkenyl substituted or unsubstituted alkynyl, substituted or unsubstituted aryl, substituted or unsubstituted heteroaryl, substituted or unsubstituted heterocycloalkyl; X is a bond, —O—, —C(═O)—, —CR₉(OR₉)—, —S—, —S(═O)—, —S(═O)₂—, —NR₉—, —NR₉C(═O)—, —C(═O)NR₉—, —S(═O)₂NR₉—, —NR₉S(═O)₂—, —OC(═O)NR₉—, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₄ is a bond, substituted or unsubstituted alkyl, substituted or unsubstituted cycloalkyl, substituted or unsubstituted alkenyl, substituted or unsubstituted alkynyl; G₄ is —C(═NR₁₀)N(R₉)₂, —NR₉C(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-substituted or unsubstituted aryl), wherein L₅ is —NHC(═O)O—, —OC(═O)NH—, —C(═O)O—, or —OC(═O)—; or G₄ is -L₅-(substituted or unsubstituted alkenyl), -L₅-(substituted or unsubstituted heteroaryl), or -L₅-(substituted or unsubstituted aryl), wherein L₅ is —NHC(═O)O—, —OC(═O)NH—, —NHC(═O)—, —C(═O)NH—, —C(═O)O—, or —OC(═O)—; or G₄ is W-G₅, where W is a substituted or unsubstituted aryl, substituted or unsubstituted heterocycloalkyl, or substituted or unsubstituted heteroaryl and G₅ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(═O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —(═O)R₉, —C(═O)N(R₉)₂, —SR₈, —S(═O)R₈, or —S(═O)₂R₈; each R₈ is independently selected from substituted or unsubstituted lower alkyl substituted or unsubstituted lower cycloalkyl, phenyl or benzyl;
each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, —S(═O)₂R₈, —S(═O)₂NH₂—C(═O)R₈, —CN, —NO₂, heteroaryl, or heteroalkyl;
R₈ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, S(═O)>, —NH—, —C(═O)—, —NHC(═O)O—, —OC(═O)NH—, —NHC(═O)—, —NHC(═O)NH, or —C(═O)NH—;
R₁₁ is L₇-L₁₀-G₆; wherein L₇ is a bond, —O—, —S—, —S(═O)—, —S(═O)₂—, —NH—, —C(═O)—, —C(═O)NH—, —NHC(═O)—, (substituted or unsubstituted C₁-C₆ alkyl), or (substituted or unsubstituted C₂-C₆ alkenyl): L₁₀ is a bond, (substituted or unsubstituted alkyl), (substituted or unsubstituted cycloalkyl), (substituted or unsubstituted cycloalkenyl), (substituted or unsubstituted heteroaryl), (substituted or unsubstituted and), or (substituted or unsubstituted heterocycloalkyl); and G₆ is H, —CN, —SCN, —N₃, —NO₂, halogen, —OR₉, —C(═O)CF₃, —C(═O)R₉, —SR₈, —S(═O)R₈, —S(═O)₂R₈, —N(R₉)₂, tetrazolyl, —NHS(═O)₂R₈, —S(═O)₂N(R₉)₂, —C(═O)NHS(═O)₂R₈, —-L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(═O)—, —NHC(═O)NH—, —OC(═O)O—, —OC(═O)NH—, —NHC(═O)—, —C(═O)NH—, —C(═O)O—, or —OC(═O)—; or G₆ is W-G₇, wherein W is (substituted or unsubstituted cycloalkyl), (substituted or unsubstituted cycloalkenyl), (substituted or unsubstituted aryl), (substituted or unsubstituted heterocycloalkyl) or a (substituted or unsubstituted heteroaryl); and G₇ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —C(═O)N(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is —NH—, —NHC(═O)O—, —NHC(═O)NH—, —OC(═O)O—, —OC(═O)NH—, —NHC(═O)—, —C(═O)NH—, —C(═O)O—, or —OC(═O)—; and
R₁₂ is L₈-L₉-R₁₃, wherein L₈ is a bond, (substituted or unsubstituted C₁-C₆, alkyl), or (substituted or unsubstituted C₂-C₄ alkenyl); L₉ is a bond, —O—, —S—, —S(═O)—, —S(═O)₂—, —NH—, —C(═O)—, —NHC(═O)O—, —OC(═O)NH—, —NHC(═O)NH—, —OC(═O)O—, —NHC(═O)—, —C(═O)NH—, —C(═O)O—, or —OC(═O)—; R₁₃ is H, (substituted or unsubstituted C₁-C₆ alkyl), (substituted or unsubstituted C₃-C₆ cycloalkyl), (substituted or unsubstituted aryl), (substituted or unsubstituted heteroaryl), or (substituted or unsubstituted heterocycloalkyl);
or R₇ and R₁₂ can together form a 4 to 8-membered heterocyclic ring;

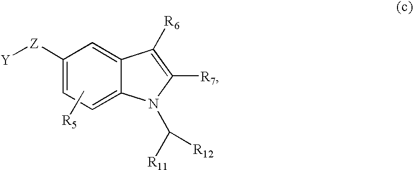

wherein, Z is selected from —N(R₁)—, —S(═O)ₘ—, —CR₁═CR₁—, —C≡C—, —C(R₁)₂[C(R₂)₂]ₙ—, —[C(R₂)₂]ₙC(R₁)₂O—, —OC(R₁)₂[C(R₂)₂]ₙ—, —[C(R₂)₂]ₙC(R₁)₂S(O)ₘ—, —S(O)ₘC(R₁)₂[C(R₂)₂]ₙ—, —[C(R₂)₂]ₙC(R₁)₂NR₁—, —NR₁C(R₁)₂[C(R₂)₂]ₙ—, —[C(R₂)₂]ₙO[C(R₁)₂]ₙ—, —[C(R₁)₂]ₙO[C(R₂)₂]ₙ—, —C(═O)NR₂—, —NR₂C(═O)—, —NR₂C(═O)O—, —OC(═O)NR₂—, —S(═O)₂NR₂—, —CR₁═N—N—, —NR₂C(═O)NR₂—, —OC(═O)O—, S(═O)₂NR₂, or —NR₂S(═O)₂—, wherein each R₁ is independently H, CF₂, or an optionally substituted lower alkyl; or two R₁, groups on the same carbon may join to form an oxo (═O); each R₂ is independently H, OH, OMe, CF₃, or an optionally substituted lower alkyl; or two R₂ groups on the same carbon may join to form an oxo (═O); m is 0, 1 or 2; each n is independently 0, 1, 2, or 3;
Y is H, —CO₂H, tetrazolyl, —NHS(═O)₂R_{3b}, —S(═O)₂N(R₄)₂, —OH, —OR_{3b}, —C(═O)(C₁-C₅ fluoroalkyl), —C(═O)NHS(═O)₂R_{3b}, —S(═O)₂NHC(O)R₄, —CN, —N(R₄)₂, —N(R₄)C(═O)R₄, —C(═NR₃)N(R₄)₂, —NR₄C(═NR₃)H(R₄)₂, —NR₄C(═CHR₃)N(R₄)₂, —C(═O)NR₄C(═NR₃)N(R₄)₂, —C(O)NR₄C(═CHR₃)N(R₄)₂, —CO₂R_{3b}, —C(═O)R₄, —C(═O)N(R₄)₂, —SR_{3b}, —S(═O)R_{3b}, —S(═O)₂R_{3b}, -L₁-(substituted or unsubstituted alkyl), -L₁-(substituted or unsubstituted alkenyl), -L₁-(substituted or unsubstituted alkynyl), -L₁-(substituted or unsubstituted cycloalkyl), -L₁-(substituted or unsubstituted heterocycloalkyl), -L₁-(substituted or unsubstituted heteroaryl), -L₁-(substituted or unsubstituted aryl), -L₁-C(═NR₄)N(R₄)₂, -L₁-NR₄C(═NR₄)N(R₄)₂, or -L₁-NR₄C(═CR₃)N(R₄)₂;
where L₁ is a bond, a substituted or unsubstituted alkyl, substituted or unsubstituted alkenyl, substituted or unsubstituted alkynyl, a substituted or unsubstituted heterocycloalkyl a substituted or unsubstituted heteroaryl, a substituted or unsubstituted cycloalkyl, a substituted or unsubstituted heteroalkyl substituted or unsubstituted heteroalkenyl a substituted or unsubstituted heteroalkynyl, or substituted or unsubstituted aryl;
where each substituent is -LₛRₛ, wherein each Lₛ is independently selected from a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, C₁-C₆ alkyl, C₂-C₆ alkenyl, —C₁-C₆ fluoroalkyl, heteroaryl aryl, or heterocycloalkyl; and each Rₛ is independently selected from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl;
each R₃ is independently selected from H, —S(═O)₂R₈, —S(═O)₂NH₂, —C(O)R₈, —CN, —NO₂, heteroaryl, or heteroalkyl; each R_{3b} is independently selected from substituted or unsubstituted lower alkyl, substituted or unsubstituted lower cycloalkyl phenyl or benzyl; each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycloalkyl), L₂-(substituted or substituted heteroaryl), or L₂-(substituted or unsubstituted aryl), where L₂ is a bond, —O—, —S—, —S(═O)—, —S(═O)₂—, —C(═O)—, —CH(OH)—, -(substituted or unsubstituted C₁-C₆alkyl), or -(substituted or unsubstituted C₁-C₆ alkenyl);
R₇ is H or substituted or unsubstituted alkyl;
R₆ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, —S(═O)₂—, —NH—, —C(═O)—, —NHC(═O)O—, —OC(═O)NH, —NHC(═O), —NHC(═O)NH—, or —C(═O)NH;
R₁₁ is L₇₋L₁₀-G₆; wherein L₇ is a bond, —O—, —S—, —S(═O)—, —S(═O)₂, —NH—, —C(═O), —C(═O)NH—, —NHC(═O)—, (substituted or unsubstituted C₁-C₆ alkyl), or (substituted or unsubstituted C₂-C₆ alkenyl); L₁₀ is a bond, (substituted or unsubstituted alkyl), (substituted or unsubstituted cycloalkyl), (substituted or unsubstituted cycloalkenyl), (substituted or unsubstituted heteroaryl), (substituted or unsubstituted aryl), or (substituted or unsubstituted heterocycle), and G₆ is H, —CN, —SCN, —N₃, —NO₂, halogen, —OR₉, —C(═O)CF₃, —C(═O)R₉, —SR₈, —S(═O)R₈, —S(═O)₂R₈, —N(R₉)₂, tetrazolyl, —NHS(═O)₂R₈, —S(═O)₂N(R₉)₂, —C(═O)NHS(═O)₂R₈, —S(═O)₂NHC(═O)R₉, —C(═NR₁₀)N(R₉)₂, —NR₉(═NR₁₀)N(R₉)₂, —NR₉C(═CR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(═O)O—, —NHC(═O)NH—, —OC(═O)O—, —OC(═O)NH—, —NHC(═O)—, —C(═O)NH, —C(═O)O—, or —OC(═O)—: or G₆ is W-G₇, wherein W is (substituted or unsubstituted cycloalkyl), (substituted or unsubstituted cycloalkenyl), (substituted or unsubstituted aryl), (substituted or unsubstituted heterocycloalkyl) or a (substituted or unsubstituted heteroaryl); and G₇ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, or —S(═O)₂R₈, -L₅-(substituted or unsubstituted alkyl), -L₅-(substituted, or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is —NH—, —NHC(═O)O—, —NHC(═O)NH—, —OC(═O)O—, —OC(═O)NH—, —NHC(═O)—, —C(═O)NH, —C(═O)O, or —OC(═O)—;
R₁₂ is L₃-X-L₄-G₁, wherein, L₃ is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl substituted or unsubstituted heterocycle; X is a bond, —O—, —C(═O)—, —CR₉(OR₉)—, —S—, —S(═O)—, —S(═O)₂—, —NR₉—, —NR₉C(═O)—, —C(═O)NR₉—, —S(═O)₂NR₉—, —NR₉S(═O)₂—, —OC(═O)NR₉—, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀—, or —C(═NR₁₀)O—; L₄ is bond, substituted or unsubstituted alkyl, substituted or unsubstituted cycloalkyl substituted or unsubstituted alkenyl substituted or unsubstituted alkynyl; G₁ is tetrazolyl, —NHS(═O)₂R₈, S(═O)₂N(R₉)₂, —OR₉, —C(═O)CF₃, —C(═O)NHS(═O)₂R₈, —S(═O)₂NHC(═O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—; or G₁ is W-G₅, where W is a substituted or unsubstituted aryl, substituted or unsubstituted heterocycloalkyl, or substituted or unsubstituted heteroaryl; and G₅ is H, tetrazolyl, —NHS(═O)₂R₈, —S(═O)₂N(R₉)₂, —OH, —OR₈, —C(═O)CF₃, —C(═O)NHS(═O)₂R₈, —S(═O)₂NHC(═O)R₉, —CN, N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(O)NR₉C(═CHR₁₀)N(R₉)₂, —CO₂R₉, —C(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl, phenyl or benzyl; each R₉ is independently selected front H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, —S(═O)₂R₈, —S(═O)₂NH₂—C(═O)R₈, —CN, —NO₂, heteroaryl, or heteroalkyl;

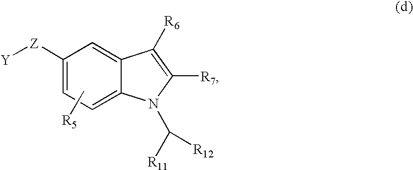

wherein, Z is selected from —N(R₁)—, —S(═O)ₘ—, —CR₁═CR₁—, —C≡C—, —C(R₁)₂[C(R₂)₂]ₙ—, —[C(R₂)₂]ₙC(R₁)₂O—, —OC(R₁)₂[C(R₂)₂]ₙ—, —[C(R₂)₂]ₙC(R₁)₂S(O)ₘ—, —S(O)ₘC(R₁)₂[C(R₂)₂]ₙ—, —[C(R₂)₂]ₙC(R₁)₂NR₁—, —NR₁C(R₁)₂[C(R₂)₂]ₙ—, —[C(R₂)₂]ₙO[C(R₁)₂]ₙ—, —[C(R₁)₂]ₙO[C(R₂)₂]ₙ—, —C(O)NR₂—, —NR₂C(═O)—, —NR₂C(═O)O—, —OC(═O)NR₂—, —S(═O)₂NR₂—, —CR₁═N—N—, —NR₂C(═O)NR₂—, —OC(═O)O—, S(═O)₂NR₂, or —NR₂S(═O)₂—, wherein each R₁ is independently H, CF₃, or an optionally substituted lower alkyl; or two R₁ groups on the same carbon may join to form an oxo (═O) each R₂ is independently H, —OH, —OMe, —CF₃, or an optionally substituted lower alkyl; or two R₂ groups on the same carbon may join to form an oxo (═O); m is 0, 1 or 2; each n is independently 0, 1, 2, or 3;
Y is H, —CO₂H, tetrazolyl, —NHS(═O)₂R_{3b}, —S(═O)₂N(R₄)₂, —OH, —OR_{3b}, —C(═O)(C₁-C₅ fluoroalkyl), —C(═O)NHS(═O)₂R_{3b}, —S(═O)₂NHC(O)R₄, —CN, —N(R₄)₂, —N(R₄)C(═O)R₄, —C(═NR₃)N(R₄)₂, —NR₄C(═NR₃)H(R₄)₂, —NR₄C(═CHR₃)N(R₄)₂, —C(═O)NR₄C(═NR₃)N(R₄)₂, —C(O)NR₄C(═CHR₃)N(R₄)₂, —CO₂R_{3b}, —C(═O)R₄, —C(═O)N(R₄)₂, —SR_{3b}, —S(═O)R_{3b}, —S(═O)₂R_{3b}, -L₁-(substituted or unsubstituted alkyl), -L₁-(substituted or unsubstituted alkenyl), -L₁-(substituted or unsubstituted alkynyl), -L₁-(substituted or unsubstituted cycloalkyl), -L₁-(substituted or unsubstituted heterocycloalkyl), -L₁-(substituted or unsubstituted heteroaryl), -L₁-(substituted or unsubstituted aryl), -L₁-C(═NR₄)N(R₄)₂, -L₁-NR₄C(═NR₄)N(R₄)₂, or -L₁-NR₄C(═CR₃)N(R₄)₂;
where L₁ is a bond, a substituted or unsubstituted alkyl, substituted or unsubstituted alkenyl, substituted or unsubstituted alkynyl, a substituted or unsubstituted heterocycloalkyl, a substituted or unsubstituted heteroaryl, a substituted or unsubstituted cycloalkyl, a substituted or unsubstituted heteroalkyl, substituted or unsubstituted heteroalkenyl a substituted, or unsubstituted heteroalkynyl, or substituted or unsubstituted aryl;
where each substituent is -LₛRₛ, wherein each -Lₛ- is independently selected from a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, C₁-C₆ alkyl, C₂-C₆ alkenyl, —C₁-C₆ fluoroalkyl, heteroaryl aryl, or heterocycloalkyl; and each Rₛ is independently selected from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl;
each R₃ is independently selected from H, —S(═O)₂R₈, —S(═O)₂NH₂, —C(═O)R₈, —CN, —NO₂, heteroaryl or heteroalkyl; each R_{3b} is independently selected from substituted or unsubstituted lower alkyl, substituted or unsubstituted lower cycloalkyl, phenyl or benzyl; each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
R₆ is H, -L₂-(substituted or unsubstituted alkyl), -L₂-(substituted or unsubstituted cycloalkyl), -L₂-(substituted or unsubstituted alkenyl), -L₂-(substituted or unsubstituted cycloalkenyl), -L₂-(substituted or unsubstituted heterocycloalkyl), -L₂-(substituted or unsubstituted heteroaryl), or -L₂-(substituted or unsubstituted aryl), where L₂ is a band, —O—, —S—, —S(═O)—, —S(═O)₂—, —C(═O)—, —CH(OH)—, -(substituted or unsubstituted C₁-C₆ alkyl), or -(substituted or unsubstituted C₂-C₆, alkenyl);
R₇ is L₃-X-L₄-G₁, wherein, L₃ is a bond, substituted or unsubstituted alkyl, substituted or unsubstituted cycloalkyl, substituted or unsubstituted alkenyl, substituted or unsubstituted alkynyl substituted or unsubstituted aryl, substituted or unsubstituted heteroaryl, substituted or unsubstituted heterocycloalkyl; X is a bond, —O—, —C(═O)—, —CR₉(OR₉)—, —S—, —S(═O)—, —S(═O)₂—, —NR₉—, —NR₉C(═O)—, —C(═O)NR₉—, —S(═O)₂NR₉—, —NR₉S(═O)₂—, —OC(═O)NR₉—, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₄ is a bond, substituted or unsubstituted alkyl, substituted or unsubstituted cycloalkyl substituted or unsubstituted alkenyl, substituted or unsubstituted alkynyl; G₁ is H tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, —CN, —N(R₉)₂, —N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O, —OC(═O)NH—, —NHC(═O)—, —C(═O)NH, —C(═O)O, or —OC(═O)—; or G₁ is W-G₅, where W is a substituted or unsubstituted aryl substituted or unsubstituted heterocycloalkyl, or substituted or unsubstituted heteroaryl; and G₅ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl, phenyl or benzyl; each R₉ is independently selected from H, substituted or unsubstituted lower alkyl, substituted or unsubstituted lower cycloalkyl phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, —S(═O)₂R₈, —S(═O)₂NH₂, —C(═O)R₈, —CN, —NO₂, heteroaryl, or heteroalkyl;
R₅ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, —S(═O)₂, —NH—, —C(═O)—, —NHC(═O)O—, —OC(═O)NH—, —NBC(═O)—, NHC(═O)NH—, or —C(═O)NH;
R₁₁ is L₇-L₁₀-G₆; wherein, L₇ is a bond, —C(═O)—, —C(═O)NH—, -(substituted or unsubstituted C₁-C₆ alkyl)-, or (substituted or unsubstituted C₂-C₆ alkenyl)-; L₁₀ is a -(substituted or unsubstituted cycloalkyl)-, (substituted or unsubstituted cycloalkenyl)-, -(substituted or unsubstituted heteroaryl)-, -(substituted or unsubstituted aryl)-, or -(substituted or unsubstituted heterocycloalkyl); G₆ is tetrazolyl, —NHS(═O)₂R₈, —C(═O)NHS(═O)₂R₈, —S(6═O)₂NHC(═O)R₈, —C(—NR₁₀)N(R₈)₂, —NR₉C(—NR₁₀)N(R₉)₂, —NR₉C(═CHR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—; or G₆ is W-G₇, wherein W is a (substituted or unsubstituted cycloalkyl), (substituted or unsubstituted cycloalkenyl), (substituted or unsubstituted aryl), (substituted or unsubstituted heterocycloalkyl), or a (substituted or unsubstituted heteroaryl); and G₇ is, tetrazolyl, —NHS(═O)₂R₈, S(═O)₂N(R₉), —OH, —C(═O)CF₃, —C(═O)NHS(═O)₂R₈, —S(═O)₂NHC(O)R₈, —N(R₉)₂, —C(═NR₁₀)N(R₈)₂, —NR₉C(═NR₁₀)N(R₉)₂, —NR₉C(═CHR₁₀)N(R₉)₂, —C(═O)N(R₉)₂, -L₅-(substituted or unsubstituted alkyl), -L₅-(substituted or unsubstituted alkenyl), -L₅-(substituted or unsubstituted heteroaryl), -L₅-(substituted or unsubstituted heterocycloalkyl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—; or —OC(═O)—;
R₁₂ is L₈-L₉-R₁₃, wherein L₈ is a bond, (substituted or unsubstituted C₁-C₆ alkyl), or (substituted or unsubstituted C₂-C₄ alkenyl); L₉ is a bond, —O—, —S—, —S(═O)—, —S(═O)₂—, —NH—, —C(═O)—, —NHC(═O)O—, —OC(═O)NH—, —NHC(═O)NH—, —OC(═O)O—, —NHC(═O)—, —C(═)NH—, —C(═O)O—, or —OC(═O)—; R₁₃ is H, (substituted or unsubstituted C₁-C₆ alkyl), (substituted or unsubstituted C₃-C₆ cycloalkyl), (substituted or unsubstituted aryl), (substituted or unsubstituted heteroaryl), or (substituted or unsubstituted heterocycloalkyl); or R₇ and R₁₂ can together form a 4 to 8-membered heterocyclic ring;

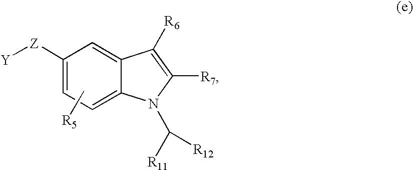

wherein, Z is selected from —NR₁C(═O)O—, —NR₁C(═O)NR₁—, —CR₁═N—N—, wherein each R₁ is independently H, CF₃, or an optionally substituted, lower alkyl;
Y is H, —CO₂H, tetrazolyl, —NHS(═O)₂R_{3B}, S(═O)₂N(R₄)₂, —OH, —OR_{3B}, —C(═O)(C₁-C₅ fluoroalkyl), —C(═O)NHS(═O)₂R_{3b}, —S(═O)₂NHC(O)R₄, CN, N(R₄)₂, —N(R₄)C(═O)R₄, —C(═NR₃)N(R₄)₂, —NR₄C(═NR₃)N(R₄)₂, —NR₄C(═CHR₃)N(R₄)₂, —C(═O)NR₄C(═NR₃)N(R₄)₂, —C(═O)NR₄C(═CHR₃)N(R₄)₂, —CO₂R_{3b}, —C(═O)R₄, —C(═O)N(R₄)₂, —SR_{3b}, —S(═O)R_{3b}, —S(═O)₂R_{3b}, -L₁-(substituted or unsubstituted alkyl), -L₁-(substituted or unsubstituted alkenyl), -L₁-(substituted or unsubstituted alkynyl), -L₁-(substituted or unsubstituted cycloalkyl), -L₁-(substituted or unsubstituted heterocycloalkyl), -L₁-(substituted or unsubstituted heteroaryl), -L₁-(substituted or unsubstituted aryl), -L₁-C(═NR₄)N(R₄)₂, -L₁-NR₄C(═NR₄)N(R₄)₂, or -L₁-NR₄C(═CHR₃)N(R₄)₂;
where L₁ is a bond, a substituted or unsubstituted alkyl, substituted or unsubstituted alkenyl, substituted, or unsubstituted alkynyl, a substituted or unsubstituted heterocycloalkyl, a substituted or unsubstituted heteroaryl, a substituted or unsubstituted cycloalkyl, a substituted or unsubstituted heteroalkyl, substituted or unsubstituted heteroalkenyl, a substituted or unsubstituted heteroalkynyl, or substituted or unsubstituted aryl;
where each substituent is -LₛRₛ, wherein each Lₛ is independently selected from a bond, —O—, —C(═O)—, —S—, —S(═O)—, —S(═O)₂—, —NHC(═O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, C₁-C₆ alkyl, C₂-C₆ alkenyl, —C₁-C₆ fluoroalkyl, heteroaryl, aryl, or heterocycle; and each Rₛ is independently selected, from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl;
each R₃ is independently selected from H, —S(═O)₂R₈, —S(═O)₂NH₂, —C(═O)R₈, —CN, —NO₂, heteroaryl, or heteroalkyl; each R_{3b} is independently selected from substituted or unsubstituted lower alkyl, substituted or unsubstituted lower cycloalkyl, phenyl or benzyl; each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
R₆ is H, -L₂-(substituted or unsubstituted alkyl), -L₂-(substituted or unsubstituted cycloalkyl), -L₂-(substituted or unsubstituted alkenyl), -L₂-(substituted or unsubstituted cycloalkenyl), -L₂-(substituted or unsubstituted heterocycloalkyl), -L₂-(substituted or unsubstituted heteroaryl), or -L₂-(substituted or unsubstituted aryl), where L₂ is a bond, —O—, —S—, —S(═O)—, —S(═O)₂—, —C(═O)—, —CH(OH)—, -(substituted or unsubstituted C₁-C₆ alkyl), or -(substituted or unsubstituted C₂-C₆ alkenyl);
R₇ is L₃-X-L₄-G₁, wherein, L₃ is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycloalkyl; X is a bond, —O—, —C(═O)—, —CR₉(OR₉)—, —S—, —S(═O)—, —S(═O)₂—, —NR₉—, —NR₉C(═O)—, —C(═O)NR₉—, —S(═O)₂NR₉—, —NR₉S(═O)₂—, —OC(═O)NR₉—, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₄ is a bond, substituted or unsubstituted alkyl, substituted or unsubstituted cycloalkyl, substituted or unsubstituted alkenyl, substituted, or unsubstituted alkynyl; G₁ is H, tetrazolyl —NHS(═O)₂R₈, S(═O)₂N(R₉)₂, —OR₉, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —(═O)R₉, —C(═O)N(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O, —OC(═O)NH—, —NHC(═O)—, —C(═O)NH—, —C(═O)O—, or —OC(═O)—; or G₁ is W-G₅, where W is a substituted or unsubstituted aryl, substituted or unsubstituted heterocycloalkyl, or substituted or unsubstituted heteroaryl; and G₅ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl, phenyl or benzyl; each R₉ is independently selected from H, substituted or unsubstituted lower alkyl, substituted or unsubstituted lower cycloalkyl phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, . . . S(═O)₂R₈, —S(═O)₂NH₂—C(═O)R₈, —CN, —NO₂, heteroaryl, or heteroalkyl;
R₅ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, —S(═O)₂—, —NH—, —C(═O)—, —NHC(═O)O—, —OC(═O)NH—, —NHC(═O)—, —NHC(═O)NH—, or —C(═O)NH;
R₁₁ is L₇-L₁₀-G₆; wherein L₇ is a bond, —O—, —S—, —S(═O)—, —S(═O)₂, —NH—, —C(═O)—, —C(═O)NH—, —NHC(═O)—, (substituted or unsubstituted C₁-C₆ alkyl), or (substituted or unsubstituted C₂-C₆ alkenyl); L₅ is a bond, (substituted or unsubstituted alkyl), (substituted or unsubstituted cycloalkyl), (substituted or unsubstituted cycloalkenyl), (substituted or unsubstituted heteroaryl), (substituted or unsubstituted aryl), or (substituted or unsubstituted heterocycloalkyl); and G₆ is H, —CN, —SCN, —N₃, —NO₂, halogen, —OR₉, —C(═O)CF₃, —C(═O)R₉, —SR₈, —S(═O)R₈, —S(═O)₂R₈, —N(R₉)₂, tetrazolyl, —NHS(═O)₂R₈, —S(═O)₂N(R₉)₂, —C(═O)NHS(═O)₂R₈, —S(═O)₂NHC(═O)R₉, —C(═NR₁₀)N(R₉)₂, —NR₉C(—NR₁₀)N(R₉)₂, —NR₉C(═CHR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(═O)O—, —NHC(═O)NH—, —OC(═O)O—, —OC(═O)NH, —NHC(═O)—, —C(═O)NH—, —C(═O)O—, or —OC(═O)—; or G₆ is W-G₇, wherein W is (substituted or unsubstituted cycloalkyl), (substituted or unsubstituted cycloalkenyl), (substituted or unsubstituted aryl), (substituted or unsubstituted heterocycloalkyl) or a (substituted or unsubstituted heteroaryl); and G₇ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is —NH—, —NHC(═O)O—, —NHC(═O)NH—, —OC(═O)O—, —OC(═O)NH—, —NHC(═O)—, —C(═O)NH, —C(═O)O—, or —OC(═O)—;
R₁₂ is L₈-L₉-R₁₃, wherein L₈ is a bond, (substituted or unsubstituted C₁-C₆ alkyl), or (substituted or unsubstituted C₂-C₄ alkenyl); L₉ is a bond, —O—, —S(═O)—, —S(═O)₂—, —NH—, —C(═O)—, —NHC(═O)O—, —OC(═O)NH—, —NHC(═O)NH—, —OC(═O)O—, —NHC(═O)—, —C(═O)NH—, —C(═O)O—, or —OC(═O)—; R₁₃ is H, (substituted or unsubstituted C₁-C₆ alkyl), (substituted or unsubstituted C₃-C₆ cycloalkyl), (substituted or unsubstituted aryl), (substituted or unsubstituted heteroaryl), or (substituted or unsubstituted heterocycloalkyl): or R₇ and R₁₂ can together form a 4 to 8-membered heterocyclic ring;

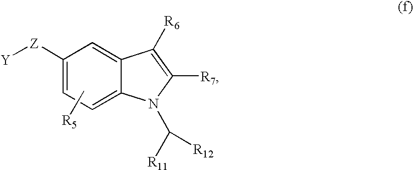

wherein, Z is selected from —N(R₁)—, —S(═O)ₘ—, —CR₁═CR₁—, —C≡C—, —C(R₁)₂[C(R₂)₂]ₙ—, —[C(R₂)₂]ₙC(R₁)₂O—, —OC(R₁)₂[C(R₂)₂]ₙ—, —[C(R₂)₂]ₙC(R₁)₂S(O)ₘ—, —S(O)ₘC(R₁)₂[C(R₂)₂]ₙ—, —[C(R₂)₂]ₙC(R₁)₂NR₁—, —NR₁C(R₁)₂[C(R₂)₂]ₙ—, —[C(R₂)₂]ₙO[C(R₁)₂]ₙ—, —[C(R₁)₂]ₙO[C(R₂)₂]ₙ—, —C(═O)NR₂—, —NR₂C(═O)—, —NR₂C(═O)O—, —OC(═O)NR₂—, —S(═O)₂NR₂—, —CR₁═N—N—, —NR₂C(═O)NR₂—, —OC(═O)O—, S(═O)₂NR₂, or —NR₂S(═O)₂—, wherein each R₁ is independently H, —CF₃, or an optionally substituted lower alkyl; or two R₁ on the same carbon may join to form an oxo (═O); each R₂ is independently H, (═O), —OMe, —CF₃, or an optionally substituted lower alkyl; or two R₂ on the same carbon may join to form an oxo (═O); m is 0, 1 or 2; each n is independently 0, 1, 2, or 3;
Y is —C(═O)NHS(═O)₂R_{3b}, —S(═O)₂NHC(═O)R₄, —C(═O)NR₄C(═NR₃)N(R₄)₂, —C(O)NR₄C(═CHR₃)N(R₄)₂, —C(═O)N(R₄)₂, -L₁-(substituted or unsubstituted heterocycloalkyl), -L₁-C(═NR₄)N(R₄)₂, -L₁-NR₄C(═NR₃)N(R₄)₂, -L₁-NR₄C(═CHR₃)N(R₄)₂, provided that when the heteroatom is directly bound to Z, the heterocycloalkyl is substituted;
where L₁ is a bond, a substituted or unsubstituted alkyl, substituted or unsubstituted alkenyl, or substituted or unsubstituted alkynyl, a substituted or unsubstituted heterocycloalkyl, a substituted or unsubstituted heteroaryl, a substituted or unsubstituted cycloalkyl, a substituted or unsubstituted heteroalkyl, substituted or unsubstituted heteroalkenyl, or a substituted or unsubstituted heteroalkynyl:
where each substituent is -LₛRₛ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, C₁-C₆ alkyl, C₂-C₆ alkenyl, —C₁-C₆ fluoroalkyl, heteroaryl aryl, or heterocycle: and each Rₛ is independently selected from H, halogen, —N(R₄)₂, —CN, —NO₃, N₃, —S(═O)₂NH₂, lower alkyl, lower cycloalkyl, —C₁-C₆ fluoroalkyl, heteroaryl or heteroalkyl;
each R₃ is independently selected from H, —S(═O)₂R₈, —S(═O)₂NH₂, —C(═O)R₈, —CN, —NO₂; heteroaryl, or heteroalkyl; each R_{3b} is independently selected from substituted or unsubstituted lower alkyl substituted or unsubstituted lower cycloalkyl, phenyl or benzyl; each R₄ is independently selected from H, substituted or unsubstituted lower alkyl, substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7, or 8-membered heterocyclic ring;
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycloalkyl), L₂-(substituted or unsubstituted heteroaryl), or L₂-(substituted or unsubstituted aryl), where L₂ is a bond, —O—, —S—, —S(═O)—, —S(═O)₂—, —C(═O)—, —CH(OH)—, -(substituted or unsubstituted C₁-C₆ alkyl), or -(substituted or unsubstituted C₂-C₆ alkenyl);
R₇ is L₃-X-L₄-G₃, wherein, L₃ is a bond, substituted or unsubstituted alkyl, substituted or unsubstituted cycloalkyl, substituted or unsubstituted alkenyl, substituted or unsubstituted alkynyl, substituted or unsubstituted aryl, substituted or unsubstituted heteroaryl, substituted or unsubstituted heterocycloalkyl; X is a bond, —O—, —C(═O)—, —CR₉(OR₉)—, —S—, —S(═O)—, —S(═O)₂—, —NR₉—, —NR₉C(═O)—, —C(═O)NR₉—, —S(═O)₂NR₉—, —NR₉S(═O)₂—, —OC(═O)NR₉—, —NR₉C(═O)O—, —CH═NO—, —ON═CH—, —NR₉C(═O)NR₉—, heteroaryl, aryl, —NR₉C(═NR₁₀)NR₉—, —NR₉C(═NR₁₀)—, —C(═NR₁₀)NR₉—, —OC(═NR₁₀)—, or —C(═NR₁₀)O—; L₄ is a bond, substituted, or unsubstituted alkyl, substituted or unsubstituted cycloalkyl, substituted or unsubstituted alkenyl, substituted or unsubstituted alkynyl; G₁ is H, tetrazolyl, —NHS(═O)₂R₈, S(═O)₂N(R₉)₂, —OR₉, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O, —OC(═O)NH—, —NHC(═O)—, —C(═O)NH—, —C(═O)O—, or —OC(═O)—; or G₁ is W-G₅, where W is a substituted or unsubstituted aryl, substituted or unsubstituted heterocycloalkyl, or substituted or unsubstituted heteroaryl; and G₅ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, or —S(═O)₂R₈; each R₈ is independently selected from substituted or unsubstituted lower alkyl, substituted or unsubstituted lower cycloalkyl, phenyl or benzyl; each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring and each R₁₀ is independently selected from H, —S(O)₂R₈, —S(═O)₂NH₂, —C(O)R₈, —CN, —NO₂, heteroaryl, or heteroalkyl;
R₅ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, S(═O)₂—, —NH—, —C(═O)—, —NHC(═O)O—, —OC(═O)NH—, —NHC(═O)—, —NHC(═O)NH—, or —C(═O)NH—;
R₁₁ is L₇₋L₁₀₋G₆; wherein L₇ is a bond, —O—, —S—, —S(═O)—, —S(═O)₂—, —NH—, —C(═O)—, —C(═O)NH—, —NHC(═O)—, (substituted or unsubstituted C₁-C₆ alkyl), or (substituted or unsubstituted C₂-C₆ alkenyl); L₁₀ is a bond, (substituted or unsubstituted alkyl), (substituted or unsubstituted cycloalkyl), (substituted or unsubstituted cycloalkenyl), (substituted or unsubstituted heteroaryl), (substituted or unsubstituted aryl), or (substituted of unsubstituted heterocycloalkyl); and G₆ is H, —CN, —SCN, —N₃, —NO₂, halogen, —OR₉, —C(═O)CF₃, —C(═O)R₉, —SR₈, —S(═O)R₈, S(═O)₂R₈, —N(R₉)₂, tetrazolyl —NHS(═O)₂R₈, —S(═O)₂N(R₉)₂, —C(═O)NHS(═O)₂R₈, —S(═O)₂NHC(═O)R₉, —C(═NR₁₀)N(R₉)₂, —NR₉C(═NR₁₀)N(R₉)₂, —NR₉C(═CHR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(═O)O—, —NHC(═O)NH—, —OC(═O)O—, —OC(═O)NH—, —NHC(═O)—, —C(═O)NH—, —C(═O)O—, or —OC(═O)—; or G₆ is W-G₇, wherein W is (substituted, or unsubstituted cycloalkyl), (substituted or unsubstituted cycloalkenyl), (substituted or unsubstituted aryl), (substituted or unsubstituted heterocycloalkyl) or a (substituted or unsubstituted heteroaryl); and G₇ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is —NH—, —NHC(═O)O—, —NHC(═O)NH—, —OC(═O)O—, —OC(═O)NH—, —NHC(═O)—, —C(═O)NH—, —C(═O)O—, or —OC(═O)—;
R₁₂ is L₈-L₉-R₁₃, wherein L₈ is a bond, (substituted or unsubstituted C₃-C₆ alkyl), or (substituted or unsubstituted C₂-C₄ alkenyl); L₉ is a bond, —O—, —S—, —S(═O)—, S(═O)₂—, —NH—, —C(═O)—, —NHC(═O)O—, —OC(═O)NH—, —NHC(═O)NH—, —OC(═O)O—, —NHC(═O)—, —C(═O)NH—, —C(═O)O—, or —OC(═O)—; R₁₃, is H, (substituted or unsubstituted C₁-C₆ alkyl), (substituted or unsubstituted C₃-C₆ cycloalkyl), (substituted or unsubstituted aryl), (substituted or unsubstituted heteroaryl), or (substituted or unsubstituted heterocycloalkyl); or R₇ and R₁₂ can together form a 4 to 8-membered heterocyclic ring; or

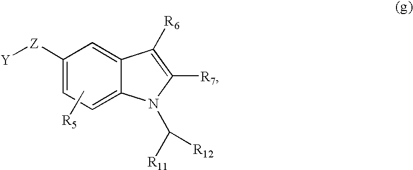

wherein, Z is selected from —N(R₁)—, —S(═O)ₘ—, —CR₁═CR₁—, —C≡C—, —C(R₁)₂[C(R₂)₂]ₙ—, —[C(R₂)₂]ₙC(R₁)₂O—, —OC(R₁)₂[C(R₂)₂]ₙ—, —[C(R₂)₂]ₙC(R₁)₂S(O)ₘ—, —S(O)ₘC(R₁)₂[C(R₂)₂]ₙ—, —[C(R₂)₂]ₙC(R₁)₂NR₁—, —NR₁C(R₁)₂[C(R₂)₂]ₙ—, —[C(R₂)₂]ₙO[C(R₁)₂]ₙ—, —[C(R₁)₂]ₙO[C(R₂)₂]ₙ—, —C(═O)NR₂—, —NR₂C(═O)—, —NR₂C(═O)O—, —OC(═O)NR₂—, —S(═O)₂NR₂—, —CR₁═N—N—, —NR₂C(═O)NR₂—, —OC(═O)O—, S(═O)₂NR₂, or —NR₂S(═O)₂—, wherein each R₁ is independently h, —CF₃, or art optionally substituted lower alkyl, or two R₁ groups on the same carbon may join to form an oxo (═O); and each R₂ is independently H, —OH, —OMe, —CF₃, or an optionally substituted lower alkyl; or two R₂ on the same carbon may join to form an oxo (═O); m is 0, 1 or 2; each ti is independently 0, 1, 2, or 3;
Y is H, —CO₂H, tetrazolyl, —NHS(═O)₂R_{3b}, —S(═O)₂N(R₄)₂, —OH, —OR_{3b}, —C(═O)(C₁-C₅ fluoroalkyl), —C(═O)NHS(═O)₂R_{3b}, —S(═O)₂NHC(O)R₄, —CN, —N(R₄)₂, —N(R₄)C(═O)R₄, —C(═NR₃)N(R₄)₂, —NR₄C(═NR₃)H(R₄)₂, —NR₄C(═CHR₃)N(R₄)₂, —C(═O)NR₄C(═NR₃)N(R₄)₂, —C(O)NR₄C(═CHR₃)N(R₄)₂, —CO₂R_{3b}, —C(═O)R₄, —C(═O)N(R₄)₂, —SR_{3b}, —S(═O)R_{3b}, —S(═O)₂R_{3b}, -L₁-(substituted or unsubstituted alkyl), -L₁-(substituted or unsubstituted alkenyl), -L₁-(substituted or unsubstituted alkynyl), -L₁-(substituted or unsubstituted cycloalkyl), -L₁-(substituted or unsubstituted heterocycloalkyl), -L₁-(substituted or Unsubstituted heteroaryl), -L₁-(substituted or unsubstituted aryl) or -L₁-C(═NR₄)N(R₄)₂, -L₁-NR₄C(═NR₄)N(R₄)₂, -L₁-NR₄C(═CHR₃)N(R₄)₂;
where L₁ is a bond, a substituted or unsubstituted alkyl, substituted or unsubstituted alkenyl, substituted or unsubstituted alkynyl, a substituted or unsubstituted heterocycloalkyl, a substituted or unsubstituted heteroaryl, a substituted or unsubstituted cycloalkyl, a substituted or unsubstituted heteroalkyl, substituted or unsubstituted heteroalkenyl, a substituted or unsubstituted heteroalkynyl, or substituted or unsubstituted aryl;
where each substituent is -LₛRₛ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, C₁-C₆ alkyl, C₂-C₆ alkenyl, C₁-C₆ fluoroalkyl, heteroaryl, aryl, or heterocycle; and each Rₛ is independently selected from H, halogen, —N(R₄)₂, —CN, —NO₂, N₃, —S(═O)₂NH₂, lower alkyl, lower cycloalkyl, —C₁-C₆ fluoroalkyl, heteroaryl, or heteroalkyl;
each R₃ is independently selected from H, —S(═O)₂R₈, —S(═O)₂NH₂, —C(O)R₈, —CN, —NO₂, heteroaryl, or heteroalkyl; each R_{3b} is independently selected from substituted or unsubstituted lower alkyl, substituted or unsubstituted lower cycloalkyl, phenyl or benzyl; each R₄ is independently selected front H, substituted or unsubstituted lower alkyl substituted or unsubstituted lower cycloalkyl, phenyl or benzyl; or two R₄ groups can together form a 5-, 6-, 7-, or 8-membered heterocyclic ring; or R_{3b} and R₄ can together form a 5-, 6-, 7-, or 8-membered heterocyclic ring;
R₆ is H, -L₂-(substituted or unsubstituted alkyl), -L₂-(substituted or unsubstituted cycloalkyl), -L₂-(substituted or unsubstituted alkenyl), -L₂-(substituted or unsubstituted cycloalkenyl), -L₂-(substituted or unsubstituted heterocycloalkyl), -L₂-(substituted or unsubstituted heteroaryl), or -L₂-(substituted or unsubstituted aryl); where L₂ is a bond, —O—, —S—, —S(═O)—, —S(═O)₂—, —C(═O)—, —CH(OH)—, -(substituted or unsubstituted C₁-C₆ alkyl), or -(substituted or unsubstituted C₂-C₆ alkenyl);
R₇ is H or substituted or unsubstituted alkyl;
R₅ is H, halogen, —N₃, —CN, —ONO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, —S(═O)₂—, —NH—, —C(═O)—, —NHC(═O)O—, —OC(═O)NH—, —NHC(═O)—, —NHC(═O)NH—, or —C(O)NH—;
R₁₁ is a (substituted or unsubstituted heteroaryl) or (substituted or unsubstituted heterocycloalkyl); and
R₁₂ is L₈-L₉-R₁₃, wherein L₈ is a bond, (substituted or unsubstituted C₁-C₆ alkyl), or (substituted or unsubstituted C₂-C₄ alkenyl); L₉ is a bond, —O—, —S—, —S(═O)—, S(═O)₂—, —NH—, —C(═O)—, —NHC(═O)O—, —OC(═O)NH—, —NHC(═O)NH—, —OC(═O)O—, —NHC(═O)—, —C(═O)NH—, —C(═O)O—, or —OC(═O)—; R₁₃, is H, (substituted or unsubstituted C₁-C₆ alkyl), (substituted or unsubstituted C₃-C₆ cycloalkyl), (substituted or unsubstituted aryl), (substituted or unsubstituted heteroaryl), or (substituted or unsubstituted heterocycloalkyl).

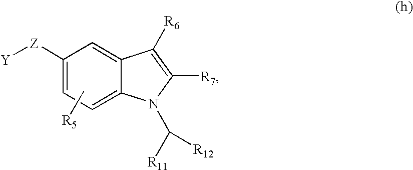

Z is selected from [C(R₁)₂]ₘ[C(R₂)₂]ₙ, [C(R₂)₂]ₙ[C(R₁)₂]ₘO, O[C(R₁)₂]ₘ[C(R₂)₂]ₙ, [C(R₂)₂]ₙO[C(R₁)₂]ₙ, or [C(R₁)₂]ₙO[C(R₂)₂]ₙ, wherein each R₁ is independently H, CF₃, or an optionally substituted lower alkyl; or two R₂ on the same carbon may join to form an oxo (═O); and each R₂ is independently H, OH, OMe, CF₃, or an optionally substituted lower alkyl; or two R₂ on the same carbon may join to form an oxo (═O); m is 0, 1 or 2; each n is independently 0, 1, 2, or 3;
Y is H or -(substituted or unsubstituted and); or -(substituted or unsubstituted heteroaryl);
where each substituent on Y or Z is -LₛRₛ, wherein each Lₛ is independently selected from a bond, —O—, —C(═O)—, —S—, —S(═O)—, —S(═O)₂—, —NHC(O)—, —C(O)NH—, S(═O)₂NH—, —NHS(═O)₂, —OC(O)NH—, —NHC(O)O—, —OC(O)O—, —NHC(O)NH—, —C(O)O—, —OC(O)—, substituted or unsubstituted C₁-C₆ alkyl, C₂-C₆ alkenyl, —C₁-C₆ fluoroalkyl, substituted or unsubstituted heteroaryl, substituted or unsubstituted carbocyclic aryl, or substituted or unsubstituted heterocycloalkyl; and each Rₛ is independently selected from H, halogen, —N(R₄)₂, —CN, —NO₂, N₃, —S(═O)₂NH₂, substituted or unsubstituted lower alkyl, substituted or unsubstituted lower cycloalkyl —C₁-C₆ fluoroalkyl, substituted or unsubstituted aryl substituted or unsubstituted heteroaryl or substituted or unsubstituted heteroalkyl;
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl). L₂-(substituted or unsubstituted heterocycloalkyl), L₂-(substituted or unsubstituted heteroaryl), or L₂-(substituted or unsubstituted aryl), where L₂ is a bond, O, S, —S(═O), —S(═O)₂, C(O), —CH(OH), -(substituted or unsubstituted C₁-C₆ alkyl), or -(substituted or unsubstituted C₂-C₆ alkenyl);
R₇ is L₃-X-L₄-G₁, wherein,
L₃ is a substituted or unsubstituted alkyl;
X is a bond, O, —C(═O)—, —C(═O)O—, —CR₉(OR₉)—, —S—, —S(═O)—, —S(═O)₂—, —NR₉—, —NR₉C(═O)—, —C(═O)NR₉—, —NR₉C(═O)NR₉—;
L₄ is a bond, or a substituted or unsubstituted alkyl;
G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted heteroalkyl), -L₅-(substituted or unsubstituted alkenyl), -L₅-(substituted or unsubstituted heteroaryl), or -L₅-(substituted or unsubstituted aryl), wherein L₅ is —OC(O)O—, —NHC(O)NH—, —NHC(O)O, —O(O)CNH—, —NHC(O), —C(O)NH, —C(O)O, or —OC(O);
or G₁ is W-G₅, where W is a substituted or unsubstituted carbocyclic aryl, substituted or unsubstituted heterocycloalkyl or substituted or unsubstituted heteroaryl; and G₅ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, —CN, —N(R₉)₂, —N(R₉)₂, —N(R₉)C(O)R₉, —C(═NR₁₀)N(R₉)₂, —NR₉C(═NR₁₀)N(R₉)₂, —NR₉C(═CR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈;
each R₈ is independently selected from substituted or unsubstituted lower alkyl, substituted or unsubstituted lower cycloalkyl, substituted or unsubstituted phenyl or substituted or unsubstituted benzyl;
each R₉ is independently selected from H, substituted or unsubstituted lower alkyl, substituted or unsubstituted lower cycloalkyl, substituted or unsubstituted phenyl or substituted or unsubstituted benzyl; or two R₉ groups can together form a 5-, 6-, 7-, or 8-membered heterocyclic ring; or R₈ and R₉ can together form a 5-, 6-, 7-, or 8-membered heterocyclic ring and
each R₁₀ is independently selected from H, —S(═O)₂R₈, —S(═O)₂NH₂, —C(O)R₈, —CN, —NO₂, heteroaryl, or heteroalkyl;
R₅ is H, halogen, substituted or unsubstituted C₁-C₆ alkyl, substituted or unsubstituted O—C₁-C₆ alkyl:
R₁₁ is L₇-L₁₀-G₆, wherein L₇ is a bond; L₁₀ is a (substituted or unsubstituted heteroaryl), (substituted or unsubstituted aryl), or (substituted or unsubstituted heterocycloalkyl);
G₆ is OR₉, —C(═O)R₉, —C(O)OR₉, —SR₈, —S(═O)R₈, —S(═O)₂R₈, N(R₉)₂, tetrazolyl, —NHS(═O)₂R₈, —S(═O)₂N(R₉)₂, —C(O)NHS(═O)₂R₈, —S(═O)₂NHC(O)R₉, —C(═O)N(R₉)₂, NR₉C(O)R₉, C(R₉)₂C(═O)N(R₉)₂, —C(═NR₁₀)N(R₉)₂, —NR₉C(═NR₁₀)N(R₉)₂, —NR₉C(═CHR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted carbocyclic aryl), wherein L₅ is —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
or G₆ is W-G₇, wherein W is (substituted or unsubstituted heterocycloalkyl), (substituted or unsubstituted aryl) or a (substituted or unsubstituted heteroaryl) and G₇ is H, halogen, CN, NO₂, N₃, CF₃, OCF₃, C₁-C₆ alkyl, C₃-C₆ cycloalkyl, —C₁-C₆ fluoroalkyl, tetrazolyl, —NHS(O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is a bond, —O—, C(═O), S, S(═O), S(═O)₂, —NH, —NHC(O)O, —NHC(O)NH—, —OC(O)O—, —OC(O)NH—, —NHC(O), —C(O)NH, —C(O)O, or —OC(O);
R₁₂ is H, (substituted or unsubstituted C₁-C₆ alkyl), (substituted or unsubstituted C₃-C₆ cycloalkyl);

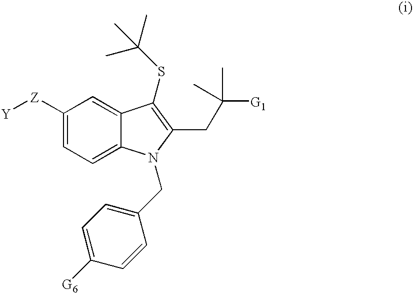

wherein,
Z is [C(R₂)₂]ₙC(R₁)₂O, wherein
each R₁ is independently H, —CF₃, or an optionally substituted lower alkyl; or two R₁ groups on the same carbon may join to form an oxo (═O); each R₂ is independently H, —OH, —OMe, —CF₃, or an optionally substituted lower alkyl; or two R₂ groups on the same carbon may join to form an oxo (═O); n is 0, 1, 2, or 3;
Y is -(substituted or unsubstituted aryl); or -(substituted or unsubstituted heteroaryl);
where each substituent on Y or Z is LₛRₛ, wherein each Lₛ is independently selected from a bond, —O—, —C(═O)—, —S—, —S(═O)—, —S(═O)₂—, —NHC(O)—, —C(═O)NH—, S(═O)₂NH—, —NHS(O)₂, —OC(═O)NH—, —NHC(═O)O—, —OC(═O)O—, —NHC(═O)NH—, —C(═O)O—, —OC(═O)—, substituted or unsubstituted C₁-C₆ alkyl, C₂-C₆ alkenyl, —C₁-C₆ fluoroalkyl, substituted or unsubstituted heteroaryl, substituted or unsubstituted aryl, or substituted or unsubstituted heterocycloalkyl; and each Rₛ is independently selected from H, halogen, —N(R₄)₂, —CN, —NO₂, N₃, —S(═O)₂NH₂, substituted or unsubstituted lower alkyl, substituted or unsubstituted lower cycloalkyl, —C₁-C₆ fluoroalkyl, substituted or unsubstituted aryl, substituted or unsubstituted heteroaryl, or substituted or unsubstituted heteroalkyl;
G₁ is H, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—; or G₁ is W-G₅, where W is a substituted or unsubstituted aryl substituted or unsubstituted heterocycloalkyl or substituted or unsubstituted heteroaryl and G₅ is H, tetrazolyl, —NHS(═O)₂R₈, S(═O)₂N(R₉)₂, OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —(═O)R₉, —C(═O)N(R₉)₂, —SR₈, —S(═O)R₈, or —S(═O)₂R₈;
each R₈ is independently selected from substituted or unsubstituted lower alkyl, substituted or unsubstituted lower cycloalkyl substituted or unsubstituted phenyl or substituted or unsubstituted benzyl;
each R₉ is independently selected from H, substituted or unsubstituted lower alkyl, substituted or unsubstituted lower cycloalkyl substituted or unsubstituted phenyl or substituted or unsubstituted benzyl; or
two R₉ groups can together form a 5-, 6-, 7-, or 8-membered heterocyclic ring; or
R₈ and R₉ can together form a 5-, 6-, 7-, or 8-membered heterocyclic ring;
each R₁₀ is independently selected from H, S(═O)₂R₈, —S(═O)₂NH₂—C(O)R₈, —CN, —NO₂, heteroaryl, or heteroalkyl;
G₆ is W-G₇, wherein W is (substituted or unsubstituted heterocycloalkyl), (substituted or unsubstituted aryl) or a (substituted, or unsubstituted heteroaryl); and
G₇ is H, halogen, CN, NO₂, N₃, CF₃, OCF₂, C₁-C₆ alkyl C₁-C₆ heteroalkyl C₃-C₆ cycloalkyl, —C₁-C₆ fluoroalkyl, tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is a bond, —O—, C(═O)—, —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
or solvate, or pharmaceutically acceptable salt, or a pharmaceutically acceptable prodrug thereof.

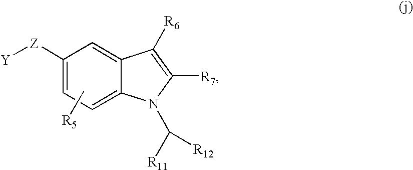

wherein,
Z is selected from —C(R₁)₂[C(R₂)₂]ₙ—, —[C(R₂)₂]ₙC(R₁)₂O—, —OC(R₁)₂[C(R₂)₂]ₙ—, —[(R₂)₂]ₙC(R₁)₂S(O)ₘ—, —S(O)ₘC(R₁)₂[C(R₂)₂]ₙ—, —[C(R₂)₂]ₙO[C(R₁)₂]ₙ—, or —[C(R₁)₂]ₙO[C(R₂)₂]ₙ—; each R₁ is independently H, CF₃, or an optionally substituted lower alkyl; or two R₁ on the same carbon may join to form an oxo (═O); each R₂ is independently H, OH, OMe, CF₃, or an optionally substituted lower alkyl; or two R₂ on the same carbon may join to form an oxo (═O); m is 0, 1 or 2; each n is independently 0, 1, 2, or 3;
Y is H, —CO₂H, tetrazolyl, (substituted, or unsubstituted alkyl), (substituted or unsubstituted heterocycloalkyl group), (substituted or unsubstituted heteroaryl), or (substituted or unsubstituted aryl);
R₆ is H, L₂-(substituted or unsubstituted alkyl), L₂-(substituted or unsubstituted cycloalkyl), L₂-(substituted or unsubstituted alkenyl), L₂-(substituted or unsubstituted cycloalkenyl), L₂-(substituted or unsubstituted heterocycloalkyl group), L₂-(substituted or unsubstituted heteroaryl), or L₂-(substituted or unsubstituted aryl); L₂ is a bond, —O—, —S—, —S(═O)—, —S(═O)₂—, —C(═O)—, —CH(OH)—, -(substituted or unsubstituted C₁-C₆ alkyl), or -(substituted or unsubstituted C₂-C₆ alkenyl);
R₇ is H or lower alkyl;
R₅ is H, halogen, —N₃, —CN, NO₂, -L₆-(substituted or unsubstituted C₁-C₆ alkyl), -L₆-(substituted or unsubstituted C₂-C₆ alkenyl), -L₆-(substituted or unsubstituted heteroaryl), or -L₆-(substituted or unsubstituted aryl), wherein L₆ is a bond, —O—, —S—, —S(═O)—, —S(═O)₂—, —NH—, —C(═O)—, —NHC(═O)O—, —OC(═O)NH—, —NHC(═O)—, —NHC(═O)NH—, or —C(═O)NH—;
R₁₁ is L₇-L₁₀-G₆; wherein
L₇ is a bond; L₁₀ is a (substituted or unsubstituted heteroaryl), (substituted or unsubstituted aryl), or (substituted or unsubstituted heterocycloalkyl group);
G₆ is H, —CN, —SCN, —N₃, —NO₂, halogen, —OR₉, —C(═O)CF₃: —C(═O)R₉, —SR₈, —S(═O)R₈, —S(═O)₂R₈, —N(R₉)₂, tetrazolyl, —NHS(═O)₂R₈, —S(═O)₂N(R₉)₂, —C(═O)NHS(═O)₂R₈, —S(═O)₂NHC(O)R₉, —C(═NR₁₀)N(R₉)₂, —NR₉C(═NR₁₀)N(R₉)₂, —NR₉C(═CHR₁₀)N(R₉)₂, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —NHC(═O)O—, —NHC(═O)NH—, —OC(═O)O—, —OC(═O)NH—, —NHC(═O)—, —C(═O)NH—, —C(═O)O—, or —OC(═O)—; or
G₆ is W-G₇, wherein W is (substituted or unsubstituted cycloalkyl), (substituted or unsubstituted cycloalkenyl), (substituted or unsubstituted aryl), (substituted or unsubstituted heterocycloalkyl group) or a (substituted or unsubstituted heteroaryl), and G₇ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl group), or -L₅-(substituted or unsubstituted aryl), wherein L₅ is —NH—, —NHC(═O)O—, —NHC(═O)NH—, —OC(═O)O—, —OC(═O)NH—, —NHC(═O)—, —C(═O)NH—, —C(═O)O—, or —OC(═O)—;
R₁₂ is L₃-X-L₄-G₁, wherein,
L₃ is a bond, lower alkyl, or alkenyl; X is a bond; L₄ is a bond, lower alkyl, alkenyl, or alkynyl;
G₁ is tetrazolyl, —NHS(═O)₂R₈, S(═O)₂N(R₉)₂, —OH, —OR₈, —C(═O)CF₃, —C(═O)NHS(═O)₂R₈, —S(═O)₂NHC(O)R₉, —CN, —N(R₉)₂, —N(R₉)C(═O)R₉, —C(═NR₁₀)N(R₉)₂, —NR₉C(═NR₁₀)N(R₉)₂, —NR₉C(═CHR₁₀)N(R₉)₂, —C(═O)NR₉C(═NR₁₀)N(R₉)₂, —C(═O)NR₉C(═CHR₁₀)N(R₉)₂, —CO₂R₉, —C(═O)R₉, —C(═O)N(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—;
each R₈ is independently selected from substituted or unsubstituted lower alkyl, substituted or unsubstituted lower cycloalkyl phenyl or benzyl; each R₉ is independently selected from H, substituted or unsubstituted lower alkyl, substituted or unsubstituted lower cycloalkyl, phenyl or benzyl; or two R₉ groups can together form a 5-, 6-, 7-, or 8-membered heterocyclic ring; or R₈ and R₉ can together form a 5-, 6-, 7-, or 8-membered heterocyclic ring; and
each R₁₀ is independently selected from H, —S(═O)₂R₈, —S(═O)₂NH₂—C(O)R₈, —CN, —NO₂, heteroaryl, or heteroalkyl; or solvate, or pharmaceutically acceptable salt, or a pharmaceutically acceptable prodrug thereof.

Any of the aforementioned FLAP inhibitors may be used in the combination compositions and therapies described herein.

### Combination Compounds

In one aspect, combination compounds of Formula (I), pharmaceutically acceptable salts, pharmaceutically acceptable N-oxides, pharmaceutically acceptable prodrugs, and pharmaceutically acceptable solvates thereof, antagonize or inhibit FLAP and/or modulate NO levels in vivo, and are used to treat patients suffering from NO-dependent, NO-mediated, leukotriene-dependent or leukotriene mediated conditions or diseases, including, but not limited to, hypertension, asthma, myocardial infarction, cancer, gastrointestinal lesions, gastrointestinal injury, and inflammatory conditions;

Aₓ-L-B Formula (I)

wherein, A is a moiety that in the form A′ (which includes A-X, A-H, A⁻, or A⁺) is an NO-modulator, or a moiety that upon activation-reaction produces NO, or a moiety selected from —NO₂ or —ONO₂, wherein X is COOH, CONH₂, OH, NH₂, halogen, SH, or CH₃; x is 0, 1, 2, 3, 4, 5, 6, 7, 8, 9, 10, 11, 12, 13, 14, 15, 16, 17, 17, 19, or 20.
Suitable NO-modulators include nitroprusside, nitroglycerin, isosorbide mononitrate, and isosorbide dinitrate; compounds that stimulate endogenous NO or elevate levels of endogenous endothelium-derived relaxing factor (EDRF) in vivo or are substrates for nitric oxide synthase, including arginine, homoarginine, and N-hydroxy-arginine, including their nitrosated and nitrosylated analogs (e.g. nitrosated arginine, nitrosylated arginine, nitrosated N-hydroxy-arginine, nitrosylated N-hydroxy-arginine, nitrosated homoarginine and nitrosylated homoarginine); precursors of arginine and/or physiologically acceptable salts thereof, including, for example, citrulline, ornithine, glutamine, lysine, polypeptides comprising at least one of these amino acids; inhibitors of the enzyme arginase (e.g., N-hydroxy-L-arginine and 2(S)-amino-6-boronohexanoic acid) and the substrates for nitric oxide synthase; cytokines, adenosine, bradykinin, calreticulin, bisacodyl, phenolphthalein; molsidomine; 3-morpholinosydnonimine (SIN-1); 1,2,3,4-oxatriazolium, 5-amino-3-(3,4-di-chlorophenyl)-chloride (GEA 3162); 1,2,3,4-oxatriazolium, 5-amino-3-(3-chloro-2-methyl-phenyl)chloride (GEA502-4); 1,2,3,4-oxatriazolium, 3-(3-chloro-2-methylphenyl)5-[[[cyanomethylamino-]carbonyl]amino]-hydroxide inner salt (GEA 5583); S-nitroso-N-acetyl-D,L-penicillamine (SNAP); Glyco-SNAP-1; Glyco-SNAP-2,2,2′-(hydroxynitrosohydrazono)bis-ethanamine (NOC-18) and (+/−)-(E)-4-ethyl-2-[(E)-hydroxyimino]-5-nitro-3-hexenamide (NOR-3); 1-[(4′,5′-bis(carboxymethoxy)-2′-nitrophenyl)methoxy]-2-oxo-3,3,diethyl-1-triazene dipotassium salt (CNO-4): [1-(4′,5′-bis(carbomethoxy)-2′-nitrophenyl)methoxy]-2-oxo-3,3-diethyl-1-triazine diacetoxymethyl ester (CNO-5); b diethylamine-NO (DEA/NO), IPA/NO, spermine-NO(SPER/NO), sulfite-NO (SULFI/NO), OXI/NO, and DETA/NO; cicletanine; sulfonamide NO donors GEA 3268, (1,2,3,4-oxatriazolium, 3-(3-chloro-2-methylphenyl)-5-[[(4-methoxyphenyl)-sulfonyl]amino]-, hydroxide inner salt) and GEA 5145, (1,2,3,4-oxatriazolium, 3-(3-chloro-2-methylphenyl)5-[(methylsulfonyl)amino]-, hydroxide inner salt); sulfonamide GEA 3175; S-nitrosothiols; nitrites; nitrates, N-oxo-N-nitrosoamines, SPM 3672, SPM 5185, SPM 5186 and analogs thereof. In one embodiment, all statins are excluded as potential NO modulators.
Aₓ-L-B includes structures wherein A is a moiety that by itself produces or provides one or more than one NO molecule per A group, including, by way of example only, trinitroglycerin, which can produce or provide 3 NO molecules per A group, in one aspect, the “L” group contains multiple A groups, each of which produces one or more NO molecules per A group. In another aspect, B is bound with one or more -L-Aₓ groups, wherein, each A group produces one or more NO molecules per A group. In another aspect, the compound of Formula (I) contains more than one “A” group and each “A” group is the same, different, or some “A” groups are the same and some “A” groups are different; in embodiments where the multiple “A” groups are different, then, the compound of Formula (I) releases, provides or produces cleaved “A” moieties by the same or different cleavage/activation pathways. In one aspect, the compound of Formula (I) contains more than one “A” group, and each “A” group has the same or different “L” groups,
L is a bond or a moiety that chemically links the A and B moieties, and is cleaved (in a single step or in multiple steps; chemically, enzymatically, biologically, photochemically, electrochemically, sonochemically, and by other activation, means described herein) to produce -L′-A and -B′, -L′-B and -A′, or -L′, -A′ and -B′. In one aspect, L is attached to a suitable position on the FLAP inhibitor such that it is readily cleaved in vivo. For example, in the case where the FLAP inhibitor contains a carboxylic acid, L is coupled to the acid group of the FLAP inhibitor through an ester or amide bond. In the case that the FLAP inhibitor contains an amino group, L is attached through an amide, carbonate or carbamate group. In the case that the FLAP inhibitor contains an alcohol or phenol group, L is attached through an ether, ester, carbonate or carbamate group, in one aspect, L is a moiety that is cleavable in a single step or in multiple steps.
In one aspect, compounds of Formula (I) (Aₓ-L-B) are cleaved into a FLAP inhibitor and an NO modulator only or predominantly at a specific site in vivo; by way of example only, Aₓ-L-B remains intact upon administration, but is cleaved into a FLAP inhibitor and an NO modulator by a specific enzyme (e.g., a targeted esterase) that is produced only or predominantly by a particular cell type. In one aspect, the FLAP inhibitor is released from Aₓ-L-B (or from -L-B) by one site-specific pathway (single or multiple enzymes) and the NO modulator released, from Aₓ-L-B (or Aₓ-L) by another site-specific pathway (single or multiple enzymes); alternatively, both, the FLAP inhibitor and the NO modulator are released from Aₓ-L-B by the same site-specific pathway (single or multiple enzymes).
In one aspect, a residue, or portion, of L remains with “A” or “B” after a cleavage step; further cleavage steps remove further portions or residues of the L group still remaining with “A” or “B.” However, a residue or portion of “L” remains with “A” or “B” after cleavage or further reaction provided that the “A” group can act as an NO modulator and the “B” group can act as a FLAP inhibitor.
B is a moiety that in the form B′ (including B-X, B-H, B⁻ or B⁺) is a FLAP inhibitor.
In one aspect, A or B include a carboxylic acid group, and are coupled to an -L-B group or -L-A group via an ester or amide bond. In another aspect, A or B includes an amino group, and are coupled to an -L-B group or -L-A group via an amide, carbonate or carbamate bond. In another aspect, A or B contains an alkoxy or phenoxy moiety, and is coupled to an -L-B group or -L-A group via these moieties.
In one aspect, the FLAP inhibitor is selected from Abbott-81834; Abbott-93178; MK-591 (MK0591), MK-886, BAY X 1005, BAY Y 1015, ETH603, ETH615, ETH647, WAY-121520, and WY 50295. In another aspect, the FLAP inhibitor is selected from compounds described, in U.S. Pat. Nos. 4,929,626, 4,970,215, 5,081,138, 5,095,031, 5,204,344, 5,126,354, 5,221,678, 5,229,516, 5,272,145, 5,283,252, 5,288,743, 5,292,769, 5,304,563, 5,399,699, 5,459,150, 5,512,581, 5,597,833, 5,668,146, 5,668,150, 5,691,351, 5,714,488, 5,783,586, 5,795,900, and 5,843,968. In another aspect, the FLAP inhibitor is selected from any of FLAP compounds (a)-(h) described elsewhere herein. In yet another aspect, the FLAP inhibitor is selected from compounds described in U.S. patent publication no. 2007/0123522, U.S. patent publication no. 2007/0105866, U.S. patent publication no. 2007/0219206, U.S. patent publication no. 2007/0225285, U.S. patent publication no. 2007/0244128, International patent publication no, WO 07/056021, International patent publication no. WO 07/056,220, international patent publication no. WO 07/056338, International patent publication no, WO 07/047,207, each of which is herein incorporated by reference.
In one aspect, L is a bond and a NO modulator as described herein is directly coupled to a FLAP inhibitor as described herein.

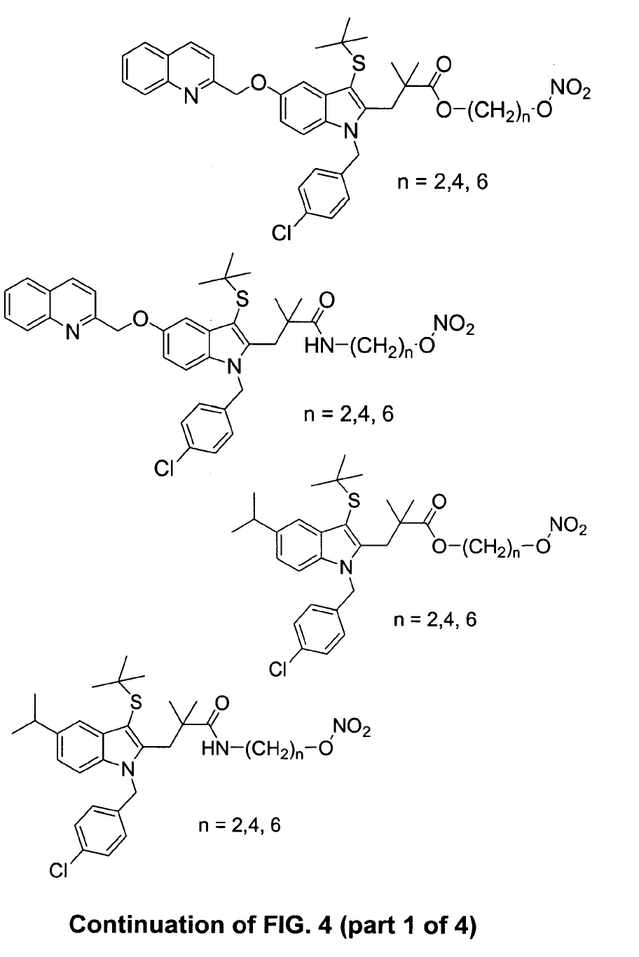

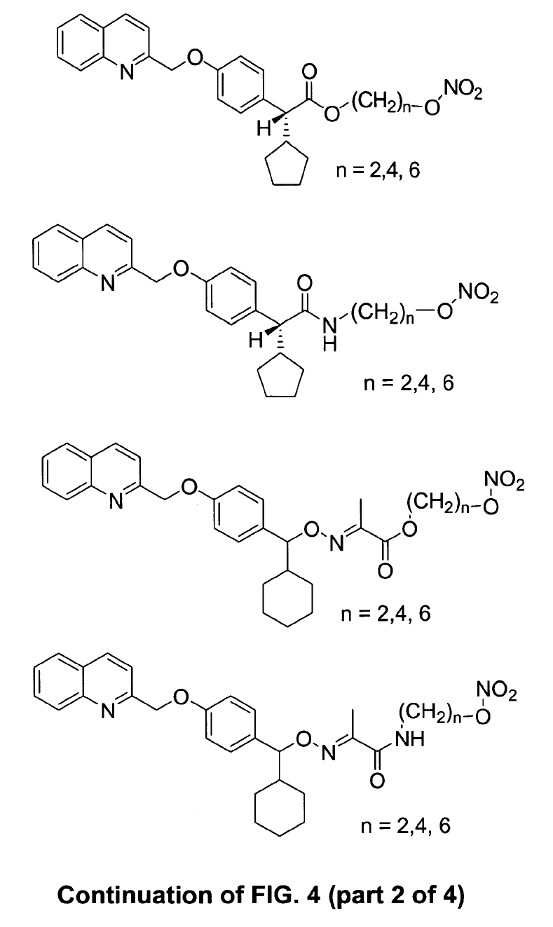

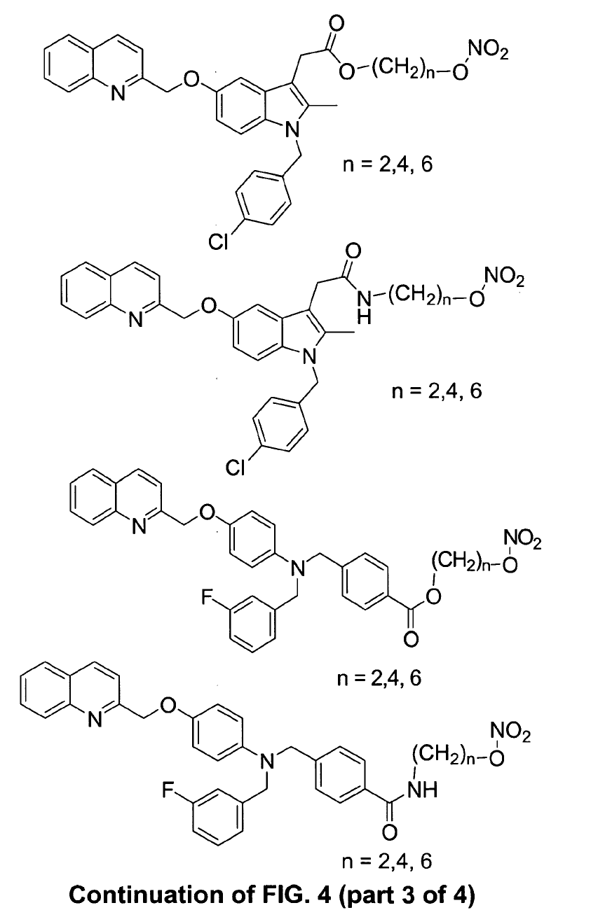

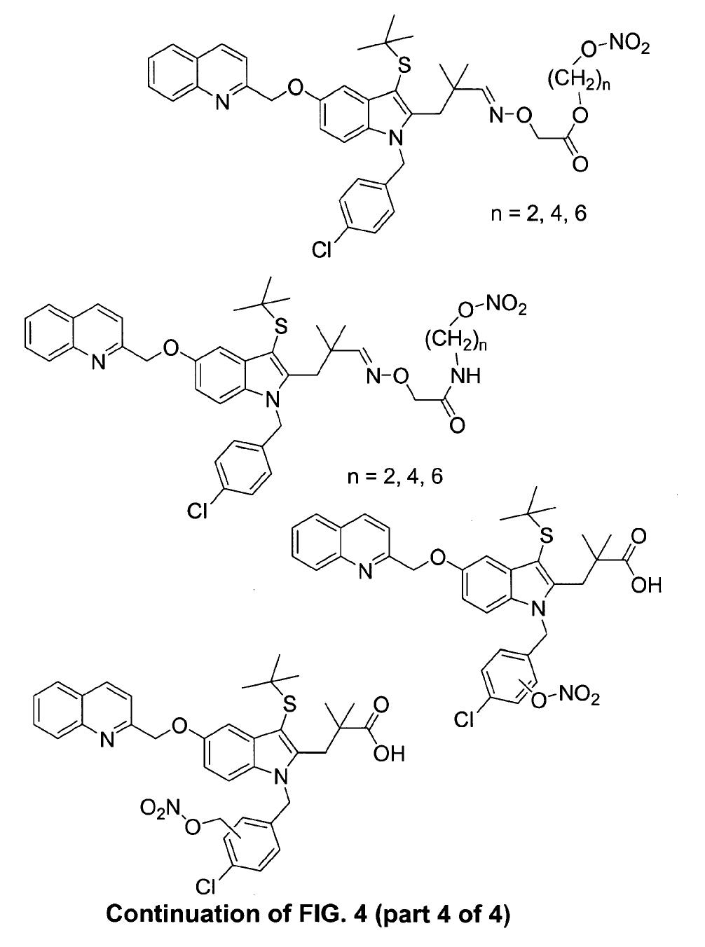

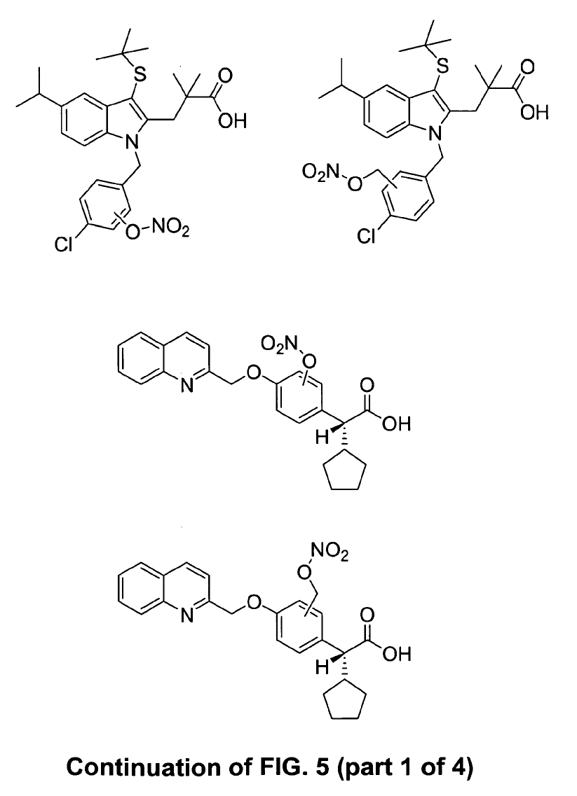

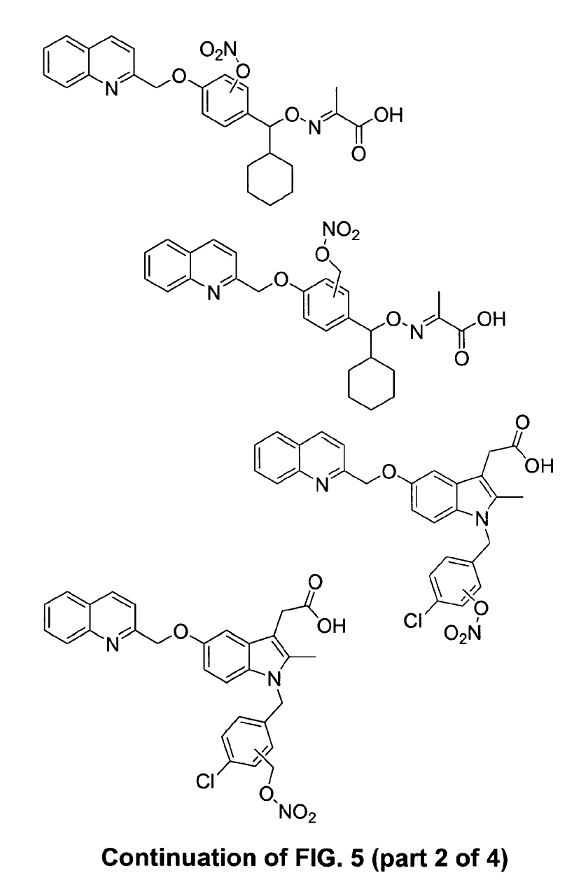

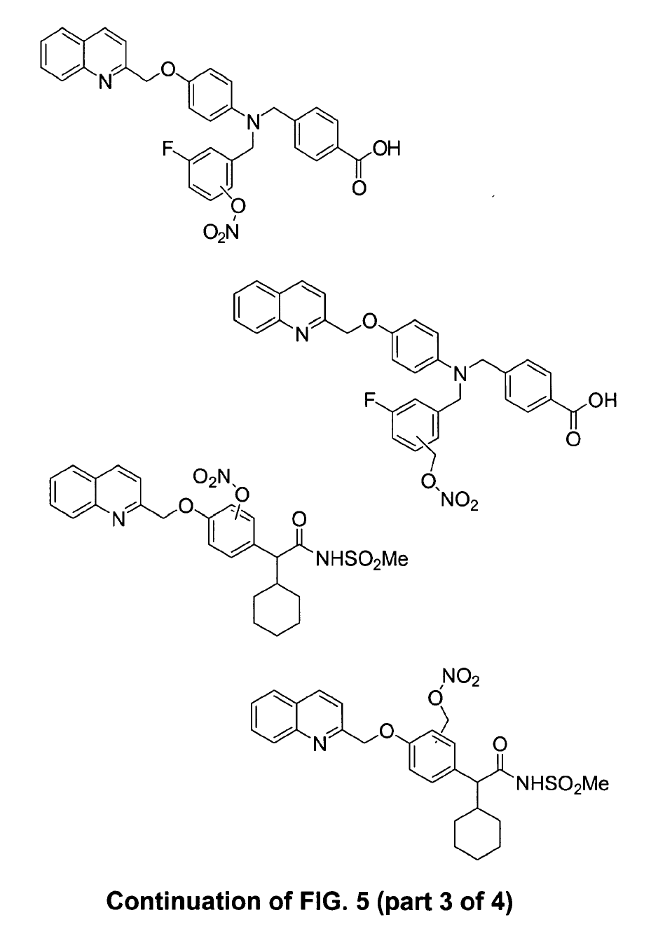

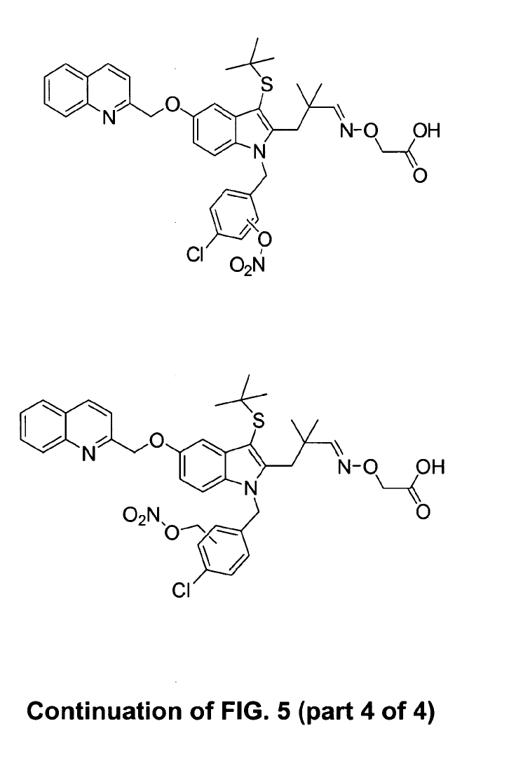

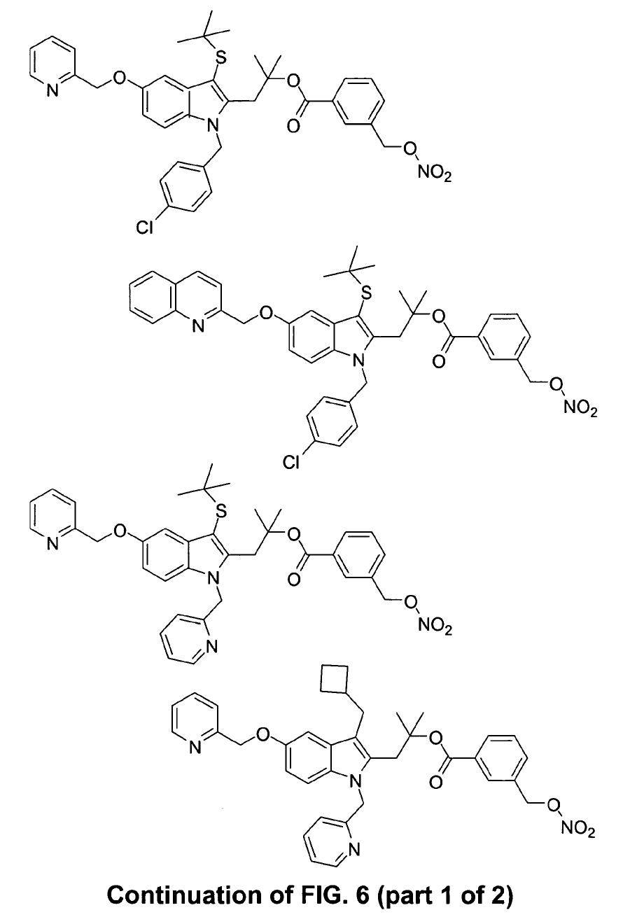

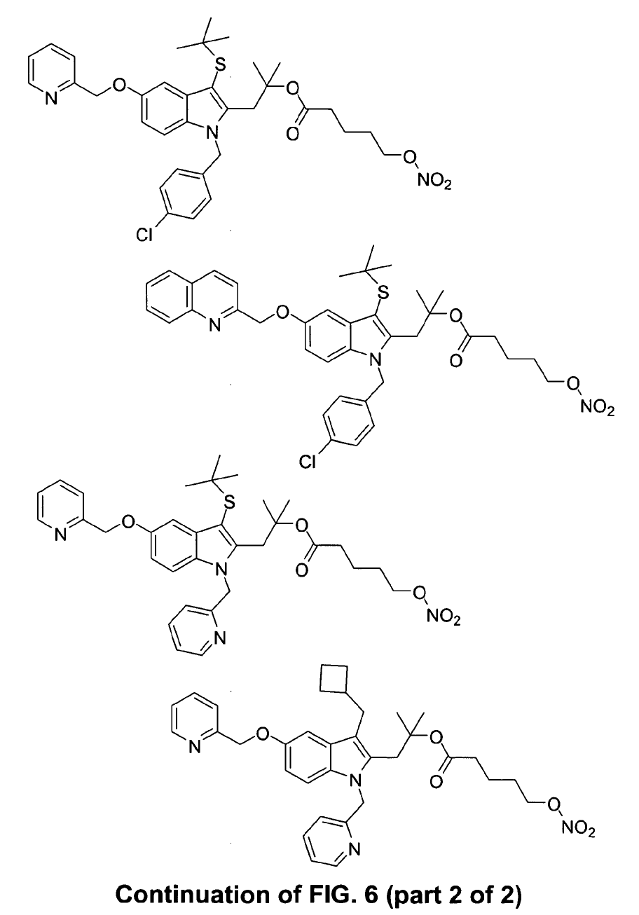

In one aspect, compounds of Formula (I) include those shown in FIG. 4, FIG. 5. FIG. 6, and in Table 1:

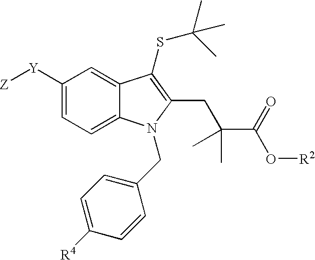

| Com- | | | | |
|---|---|---|---|---|
| pound | | | | |
| # | Y—Z | R⁴ | R² | M + H |
| 1-1b | isopropyl | Cl | 6-nitrooxy- | See Exp |
| | | | hexyl | |
| | | | (L-A) | |
| 1-2b | pyridin-2- | 6-methoxy- | 6-nitrooxy- | See Exp |
| | ylmethoxy | pyridin-3-yl | hexyl | |
| | | | (L-A) | |
| 1-3b | (S)-1-acetyl- | 6-methoxy- | 6-nitrooxy- | See Exp |
| | 2,3-dihydro-1H- | pyridazin-3-yl | hexyl | |
| | indol-2- | | (L-A) | |
| | ylmethoxy | | | |
| 1-4b | (S)-1-isobutyryl- | Cl | 6-nitrooxy- | See Exp |
| | pyrrolidin-2- | | hexyl | |
| | ylmethoxy | | (L-A) | |
| 1-5b | 5-methyl-pyridin- | 5-nitrooxymethyl- | H | 669 |
| | 2-ylmethoxy | pyridin-2-yl | | |
| | | (L-A) | | |
| 1-6b | pyridin-2- | 6-ethoxy- | 2-hydroxy-3- | 744 |
| | ylmethoxy | pyridin-3-yl | nitrooxy-propyl | |
| | | | (L-A) | |
| 1-7b | pyridin-2- | 6-ethoxy- | isosorbide-5- | 797 |
| | ylmethoxy | pyridin-3-yl | mononitrate | |
| | | | (L-A) | |

Compounds in Table 3 are named:

3-[3-tert-Butylsulfanyl-1-(4-chloro-benzyl)-5-isopropyl-1H-indol-2-yl]-2,2-dimethyl-propionic acid 6-nitrooxy-hexyl ester (Compound 1-1b); 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid 6-nitrooxy-hexyl ester (Compound 1-2b); 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid 6-nitrooxy-hexyl ester (Compound 1-3b); 3-[3-tert-Butylsulfanyl-1-(4-chloro-benzyl)-5-((S)-1-isobutyryl-pyrrolidin-2-ylmethoxy)-1H-indol-2-yl]-2,2-dimethyl-propionic acid 6-nitrooxy-hexyl ester (Compound 1-4b); 3-{3-tert-Butylsulfanyl-5-(5-methyl-pyridin-2-ylmethoxy)-1-[4-(5-nitrooxymethyl-pyridin-2-yl)-benzyl]-1H-indol-2-yl}-2,2-dimethyl-propionic acid (Compound 1-5b); 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2-hydroxy-3-nitrooxy-propyl ester (Compound 1-6b); and 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid isosorbide-5-mononitrate ester (Compound 1-7b).

### Synthesis of the Combination Compounds

Combination compounds of Formula (I) are synthesized, using methods described herein, or using methods known in the art in combination with methods described herein, in addition, solvents, temperatures and other reaction conditions presented herein may vary according to the practice and knowledge of those of skill in the art.

The starting material used for the synthesis of the compounds of Formula (I) and compounds having the structures described in the prior section are obtained, from commercial sources, such as Aldrich Chemical Co. (Milwaukee, Wis.), Sigma Chemical Co. (St. Louis, Mo.), or the starting materials can be synthesized. The compounds described herein, and other related compounds having different substituents are synthesized using techniques and materials, such as described, for example, in March, ADVANCED ORGANIC CHEMISTRY 4^{th} Ed., (Wiley 1992); Carey and Sundberg, ADVANCED ORGANIC CHEMISTRY 4^{th} Ed. Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compound as disclosed herein may be derived from known reactions in the field, and the reactions may be modified by the use of appropriate reagents and conditions. As a guide the following synthetic methods may be utilized.

Formation of Covalent Linkages by Reaction of an Electrophile with a Nucleophile

The compounds described herein can be modified using various electrophiles or nucleophiles to form new functional groups or substituents. Table 2 entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophile groups and nucleophilic groups.

TABLE 2
Examples of Covalent Linkages and Precursors Thereof

| Covalent Linkage Product | Electrophile | Nucleophile |
|---|---|---|
| Carboxamides | Activated esters | amines/anilines |
| Carboxamides | acyl azides | amines/anilines |
| Carboxamides | acyl halides | amines/anilines |
| Esters | acyl halides | alcohols/phenols |
| Esters | acyl nitriles | alcohols/phenols |
| Carboxamides | acyl nitriles | amines/anilines |
| Imines | Aldehydes | amines/anilines |
| Hydrazones | aldehydes or ketones | Hydrazines |
| Oximes | aldehydes or ketones | Hydroxylamines |
| Alkyl amines | alkyl halides | amines/anilines |
| Esters | alkyl halides | carboxylic acids |
| Thioethers | alkyl halides | Thiols |
| Ethers | alkyl halides | alcohols/phenols |
| Thioethers | alkyl sulfonates | Thiols |
| Esters | alkyl sulfonates | carboxylic acids |
| Ethers | alkyl sulfonates | alcohols/phenols |
| Esters | Anhydrides | alcohols/phenols |
| Carboxamides | Anhydrides | amines/anilines |
| Thiophenols | aryl halides | Thiols |
| Aryl amines | aryl halides | Amines |
| Thioethers | Azindines | Thiols |
| Boronate esters | Boronates | Glycols |
| Carboxamides | carboxylic acids | amines/anilines |
| Esters | carboxylic acids | Alcohols |
| hydrazines | Hydrazides | carboxylic acids |
| N-acylureas or Anhydrides | carbodiimides | carboxylic acids |
| Esters | diazoalkanes | carboxylic acids |
| Thioethers | Epoxides | Thiols |
| Thioethers | haloacetamides | Thiols |
| Ammotriazines | halotriazines | amines/anilines |
| Triazinyl ethers | halotriazines | alcohols/phenols |
| Amidines | imido esters | amines/anilines |
| Ureas | Isocyanates | amines/anilines |
| Urethanes | Isocyanates | alcohols/phenols |
| Thioureas | isothiocyanates | amines/anilines |
| Thioethers | Maleimides | Thiols |
| Phosphite esters | phosphoramidites | Alcohols |
| Silyl ethers | silyl halides | Alcohols |
| Alkyl amines | sulfonate esters | amines/anilines |
| Thioethers | sulfonate esters | Thiols |
| Esters | sulfonate esters | carboxylic acids |
| Ethers | sulfonate esters | Alcohols |
| Sulfonamides | sulfonyl halides | amines/anilines |
| Sulfonate esters | sulfonyl halides | phenols/alcohols |

### Use of Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd⁰-catalyzed reaction, in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typically blocking/protecting groups include:

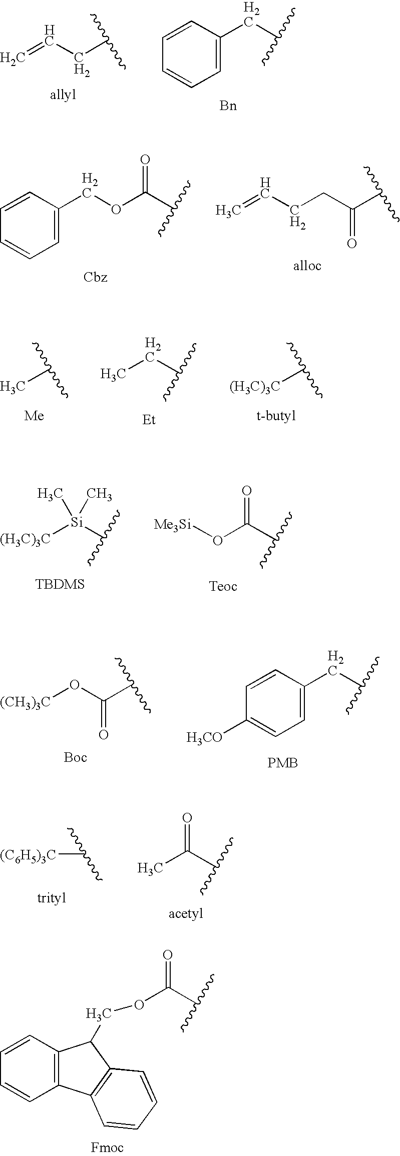

Other protecting groups, plus a detailed description of techniques applicable to the creation of protecting groups and their removal are described in Greene and Wuts, Protective Groups in Organic Synthesis, 3rd Ed., John Wiley & Sons, New York, N.Y., 1999, and Kocienski, Protective Groups, Thieme Veriag, New York, N.Y., 1994, which are incorporated herein by reference in their entirety.

### Synthesis of Compounds of Formula (I)

In one aspect, compounds of Formula (I) are synthesized according to reaction Scheme I. Esters of Formula I-5 are prepared as shown in Scheme I starting with carboxylic acids of Formula I-1 (where RCO₂H is a FLAP inhibitor as described herein). Conversion of carboxylic acids of Formula I-1 to the corresponding acid chlorides of Formula I-2 is achieved using standard conditions (for example using SOCl₂). The acid chloride is then reacted with a bromo (or chloro or iodo) alcohol of structure Br-L¹-OH in the presence of a base such as Et₃N in an organic solvent such as tetrahydrofuran. (THF) yielding compounds of Formula I-3. Alternatively, carboxylic acids of Formula I-1 are converted to a metal salt such as sodium salts of Formula I-4 using a base such as NaH in a solvent such as dimethylformamide (DMF). Displacement of a dihalo derivative such as Br-L¹-Br then yields compounds of Formula I-3. Treatment of compounds of Formula I-3 with AgNO₃ in acetonitrile affords the final product of Formula I-5. In one aspect, sodium salts of Formula I-4 are reacted with Br-L¹-ONO₂ to give compounds of Formula I-5.

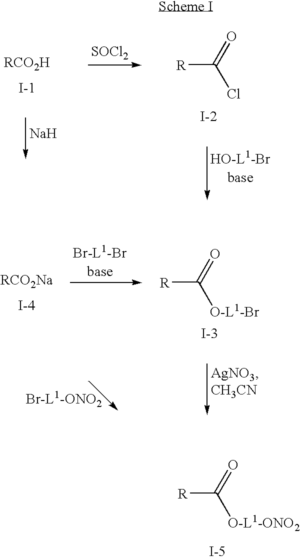

in another aspect, compounds of Formula (I) are synthesized according to reaction Scheme II. Amides of Formula II-3 are prepared using the route described in Scheme II. Acid chlorides of Formula I-2 are coupled with an aminoalcohol of general structure H₂N-L¹-OH to give amides of Formula II-1. Treatment of amides of Formula II-1 with a halogenating agent provides the bromides (or chlorides) of Formula II-2 which are reacted with AgNO₃ in acetonitrile to provide compounds of Formula II-3.

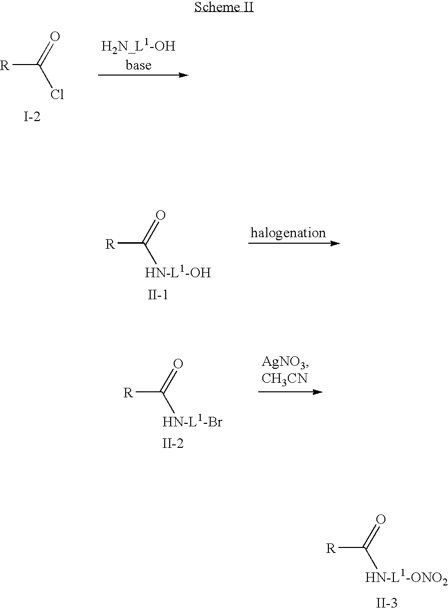

In another aspect, compounds of Formula (I) are synthesized according to reaction Scheme III. Hydroxyl containing compounds (for example alcohols or phenols or N-hydroxyureas) are converted to the corresponding ONO₂ linked derivatives of Formula III-3 as shown in Scheme III. Conversion of the hydroxyl group to the sodium salt, which provides sodium salts of Formula III-1 (where RONa is a sodium salt of a FLAP inhibitor that has a hydroxy moiety as described herein), is achieved using a base such as NaH in a solvent like THF. Sodium salts of Formula III-1 are then reacted with a dibromoderivative such as Br-L¹-Br to yield bromo compounds of Formula III-2. Bromo compounds of Formula III-2 are converted, as described above, to compounds of Formula III-3. Alternatively, compounds of general structure III-1 are treated with a bromo linked NO₃ reagent in a dipolar aprotic solvent such as DMF to afford the desired compound III-3.

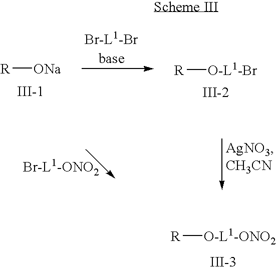

In yet another aspect, compounds of Formula (I) are synthesized according to reaction Scheme IV (where R—OH is a FLAP inhibitor as described herein). Chloro acids of Formula IV-1 are converted into the corresponding nitroesters of Formula IV-2 using AgNO₃ in a solvent such as CH₃CN (see Breschi et al, J. Med. Chem., 2004, 47, p 5597-5600). Coupling of nitroesters of Formula IV-2 with FLAP compounds containing a hydroxy group (—OH) using standard esterification conditions (e.g. DCC and DMAP in THF) then yields a compound of general structure IV-3.

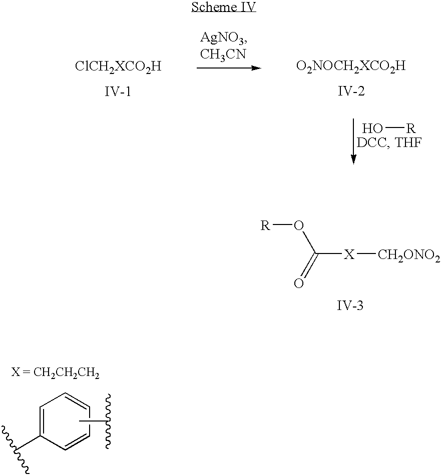

### Further Forms of Compounds

For convenience, the form and other characteristics of the compounds described, in this section and other parts herein, use a single formula, such as “Formula (I),” by way of example. However, the form and other characteristics of the compounds described herein apply equally well to all formulas presented herein that fall within the scope of Formula (I), FLAP inhibitors, NO modulators, as well as to all of the specific compounds that fall within the scope of these general categories.

In one embodiment, compounds of Formula (I) are prepared as a pharmaceutically acceptable salts formed when an acidic proton present in the parent compound either is replaced by a metal ion, for example an alkali metal ion, an alkaline earth ion, or art aluminum ion; or coordinates with an organic base. In another embodiment, the salt forms of the disclosed compounds are prepared using salts of the starting materials or intermediates.

In one embodiment, compounds of Formula (I) are prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Alternatively, compounds of Formula (I) are prepared as a pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric, or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds of Formula (I) can be conveniently prepared or formed during the processes described herein. By way of example only, hydrates of compounds of Formula (I) are conveniently prepared by recrystallization from an aqueous/organic solvent mixture, using organic solvents including, but not limited to, dioxane, tetrahydrofuran, ethanol, ethyl acetate or methanol. In one embodiment, the compounds provided herein exist in unsolvated forms, in another embodiment, the compounds provided herein exist in solvated forms. In general, the solvated forms are considered equivalent to the unsolvated forms for the purposes of the compounds and methods provided herein.

Compounds of Formula (I) include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate. Compounds of Formula (I) also include crystalline forms known as co-crystals.

In one aspect, compounds of Formula (I) in unoxidized form are prepared from N-oxides of compounds of Formula (I) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In one aspect, compounds of Formula (I) are prepared as prodrugs. Prodrugs are generally drug precursors that, following administration to a subject and subsequent absorption, are converted to an active, or a more active species via some process, such, as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound of Formula (I) which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In one aspect, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed Chrom., 6:283-286 (1992); J. Larsen and H. Bundgaard, Int. J. Pharmaceutics. 37, 87 (1987); J. Larsen et al., Int J. Pharmaceutics, 47, 103 (1988); Sinkula et al., J. Pharm. Sci., 64; 181-210 (1975): T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Prodrug derivatives of compounds of Formula (I) are prepared by methods known to those of ordinary skill in the art (e.g., for further details see Saulnier et al., (1994), Bioorganic and Medicinal Chemistry Letters, Vol. 4, p. 1985). By way of example only, in one aspect appropriate prodrugs are prepared by reacting a non-derivatized compound of Formula (I) with a suitable carbamoylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds may be a prodrug for another derivative or active compound.

In some embodiments, sites on the aromatic ring portion of compounds of Formula (I) are susceptible to various metabolic reactions, and therefore incorporation of appropriate substituents on the aromatic ring structures, such as, by way of example only, halogens, or alkyl groups, reduce, minimize or eliminate this metabolic pathway.

In some embodiments, the compounds described herein are labeled isotopically (e.g. with a radioisotope) or by another other means, including, but not limited to, the use of chromophores or fluorescent moieties, bioluminescent labels, or chemiluminescent labels.

In some embodiments, the compounds of Formula (I) possess one or more stereocenters and each center may exist in the R or S configuration. The compounds presented herein include all diastereomeric, enantiomeric, and epimeric forms as well as the appropriate mixtures thereof. In some embodiments, compounds of Formula (I) are prepared as their individual stereoisomers by reacting a racemic mixture of the compound with an optically active resolving agent to form a pair of diastereoisomeric compounds, separating the diastereomers and recovering the optically pure enantiomers. In one embodiment, resolution of enantiomers is carried out using covalent diastereomeric derivatives of the compounds described herein. In another embodiment, resolution of enantiomers is carried out using dissociable complexes (e.g., crystalline diastereomeric salts). Diastereomers have distinct physical properties (e.g., melting points, boiling points, solubilities, reactivity, etc.) and are readily separated by taking advantage of these dissimilarities. In some embodiments, the diastereomers are separated by chiral chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981, herein incorporated by reference in its entirety.

In some embodiments, the compounds provided herein may exist as geometric isomers. The compounds and methods provided herein include all cis, trans, syn, anti, entgegen (E), and zusammen (Z) isomers as well as the appropriate mixtures thereof. In some situations, compounds exist as tautomers. All tautomers are included within the formulas described herein are provided by compounds and methods herein. In additional embodiments of the compounds and methods provided herein, mixtures of enantiomers and/or diastereoisomers, resulting from a single preparative step, combination, or interconversion are also useful for the applications described herein.

### Certain Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg “ADVANCED ORGANIC CHEMISTRY 4^{th} ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed.

An “alkoxy” group refers to a (alkyl)O— group, where alkyl is as defined herein.

An “alkyl” group refers to an aliphatic hydrocarbon group. The alkyl moiety may be a “saturated alkyl” group, which means that it does not contain any units of unsaturation (e.g. carbon-carbon double bonds or carbon-carbon triple bonds). The alkyl moiety may also be an “unsaturated alkyl” moiety, which means that it contains at least one unit of unsaturation. The alkyl moiety, whether saturated or unsaturated, may be branched, straight chain, or cyclic. The point of attachment of an alkyl is at a sp3 carbon atom that is not part of a ring.

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 5 carbon atoms. The alkyl group of the compounds described herein may be designated as “C₁-C₄ alkyl” or similar designations. By way of example only. “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, allyl, but-2-enyl, but-3-enyl, cyclopropylmethyl, cyclobutylmethyl, cyclopentylmethyl, cyclohexylmethyl, and the like. In one aspect, an alkyl is a C₁-C₆ alkyl.

The term “alkylamine” refers to the —N(alkyl)ₓH_{y} group, where x and y are selected from the group x=1, y=1 and x=2, y=0. In some embodiments, when x=2 and y=0, the alkyl groups taken together with the nitrogen atom to which they are attached form a cyclic ring system.

The term “alkenyl” refers to a type of alkyl group in which the first two atoms of the alkyl group form a double bond that is not part of an aromatic group. That is, an alkenyl group begins with the atoms —C(R)═(R)₂, wherein R refers to the remaining portions of the alkenyl group, which may be the same or different. Non-limiting examples of an alkenyl group include —CH═CH₂, —C(CH₃)═CH₂, —CH═CHCH₃ and —C(CH₃)═CHCH₃. The alkenyl moiety may be branched, straight chain, or cyclic (in which case, it would also be known as a “cycloalkenyl” group). In one aspect, an alkenyl is a C₂-C₆alkenyl.

The term “alkynyl” refers to a type of alkyl group in which the first two atoms of the alkyl group form a triple bond. That is, an alkynyl group begins with the atoms —C≡C—R, wherein R refers to the remaining portions of the alkynyl group, which may be the same or different. Non-limiting examples of an alkynyl group include —C≡CH, —C≡CCH₃ and —C≡CCH₂CH₃. In one aspect, an alkynyl is a C₂-C₆alkynyl.

An “amide” is a chemical moiety with formula —C(═O)NHR or —NHC(═O)R, where R is selected from the group consisting of alkyl, cycloalkyl, aryl, heteroaryl (bonded through a ring carbon) and heteroalicyclic (bonded through a ring carbon). In one aspect, an amide is an amino acid or a peptide molecule attached to a compound of Formula (I), a FLAP inhibitor or an NO modulator, thereby forming a prodrug. Any amine, or carboxyl side chain on the compounds described herein can be used to prepare an amide group. Procedures and specific groups to make such amides include those found in, e.g., Greene and Wuts, Protective Groups in Organic Synthesis, 3^{rd} Ed., John Wiley & Sons, New York, N.Y., 1999, which is incorporated herein by reference in its entirety.

The term “aromatic” refers to a planar ring having a delocalized π-electron system containing 4n+2 π electrons, where n is an integer. Aromatic rings can be formed from five, six, seven, eight, nine, ten, or more than ten atoms. Aromatics cart be optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” refers to a ring or ring system where the atoms forming the backbone of the ring are all carbon atoms. The term thus distinguishes carbocyclic from heterocyclic rings in which the ring, backbone contains at least one atom which is different from carbon.

As used herein, the term “aryl” refers to an aromatic ring wherein each, of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, and naphthalenyl. Depending on the structure, an aryl group can be a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. Cycloalkyls may be saturated, or partially unsaturated. Cycloalkyls may be fused with an aromatic ring, and the point of attachment is at a carbon that is not an aromatic ring carbon atom. Cycloalkyl groups include groups having from 3 to 10 ring atoms. A lower cycloalkyl refers to a C₃-C₈ cycloalkyl. Illustrative examples of cycloalkyl groups include, but are not limited to, the following moieties:

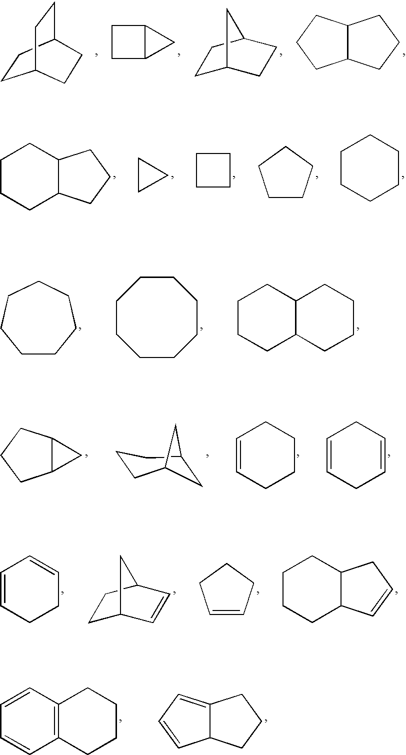

and the like. In some embodiments, cycloalkyl groups are selected from among cyclopropyl, cyclobutyl, cyclopentyl, cyclohexyl, cycloheptyl, and cyclooctyl. Cycloalkyl groups may be substituted or unsubstituted. Depending on the structure, a cycloalkyl group can be a monoradical or a diradical (i.e., an cycloalkylene group, such as, but not limited to, cyclopropan-1,1-diyl, cyclobutan-1,1-diyl, cyclopentan-1,1-diyl, cyclohexan-1,1-diyl, cycloheptan-1,1-diyl, and the like).

The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-diaxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (═O) moieties such as pyrrolidin-2-one.

The terms “heteroaryl” or, alternatively, “heteroaromatic” refers to an aryl group in which one or more ring atoms is a heteroatom, and the heteroatoms are selected from nitrogen, oxygen and sulfur. Illustrative examples of heteroaryl groups include the following moieties:

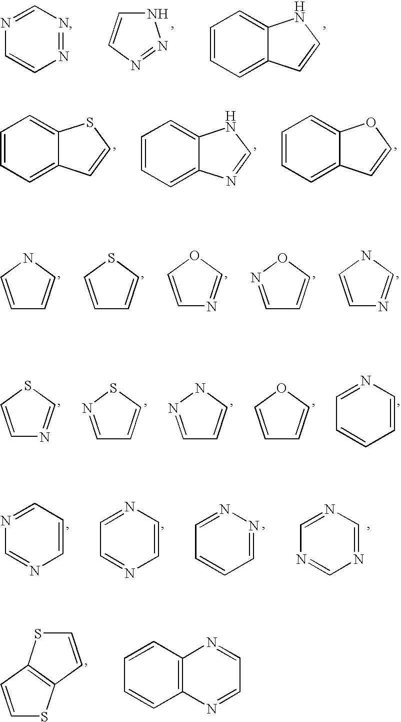

and the like. An N-containing “heteroaromatic” or “heteroaryl” moiety refers to an aromatic group in which at least one of the skeletal atoms of the ring is a nitrogen atom.

A “heteroalicyclic” group or “heterocycloalkyl” refers to a cycloalkyl group that includes at least one heteroatom selected from nitrogen, oxygen and sulfur. The radicals may be fused with an aryl or heteroaryl. Illustrative examples of heterocycloalkyl groups, also referred to as non-aromatic heterocycles, include:

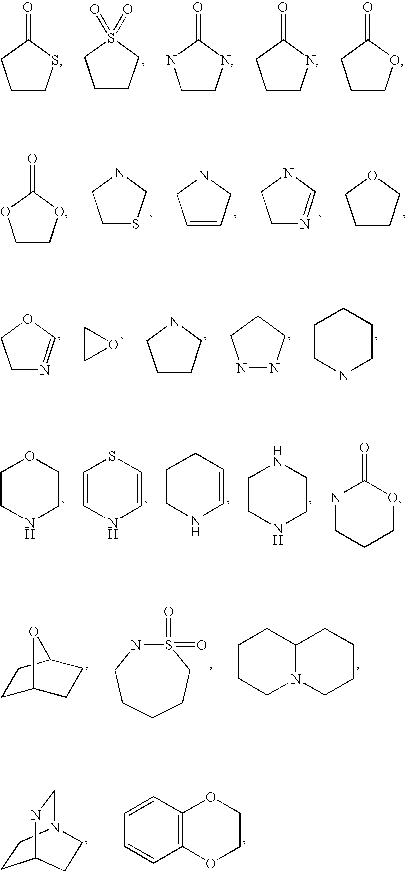

and the like. In some embodiments, the heterocycloalkyl is selected from pyrrolidinyl, tetrahydrofuranyl, tetrahydrothienyl, tetrahydropyranyl, tetrahydrothiopyranyl, piperidinyl, morpholinyl, thiomorpholinyl, piperazinyl, and indolinyl. The term heteroalicyclic also includes all ring forms of the carbohydrates, including but not limited to the monosaccharides, the disaccharides and the oligosaccharides. In one aspect, a heterocycloalkyl is a C₂-C₈heterocycloalkyl. A C₂-C₈heterocycloalkyl refers to the number of carbon atoms that make up the heterocycloalkyl, excluding the heteroatoms that are present.

The term “ester” refers to a chemical moiety with formula —COOR, when R is selected from the group consisting of alkyl, cycloalkyl, aryl, heteroaryl (bonded through a ring carbon) and heteroalicyclic (bonded through a ring carbon). Any hydroxy, or carboxyl side chain on the compounds described herein can be esterified. Examples of procedures and specific groups to make such esters can be found in sources such as Greene and Wuts, Protective Groups in Organic Synthesis, 3^{rd} Ed., John Wiley & Sons, New York, N.Y., 1999, which is incorporated herein by reference in its entirety.

The term “halo” or, alternatively, “halogen” or “halide” means fluoro, chloro, bromo or iodo.

The term “haloalkyl” refers to an alkyl group in which one or more hydrogen atoms are replaced by one or more halide atoms. In one aspect, a haloalkyl is a C₂-C₆haloalkyl.

The term “haloalkenyl” refers to an alkenyl group in which one or more hydrogen atoms are replaced by one or more halide atoms. In one aspect, a haloalkenyl is a C₂-C₆haloalkyl.

The term “haloalkynyl” refers to an alkynyl group in which one or more hydrogen atoms are replaced by one or more halide atoms. In one aspect, a haloalkynyl is a C₂-C₆haloalkynyl.

The term “haloalkoxy” refers to an alkoxy group in which one or more hydrogen atoms are replaced by one or more halide atoms. In one aspect, a haloalkoxy is a C₁-C₆haloalkoxy.

The term “fluoroalkyl” refers to a alkyl in which one or more hydrogen atoms are replaced by a fluorine atom. In one aspect, a fluoroalkyl is a C₁-C₄fluoroalkyl.

The term “fluoroalkoxy” refers to an alkoxy in which one or more hydrogen atoms are replaced by a fluorine atom. In one aspect, a fluoroalkoxy is a C₁-C₄fluoroalkoxy.

The term “heteroalkyl” refers to an alkyl group in which one or more skeletal atoms of the alkyl are selected from an atom other than carbon, e.g., oxygen, nitrogen, sulfur, phosphorus or combinations thereof. In one aspect, a heteroalkyl is a C₁-C₆ heteroalkyl.

The term “heteroalkenyl” refers to an alkenyl group in which one or more skeletal atoms of the alkenyl are selected from an atom other than carbon, e.g., oxygen, nitrogen, sulfur, phosphorus or combinations thereof. In one aspect, a heteroalkenyl is a C₂-C₆ heteroalkenyl.

The term “heteroalkynyl” refers to an alkynyl group in which one or more skeletal atoms of the alkenyl are selected from an atom other than carbon, e.g., oxygen, nitrogen, sulfur, phosphorus or combinations thereof. In one aspect, a heteroalkynyl is a C₂-C₆ heteroalkynyl.

The term “bond” or “single bond” refers to a chemical bond between two atoms, or two moieties when the atoms joined by the bond are considered to be part of larger substructure.

The term “membered ring” includes any cyclic structure. The term “membered” is meant to denote the number of skeletal atoms that constitute the ring. Thus, for example, cyclohexyl, pyridinyl, pyranyl and thiopyranyl are 6-membered rings and cyclopentyl, pyrrolyl, furanyl, and thiophenyl are 5-membered rings.

The term “moiety” refers to a specific segment or functional group of a molecule. Chemical moieties are often recognized chemical entities embedded in or appended to a molecule.

A “cyano” group refers to a —CN group.

An “isocyanato” group refers to a —NCO group.

An “isothiocyanato” group refers to a —NCS group.

A “mercaptyl” group refers to a (alkyl)S— group.

A “sulfinyl” group refers to —S(═O)—.

A “sulfonyl” group refers to S(═O)₂—.

A “thiocyanato” group refers to a —CNS group.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, mercapto, alkylthio, arylthio, alkylsulfoxide, arylsulfoxide, alkylsulfone, arylsulfone, cyano, halo, carbonyl, thiocarbonyl, nitro, haloalkyl, fluoroalkyl, and amino, including mono- and di-substituted amino groups, and the protected derivatives thereof. By way of example an optional substituents may be LₛRₛ, wherein each Lₛ is independently selected from, a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, —(C₃-C₆ alkyl), or —(C₂-C₆ alkenyl); and each Rₛ is independently selected from H, alkyl, fluoroalkyl, cycloalkyl, aryl, heteroaryl, or heteroalkyl. The protecting groups that may form the protective derivatives of the above substituents may be found in sources such as Greene and Wuts, above.

In certain embodiments, the compounds presented herein possess one or more stereocenters and each center independently exists in either the R or S configuration. The compounds presented herein include all diastereomeric, enantiomeric, and epimeric forms as well as the appropriate mixtures thereof. Stereoisomers are obtained, if desired, by methods such as, the separation of stereoisomers by chiral chromatographic columns.

The methods and formulations described herein include the use of N-oxides, crystalline forms (also known as polymorphs), co-crystals, or pharmaceutically acceptable salts of compounds having the structure of Formula (I), a FLAP inhibitor or an NO modulator, as well as active metabolites of these compounds having the same type of activity. In some situations, compounds may exist as tautomers. All tautomers are included within the scope of the compounds presented herein. In addition, the compounds described herein can exist in unsolvated as well as solvated forms with pharmaceutically acceptable solvents such as water, ethanol, and the like. The solvated forms of the compounds presented herein are also considered to be disclosed herein.

### Certain Pharmaceutical Terminology

The terms “acceptable” with respect to a formulation, composition or ingredient, as used herein, means having no persistent detrimental effect on the general health of the subject being treated.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme-inhibitor, or a hormone modulator, which diminishes, or prevents the action of another molecule or the activity of a receptor site.

The term “asthma” as used herein refers to any disorder of the lungs characterized by variations in pulmonary gas flow associated with airway constriction of whatever cause (intrinsic, extrinsic, or both; allergic or non-allergic). The term asthma may be used with one or more adjectives to indicate cause.

The term “bone disease,” as used herein, refers to a disease or condition of the bone, including, but not limited to, inappropriate bone remodeling, loss or gain, osteopenia, osteomalacia, osteofibrosis, and Paget's disease [Garcia, “Leukotriene B4 stimulates osteoclastic bone resorption both in intro and in vivo”, J Bone Miner Res. 1996; 11:1619-2].

The term “cardiovascular disease,” as used herein refers to diseases affecting the heart or blood vessels or both, including but not limited to: arrhythmia; atherosclerosis and its sequalae; angina; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart or other organ or tissue; endotoxic, surgical, or traumatic shock; hypertension, valvular heart disease, heart failure, abnormal blood pressure, shock; vasoconstriction (including that associated with, migraines); vascular abnormality, inflammation, insufficiency limited to a single organ or tissue., [Lotzer K et al., “The 5-lipoxygenase pathway in arterial wall biology and atherosclerosis”, Biochim Biophys Acta 2005; 1736:30-7; Helgadottir A et al., “The gene encoding 5-lipoxygenase activating protein confers risk of myocardial infarction and stroke”, Nat. Genet. 2004 March; 36(3):233-9, Epub 2004 Feb. 8; [Heise C E, Evans J F et al., “Characterization of the human cysteinyl leukotriene 2 receptor”, J Biol Chem. 2000 Sep. 29; 275(39):30531-6].

The terra “cancer,” as used herein refers to an abnormal growth of cells which tend to proliferate in an uncontrolled way and, in some eases, to metastasize (spread). The types of cancer include, but is not limited to, solid tumors (such as those of the bladder, bowel, brain, breast, endometrium, heart, kidney, lung, lymphatic tissue (lymphoma), ovary, pancreas or other endocrine organ (thyroid), prostate, skin (melanoma) or hematological tumors (such as the leukemias) [Ding X Z et al., Anticancer Drugs. 2005 June; 16(5):467-73. Chen X et al., Clin Cancer Res. 2004 Oct. 1; 10(19):6703-9].

The term, “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered as a single agent, or as separate agents administered by the same or different route of administration or at the same or different time.

The term “dermatological disorder,” as used herein refers to a skin disorder. Such dermatological disorders include, but are not limited to, proliferative or inflammatory disorders of the skin such as, atopic dermatitis, bullous disorders, collagenosis, contact dermatitis eczema, Kawasaki Disease, rosacea, Sjogren-Larsso Syndrome, urticaria [Wedi B et al., BioDrugs. 2001; 15(11):729-43].

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing effective amount,” as used herein, refers to an amount adequate to enhance the effect of another therapeutic agent in a desired system.

The term “enzymatically cleavable linker,” as used herein refers to unstable or degradable linkages which may be degraded by one or more enzymes.

The terms “fibrosis” or “fibrosing disorder,” as used herein, refers to conditions that follow acute or chronic inflammation and are associated with the abnormal accumulation of cells and/or collagen and include but are not limited to fibrosis of individual organs or tissues such as the heart, kidney, joints, lung, or skin, and includes such disorders as idiopathic pulmonary fibrosis and cryptogenic fibrosing alveolitis [Charbeneau R P et al., “Eicosanoids: mediators and therapeutic targets in fibrotic lung disease”, Clin Sci (Lond). 2005 June; 108(6):479-91].

The term “iatrogenic” means a leukotriene-dependent or leukotriene-mediated condition, disorder, or disease created or worsened by medical or surgical therapy.

The term “inflammatory disorders” refers to those diseases or conditions that are characterized by one or more of the signs of pain (dolor, from the generation of noxious substances and the stimulation of nerves), heat (color, from vasodilatation), redness (rubor, from vasodilatation and increased blood flow), swelling (tumor, from excessive inflow or restricted outflow of fluid), and loss of function (functio laesa, which, may be partial or complete, temporary or permanent). Inflammation takes many forms and includes, but is not limited to, inflammation that is one or more of the following: acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative. Inflammatory disorders further include, without being limited to those affecting the blood vessels (polyarteritis, temporal arteritis); joints (arthritis: crystalline, osteo-, psoriatic, reactive, rheumatoid, Reiter's); gastrointestinal tract (Disease,); skin (dermatitis); or multiple organs and tissues (systemic lupus erythematosus) [Harrison's Principles of Internal Medicine, 16^{th} Edition, Kasper D L, et al, Editors; McGraw-Hill, publishers].

The term “interstitial cystitis” refers to a disorder characterized by lower abdominal discomfort, frequent and sometimes painful urination that is not caused by anatomical abnormalities, infection, toxins, trauma or tumors [Bouchelouche K et ah, “The cysteinyl leukotriene D4 receptor antagonist montelukast for the treatment of interstitial cystitis”, J Urol 2001; 166:1734].

The term “leukotriene-driven mediators,” as used herein, refers to molecules able to be produced in a patient that may result from excessive production of leukotriene stimulation of cells, such as, by way of example only, LTB₄, LTC₄, LTE₄, cysteinyl leukotrienes, monocyte inflammatory protein (MIP-1α), interleukin-8 (IL-8), interleukin-4 (IL-4), interleukin-13 (IL-13), monocyte chemoattractant protein (MCP-1), soluble intracellular adhesion molecule (sICAM; soluble ICAM), myeloperoxidase (MPO), eosinophil peroxidase (EPO), and general inflammation molecules such as interleukin-6 (IL-6), C-reactive protein (CRP), and serum amyloid A protein (SAA).

The term “leukotriene-dependent”, as used herein, refers to conditions or disorders that would not occur, or would not occur to the same extent, in the absence of one or more leukotrienes.

The term “leukotriene-mediated”, as used herein, refers to refers to conditions or disorders that might occur in the absence of leukotrienes but can occur in the presence of one or more leukotrienes.

The term “leukotriene-responsive patient” refers to a patient who has been selected by either specific genotyping of FLAP haplotype, or genotyping of one or more other genes in the leukotriene pathway and, or, by specific phenotyping of patients either by previous positive clinical response to another leukotriene modulator such as zileuton (Zyflo™), montelukast (Singulair™), pranlukast (Onon™), zafirlukast (Accolate™), or by their profile of leukotriene-driven mediators that indicate excessive leukotriene stimulation of inflammatory cells, as most likely to respond favorably to leukotriene modulator therapy. In the case of the present patent application, polymorphisms in any of the synthetic or signaling genes dedicated to the leukotriene pathway could result in a patient who is more responsive or less responsive to leukotriene modulator therapy (either enzyme inhibitor or receptor antagonists). The genes dedicated to the leukotriene pathway are 5-lipoxygenase (ALOX5), 5-lipoxygenase-activating protein (ALOX5AP), LTA₄ hydrolase (LTA₄H), LTC₄ synthase (LTC₄S), LTB₄ receptor 1 (LTB₄R1; BLT1), LTB₄ receptor 2 (LTB₄R2; BLT2), cysteinyl leukotriene receptor 1 (CysLT₁R), cysteinyl leukotriene receptor 2 (CysLT₂R). Any polymorphisms in any gene or combination of polymorphisms may result in altered sensitivity of the patient to therapy aimed at reducing the pathological effects of leukotrienes. Selection of patients who might best respond to leukotriene modulator therapies may include knowledge of polymorphisms in the leukotriene pathway genes and also knowledge of the expression of leukotriene-driven mediators. Patient selection could be made on the basis of leukotriene pathway genotype alone, phenotype alone (biomarkers) or a combination of both genotype and phenotype.

The terms “kit” and “article of manufacture” are used as synonyms.

The term “LTNO” refers to any agent can is a FLAP inhibitor and an NO modulator, or the combination of a FLAP inhibitor agent and an NO modulator agent.

A “metabolite” of a compound disclosed herein is a derivative of that compound that is formed when the compound is metabolized. The terra “active metabolite” refers to a biologically active derivative of a compound, that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996), Metabolites of the compounds disclosed herein can be identified either by administration of compounds to a host and analysis of tissue samples from the host, or by incubation of compounds with hepatic cells in vitro and analysis of the resulting compounds.

The term “modulate,” as used herein, means to directly or indirectly interact with a target so as to alter the activity of the target, including, by way of example only, to enhance the activity of the target, to maintain the activity of the target, to extend the activity of the target, to alter the bioavailability of the target, to decrease the activity of the target, to poison the activity of the target, to destroy the target or to increase the clearance of the target.

The term “modulator,” as used herein, refers to a molecule or agent that modulates a target. In one aspect, a modulator is an antagonist. In another embodiment, a modulator is an inhibitor.

The term “NO modulator,” as used herein, refers to a molecule or moiety that acts as a source of NO, can provide NO directly or indirectly, increase levels of NO directly or indirectly, or maintain levels of NO directly or indirectly. Direct refers to an interaction whose immediate effect is to produce a desired result (e.g., an agent that releases NO). An indirect action encompasses all other interactions. An agent that provides NO directly is also referred to as a “direct NO provider”; an agent that modulates levels of NO directly is also referred to as a “direct NO level modulator”; an agent that maintains levels of NO directly is also referred to as a “direct NO maintainer.”

The terms “neurogenerative disease” or “nervous system disorder,” as used herein, refers to conditions that alter the structure or function of the brain, spinal cord or peripheral nervous system, including but not limited to Alzheimer's Disease, cerebral edema, cerebral ischemia, multiple sclerosis, neuropathies. Parkinson's Disease, those found after blunt or surgical trauma (including post-surgical cognitive dysfunction and spinal cord or brain stem injury), as well as the neurological aspects of disorders such as degenerative disk disease and sciatica. The acronym “CNS” refers to disorders of the central nervous system, i.e., brain and spinal cord [Sugaya K, et al., Jpn J Pharmacol. 2000 February; 82(2):85-94; Yu G L, et al., Pharmacology. 2005 January; 73(1):31-40; [Zhang W P, et al., Acta Pharmacol Sin. 2002 October; 23(10):871-7].

The terms “ocular disease” or “ophthalmic disease,” as used herein, refer to diseases which affect the eye or eyes and potentially the surrounding tissues as well. Ocular or ophthalmic diseases include, but are not limited to, conjunctivitis, retinitis, scleritis, uveitis, allergic conjunctivitis, vernal conjunctivitis, papillary conjunctivitis [Toriyama S., Nippon Ganka Gakkai Zasshi. 2000 June; 104(6):396-40; [Chen F, et al., Ophthalmic Res. 1991; 23(2):84-91].

By “pharmaceutically acceptable,” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a formulation of a compound that does not cause significant irritation to an organism to which it is administered and does not abrogate the biological activity and properties of the compound. In some embodiments, pharmaceutically acceptable salts are obtained by reacting a compound of Formula (I), a FLAP inhibitor, or an NO modulator with acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutically acceptable Salts may also be obtained by reacting a compound described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with, amino, acids such as arginine, lysine, and the like, or by other methods known in the art

The term, “pharmaceutical combination,” as used herein, means a product that results from the mixing or combining of more than one active ingredient and includes both fixed and non-fixed combinations of the active ingredients. The term “fixed combination” means that the active ingredients, e.g. a NO modulator (including agents that release NO in a patient or agents that otherwise raise NO to a patient or, alternatively, maintain production of NO or agents that otherwise maintain levels of NO in a patient) and a FLAP inhibitor, are both administered to a patient simultaneously in the form of a single entity or dosage. The term “non-fixed combination” means that the active ingredients, e.g., a NO modulator (including agents that release NO in a patient or agents that otherwise raise NO in a patient or, alternatively, maintain production of NO or agents that otherwise maintain levels of NO in a patient) and a FLAP inhibitor are administered to a patient as separate entities either simultaneously, concurrently, or sequentially with no specific intervening time limits, wherein such administration provides effective levels of the two compounds in the body of the patient. The latter also applies to cocktail therapy, e.g., the administration of three or more active ingredients.

The term “pharmaceutical composition” refers to a mixture of one or more NO modulators (including agents that release NO in a patient or agents that otherwise raise NO in a patient or, alternatively, maintain production of NO or agents that otherwise maintain levels of NO in a patient) and one or more FLAP inhibitors with other chemical components, such as carriers, stabilizers, diluents, dispersing agents, suspending agents, thickening agents, and/or excipients. The pharmaceutical composition facilitates administration of the combination therapy to a patient. Multiple techniques of administering a combination therapy exist in the art including, but not limited to: intravenous, oral, aerosol, rectal, vaginal, urethral, buccal, parenteral, ophthalmic, pulmonary and topical administration.

A “prodrug” refers to an agent that is converted into the parent drug in vivo. Prodrugs are often useful because, in some situations, they may be easier to administer than the parent drug, in some embodiments, prodrugs are bioavailable by oral administration whereas the parent is not. In other embodiments, the prodrug has improved solubility in pharmaceutical compositions over the parent drag. An example, without limitation, of a prodrug is a compound which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug is a short peptide (polyaminoacid) bonded to art acid group where the peptide is metabolized to reveal the active moiety.

The term “respiratory disease,” as used herein, refers to diseases affecting the organs that are involved in breathing, such as the nose, throat, larynx, trachea, bronchi, and lungs. Respiratory diseases include, but are not limited to, asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia [Evans J F, Allergology International 2005; 54:187-90); Kemp J P., IDrugs. 2000 April; 3(4):430-41; Riccioni G, et al., Allergy Asthma Proc. 2004 November-December; 25(6):445-8].

The term “subject” or “patient” encompasses mammals and non-mammals and can refer to an individual afflicted with or prone to a condition, disease or disorder as specified herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs and cats; laboratory animals including rodents, such as rats, mice and guinea pigs, and the like. Examples of non-mammals include, but are not limited to, birds, fish and the like. In one embodiment of the methods and compositions provided herein, the mammal is a human.

lire terms “treat”, “treating,” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

### Routes of Administration

Suitable routes of administration include, for example, oral, rectal, transmucosal, transdermal, pulmonary, ophthalmic or intestinal administration; parenteral delivery, including intramuscular, subcutaneous, intravenous, intramedullary injections, as well as intrathecal direct intraventricular, intraperitoneal, or intranasal injections.

In some embodiments, compound(s) described herein are administered in a local rather than systemic manner, for example, via injection of the compound(s) directly into an organ, often in a depot preparation or sustained release formulation. In one aspect, long acting formulations are administered by implantation (for example subcutaneously or intramuscularly) or by intramuscular injection. In another aspect, administration of the drug is achieved in a targeted drug delivery system, for example, in a liposome coated with organ-specific antibody. The liposomes will be targeted to and taken up selectively by the organ. In some embodiments, the drug is provided in the form of a rapid release formulation, in the form of an extended release formulation, or in the form of an intermediate release formulation.

### Pharmaceutical Composition/Formulation

Pharmaceutical compositions are formulated in a conventional manner using one or more physiologically acceptable carriers comprising excipients and auxiliaries which facilitate processing of the active compounds into preparations which are then used pharmaceutically. Proper formulation is dependent upon the route of administration chosen. Any of the well-known techniques, carriers, and excipients are suitable and as understood in the art. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co. Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Provided herein are pharmaceutical compositions comprising a compound of Formula (I), a FLAP inhibitor, an NO modulator, or a mixture of a FLAP inhibitor and an NO modulator, and a pharmaceutically acceptable diluent(s), excipient(s), or carrier(s). In some embodiments, the compounds described herein are administered as pharmaceutical compositions in which compounds of Formula (I), a FLAP inhibitor, an NO modulator, or a mixture of a FLAP inhibitor and an NO modulator, are mixed with other active ingredients, as in a further combination therapy.

A pharmaceutical composition, as used herein, refers to a mixture of a compound of Formula (I), a FLAP inhibitor, an NO modulator, or a mixture of a FLAP inhibitor and an NO modulator, with other chemical components, such as carriers, stabilizers, diluents, dispersing agents, suspending agents, thickening agents, and/or excipients. The pharmaceutical composition facilitates administration of the compound to an organism. In practicing the methods of treatment or use provided herein, therapeutically effective amounts of compounds of Formula (I), a FLAP inhibitor, an NO modulator, or a mixture of a FLAP inhibitor and an NO modulator, provided herein are administered in a pharmaceutical composition to a mammal having a disease or condition to be treated. Preferably, the mammal is a human. A therapeutically effective amount can vary widely depending on the severity of the disease, the age and relative health of the subject, the potency of the compound used and other factors. The compounds are used singly or in combination with one or more therapeutic agents as components of mixtures.

For intravenous injections, compounds of Formula (I), a FLAP inhibitor, an NO modulator, or a mixture of a FLAP inhibitor and an NO modulator, are formulated in aqueous solutions, preferably in physiologically compatible buffers such as Hank's solution. Ringer's solution, or physiological saline buffer. For transmucosal administration, penetrants appropriate to the battier to be permeated are used in the formulation. Such penetrants are generally known in the art. For other parenteral injections, appropriate formulations include aqueous or nonaqueous solutions, preferably with physiologically compatible buffers or excipients. Such excipients are generally known in the art.

For oral administration, compounds of Formula (I), a FLAP inhibitor, an NO modulator, or a mixture of a FLAP inhibitor and an NO modulator, are formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, tillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate.

Dragee cores are provided with suitable coatings. For this purpose, concentrated sugar solutions are used, which optionally contain gum arable, talc, polyvinylpyrrolidone, carbopol gel, polyethylene glycol, and/or titanium dioxide, lacquer solutions, and suitable organic solvents or solvent mixtures. In some embodiments, dyestuffs or pigments are added to the tablets or dragee coatings for identification or to characterize different combinations of active compound doses.

Pharmaceutical preparations for oral administration include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added. All formulations for oral administration should be in dosages suitable for such administration.

For buccal or sublingual administration, the compositions are in the form of tablets, lozenges, or gels formulated in a conventional manner. Parental injections involve bolus injection or continuous infusion. Formulations for injection are presented in unit dosage form, e.g., in ampoules or in multi-dose containers, with an added preservative. In one aspect, the pharmaceutical composition of Formula (I), a FLAP inhibitor, an NO modulator, or a mixture of a FLAP inhibitor and an NO modulator, are in a form suitable for parenteral injection as a sterile suspensions, solutions or emulsions in oily or aqueous vehicles, and optionally contain formulatory agents such as suspending, stabilizing and/or dispersing agents. Pharmaceutical formulations for parenteral administration include aqueous solutions of the active compounds in water-soluble form. Additionally, suspensions of the active compounds are prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions contain substances which increase the viscosity of the suspension, such as sodium carboxymethyl cellulose, sorbitol or dextran. Optionally, the suspension also contain suitable stabilizers or agents which increase the solubility of the compounds to allow for the preparation of highly concentrated solutions. Alternatively, the active ingredient is in powder form for constitution with a suitable vehicle, e.g., sterile pyrogen-free water, before use.

In some embodiments, the compounds of Formula (I), a FLAP inhibitor, an MO modulator, or a mixture of a FLAP inhibitor and an NO modulator, are administered topically and are formulated into a variety of topically administrable compositions, such as solutions, suspensions, lotions, gels, pastes, medicated sticks, balms, creams or ointments. In some embodiments, such pharmaceutical compounds contain solubilizers, stabilizers, tonicity enhancing agents, buffers and preservatives.

In some embodiments, formulations suitable for transdermal administration of compounds having the structure of Formula (I), a FLAP inhibitor, an NO modulator, or a mixture of a FLAP inhibitor and an NO modulator, employ transdermal delivery devices and transdermal delivery patches and in some embodiments are lipophilic emulsions or buffered, aqueous solutions, dissolved and/or dispersed in a polymer or an adhesive. In some embodiments, such patches are constructed for continuous, pulsatile, or on demand delivery of pharmaceutical agents. In yet other embodiments, transdermal delivery of the compounds of Formula (I), a FLAP inhibitor, an NO modulator, or a mixture of a FLAP inhibitor and an NO modulator, is accomplished by means of iontophoretic patches and the like. In one aspect, transdermal patches provide controlled delivery of the compounds Formula (I), a FLAP inhibitor, an NO modulator, or a mixture of a FLAP inhibitor and an NO modulator. In some embodiments, the rate of absorption is slowed by using rate-controlling membranes or by trapping the compound within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation, the compounds of Formula (I), a FLAP inhibitor, an NO modulator, or a mixture of a FLAP inhibitor and an NO modulator, is in a form as an aerosol, a mist or a powder. Pharmaceutical compositions of Formula (I), a FLAP inhibitor, an NO modulator, or a mixture of a FLAP inhibitor and an NO modulator, are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit is determined by providing a valve to deliver a metered amount. Capsules and cartridges of, such as, by way of example only, gelatin for use in an inhaler or insufflator may be formulated containing a powder mix of the compound and a suitable powder base such as lactose or starch.

In one aspect, the compounds of Formula (I), a FLAP inhibitor, an NO modulator, or a mixture of a FLAP inhibitor and an NO modulator, are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Formulations suitable for vaginal administration are presented as pessaries, tablets, tampons, creams, gels, pastes, foams or spray formulations containing in addition to the active ingredient(s) such excipients as are known in the art to be appropriate. Further suitable formulations include urethral suppositories, such as bougies.

Pharmaceutical compositions are formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients are used as suitable and as understood in the art. In one aspect, pharmaceutical compositions comprising a compound of Formula (I), a FLAP inhibitor, an NO modulator, or a mixture of a FLAP inhibitor and an NO modulator, are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient (i.e. inactive ingredients) and a compound of Formula (I), a FLAP inhibitor, an NO modulator, or a mixture of a FLAP inhibitor and an NO modulator, described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, compounds exist as tautomers. All tautomers are included within the scope of the compounds presented herein. Additionally, the compounds described herein exist in unsolvated as well as solvated forms with pharmaceutically acceptable solvents such, as water, ethanol, and the like. The solvated forms of the compounds presented herein are also considered to be disclosed herein. In addition, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In some situations, the pharmaceutical compositions also contain other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A composition comprising a compound of Formula (I), a FLAP inhibitor, an NO modulator, or a mixture of a FLAP inhibitor and an NO modulator, illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition may include a gel formulation. In other embodiments, fee liquid composition is aqueous.

In some situations, aqueous suspension also contain one or more polymers as suspending agents. Useful polymers include water-soluble polymers such as cellulosic polymers, e.g., hydroxypropyl methylcellulose, and water-insoluble polymers such as cross-linked carboxyl-containing polymers. In some situations, compositions also comprise an mucoadhesive polymer, selected for example from carboxymethylcellulose, carbomer (acrylic acid polymer), polymethylmethacrylate), polyacrylamide, polycarbophil, acrylic acid/butyl acrylate copolymer, sodium alginate and dextran.

In one aspect, pharmaceutical compositions also include solubilizing agents to aid in the solubility of a compound of Formula (I), a FLAP inhibitor, an NO modulator, or a mixture of a FLAP inhibitor and an NO modulator. The term “solubilizing agent” generally includes agents that result in formation of a micellar solution or a true solution of the agent. Certain acceptable nonionic surfactants, for example polysorbate 80, can be useful as solubilizing agents, as can ophthalmically acceptable glycols, polyglycols, e.g., polyethylene glycol 400, and glycol ethers.

In another aspect, pharmaceutical compositions also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, pharmaceutical compositions also include one or more salts in an amount required to bring osmolality of the composition into art acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful compositions may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In other embodiments, pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In other embodiments, pharmaceutical compositions include one or more antioxidants to enhance chemical stability where required. Suitable antioxidants include, by way of example only, ascorbic acid and sodium metabisulfite.

In some embodiments, aqueous suspension compositions are packaged in single-dose non-reclosable containers. Alternatively, multiple-dose reclosable containers are used, in which case it is typical to include a preservative in the composition.

Alternatively, other delivery systems for hydrophobic pharmaceutical compounds are employed. Liposomes and emulsions are well known examples of delivery vehicles or carriers for hydrophobic drugs. Certain organic solvents such as N-methylpyrrolidone also are employed, although usually at the cost of greater toxicity. In some embodiments, the compounds are delivered using a sustained-release system, such as semipermeable matrices of solid hydrophobic polymers containing the therapeutic agent. Various sustained-release materials have been established and are well known by those skilled in the art. Sustained-release capsules, depending on their chemical nature, release she compounds for a few hours up to over 10 hours. Depending on the chemical nature and the biological stability of the therapeutic reagent, additional strategies for protein stabilization are employed.

In one aspect, the formulations described herein benefit from, antioxidants, metal chelating agents, thiol containing compounds and other general, stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 3.0 mM EDTA, (e) about 0.01% to about 2% w/v ascorbic acid, (f) 0.003% to about 0.02% w/v polysorbate 80, (g) 0.001% to about 0.05% w/v polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Methods of Dosing and Treatment Regimens

For convenience, the administration methods of dosing and treatment regimens described in this section and other parts herein use a single formula, such as “Formula (I),” by way of example. In addition, the administration methods and treatment methods described herein apply equally well to all formulas, or combinations or mixtures of formulas or agents, presented herein that fail within the scope of Formula (I), FLAP inhibitors, NO modulators, as well as to all of the specific compounds that fall within the scope of these general categories.

In one embodiment, the compounds of Formula (I) are used in the preparation of medicaments for the treatment of leukotriene-dependent or leukotriene mediated diseases or conditions. In addition, a method, for treating any of the diseases or conditions described herein in a subject in need of such treatment, involves administration of pharmaceutical compositions containing at least one compound of Formula (I), or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject

The compositions containing the compound(s) described herein are administered for prophylactic and/or therapeutic treatments. In therapeutic applications, the compositions are administered to a patient already suffering from a disease or condition, in an amount sufficient to cure or at least partially arrest the symptoms, of the disease or condition. Amounts effective for this use will depend on the severity and course of the disease or condition, previous therapy, the patient's health status, weight, and response to the drugs, and the judgment of the treating physician. It is considered well within the skill of the art for one to determine such therapeutically effective amounts by routine experimentation (including, but not limited to, a dose escalation clinical trial).

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. It is considered well within the skill of the art for one to determine such prophylactically effective amounts by routine experimentation (e.g., a dose escalation clinical trial). When used in a patient, effective amounts for this use will depend on the severity and course of the disease, disorder or condition, previous therapy, the patient's health status and response to the drugs, and the judgment of the treating physician.

In one aspect in the case wherein she patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the compounds are given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained, in one aspect, patients require intermittent treatment on a long-term basis upon any recurrence of symptoms.

The amount, of a given agent that will correspond to such an amount will vary depending upon factors such as the particular compound, disease condition and its severity, the Identity (e.g., weight) of the subject or host in need of treatment, but can nevertheless be routinely determined in a manner known in the art according to the particular circumstances surrounding the case, including, e.g., the specific agent being administered, the route of administration, the condition being treated, and the subject or host being treated. In general, however, doses employed for adult human treatment will typically be in the range of 0.02-5000 mg per day. In one aspect, doses employed for adult human treatment will be in the range of 1-1500 mg per day. In some embodiments, the desired, dose is conveniently presented in a single dose or as divided doses administered simultaneously (or over a short period of time) or at appropriate intervals, for example as two, three, four or more sub-doses per day.

In some embodiments, the pharmaceutical compositions described herein are in unit dosage forms suitable for single administration of precise dosages. In unit dosage form, the formulation is divided into unit doses containing appropriate quantities of one or more compound. In one aspect, the unit dosage is in the form of a package containing discrete quantities of the formulation. Non-limiting examples are packaged tablets or capsules, and powders in vials or ampoules, in one aspect, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi-dose containers, with an added preservative.

The daily dosages appropriate for the compounds of Formula (I) described herein are from about 0.01 to 2.5 mg/kg per body weight. An indicated daily dosage in the larger mammal, including, but not limited to, humans, is in the range from about 0.5 mg to about 1000 mg, conveniently administered in divided doses, including, but not limited to, up to four times a day or in extended release form. Suitable unit dosage forms for oral administration comprise from about 5 to 1000 mg active ingredient. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it cart be expressed as the ratio between LD₅₀ and ED₅₀. Compounds exhibiting high therapeutic indices are preferred. The data obtained from cell culture assays and animal studies is used in formulating a range of dosage for use in human. The dosage of such compounds lies preferably within a range of circulating concentrations that include the ED₅₀ with minimal toxicity. In one aspect, the dosage varies within this range depending upon the dosage form employed and the route of administration utilized.

Use of Combination Compounds and Methods to Prevent and/or Treat NO-Dependent, NO-Mediated, Leukotriene-Dependent or Leukotriene Mediated Diseases or Conditions

For convenience, the use of the combination compounds and methods to prevent and/or treat NO-dependent, NO-mediated (or NO-related), leukotriene-dependent or leukotriene mediated (or leukotriene-related) diseases or conditions described in this section and other parts herein use a single formula, such as “Formula (I),” by way of example. In addition, the administration methods and treatment methods described herein apply equally well to all formulas, or combinations or mixtures of formulas or agents, presented herein that fall within the scope of Formula (I), FLAP inhibitors, NO modulators, as well as to all of the specific compounds that fall within the scope of these general categories.

The combination therapies of NO-dependent, NO-mediated (or NO-related), leukotriene-dependent or leukotriene mediated (or leukotriene-related) diseases or conditions is designed to modulate the level of NO and modulate the activity of FLAP. Such NO modulation is as described herein. Such FLAP modulation includes, by way of example only, inhibiting or antagonizing FLAP activity. For example, a FLAP inhibitor can be administered in order to decrease synthesis of leukotrienes within the individual, or possibly to downregulate or decrease the expression or availability of the FLAP mRNA or specific splicing variants of the FLAP mRNA. Downregulation or decreasing expression or availability of a native FLAP mRNA or of a particular splicing variant could minimize the expression or activity of a defective nucleic acid or the particular splicing variant and thereby minimize the impact of the defective nucleic acid or the particular splicing variant.

In accordance with one aspect, compositions and methods described herein include compositions and methods for treating, preventing, reversing, halting or slowing the progression of NO-dependent, NO-mediated (or NO-related), leukotriene-dependent or leukotriene mediated (or leukotriene-related) diseases or conditions once it becomes clinically evident, or treating the symptoms associated with or related to NO-dependent, NO-mediated (or NO-related), leukotriene-dependent or leukotriene mediated (or leukotriene-related) diseases or conditions, by administering to the subject a compound of Formula (I) or pharmaceutical composition or medicament which includes a compound of Formula (I). The subject may already have a NO-dependent, NO-mediated (or NO-related), leukotriene-dependent or leukotriene mediated (or leukotriene-related) disease or condition at the time of administration, or be at risk of developing a NO-dependent, NO-mediated (or NO-related), leukotriene-dependent or leukotriene mediated (or leukotriene-related) disease or condition. The symptoms of NO-dependent, NO-mediated (or NO-related), leukotriene-dependent or leukotriene mediated (or leukotriene-related) diseases or conditions in a subject is determined by one skilled in the art and are described in standard textbooks.

The activity of 5-lipoxygenase activating protein in a mammal is directly or indirectly modulated by the administration of (at least once) an effective amount of at least one compound of Formula (I), or pharmaceutical composition or medicament which includes a compound of Formula (I), to a mammal. Such modulation includes, but is not limited to, reducing and/or inhibiting the activity of 5-lipoxygenase activating protein. In addition, the activity of leukotrienes in a mammal is directly or indirectly modulated, including reducing and/or inhibiting, by the administration of (at least once) an effective amount of at least one compound of Formula (I), or pharmaceutical composition or medicament which includes a compound of Formula (I), to a mammal. Such modulation includes, but is not limited to, reducing and/or inhibiting the activity of 5-lipoxygenase activating protein.

In one aspect, prevention and/or treatment of NO-dependent, NO-mediated (or NO-related), leukotriene-dependent or leukotriene mediated (or leukotriene-related) mediated diseases or conditions comprises administering to a mammal at least once an effective amount of at least one compound of Formula (I), or pharmaceutical composition or medicament which includes a compound of Formula (I). By way of example, the prevention and/or treatment of inflammation diseases or conditions comprises administering to a mammal at least once an effective amount of at least one compound of Formula (I), or pharmaceutical composition or medicament which includes a compound of Formula (I). NO-dependent, NO-mediated (or NO-related), leukotriene-dependent or leukotriene mediated (or leukotriene-related) diseases or conditions that may be treated by a method comprising administering to a mammal at least once an effective amount of at least one compound of Formula (I), or pharmaceutical composition or medicament which includes a compound of Formula (I), include, but are not limited to, bone diseases and disorder, cardiovascular diseases and disorders, inflammatory diseases and disorders, dermatological diseases and disorders, ocular diseases and disorders, cancer and other proliferative diseases and disorders, respiratory diseases and disorder, and non-cancerous disorders.

By way of example only, included in the prevention/treatment methods described herein are methods for treating respiratory diseases comprising administering to the mammal at least once an effective amount of at least one compound of Formula (I), or pharmaceutical composition or medicament which includes a compound of Formula (I). By way of example the respiratory disease may be asthma; see Riccioni et al. Ann. Clin. Lab. Sci., v34, 379-387 (2004). In addition, the respiratory disease may include, but is not limited to, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, allergic rhinitis, vascular responses, endotoxin shock, fibrogenesis, pulmonary fibrosis, allergic diseases, chronic inflammation, and adult respiratory distress syndrome.

By way of example only, included in such treatment methods are methods for preventing chronic obstructive pulmonary disease comprising administering to the mammal at least once an effective amount of at least one compound of formula (I), or pharmaceutical composition or medicament which includes a compound of Formula (I). In addition, chronic obstructive pulmonary disease includes, but is not limited to, chronic bronchitis or emphysema, pulmonary hypertension, interstitial lung fibrosis and/or airway inflammation and cystic fibrosis.

By way of example only, included in such treatment methods are methods for preventing increased mucosal secretion and/or edema in a disease or condition comprising administering to the mammal at least once an effective amount of at least one compound of Formula (I), or pharmaceutical composition or medicament which includes a compound of Formula (I).

By way of example only, included in the prevention/treatment methods described herein are methods for preventing or treating vasoconstriction, atherosclerosis and its sequalae myocardial ischemia, myocardial infarction, aortic aneurysm, vasculitis and stroke comprising administering at least once to the mammal art effective amount of at least one compound of Formula (I), or pharmaceutical composition or medicament which includes a compound of Formula (I); see Jala et al. Trends in Immunol., v25, 315-322 (2004) and mehrabian et al, Curr. Opin. Lipidol., v14, 447-457 (2003).

By way of example only, included in the prevention/treatment methods described herein are methods for reducing cardiac reperfusion injury following myocardial ischemia and/or endotoxic shock comprising administering at least once to the mammal an effective amount of at least one compound of Formula (I), or pharmaceutical composition or medicament which includes a compound of Formula (I).

By way of example only, included in the prevention/treatment methods described herein are methods for reducing the constriction of blood vessels in a mammal comprising administering at least once to the mammal an effective amount of at least one compound of Formula (I), or pharmaceutical composition or medicament which includes a compound of Formula (I).

By way of example only, included in the prevention/treatment methods described herein are methods for lowering or preventing an increase in blood pressure of a mammal comprising administering at least once to the mammal an effective amount of at least one compound of Formula (I), or pharmaceutical composition or medicament which includes a compound of Formula (I).

By way of example only, included in the prevention/treatment methods described herein are methods for preventing eosinophil and/or basophil and/or dendritic cell and/or neutrophil and/or monocyte recruitment comprising administering at least once to the mammal an effective amount of at least one compound of Formula (I), or pharmaceutical composition or medicament which includes a compound of Formula (I).

By way of example only, included in the prevention/treatment methods described herein are methods for the prevention or treatment of abnormal bone remodeling, loss or gain, including diseases or conditions as, by way of example, osteopenia, osteoporosis, Paget's disease, cancer and other diseases comprising administering at least once to the mammal an effective amount of at least one compound of Formula (I), or pharmaceutical composition or medicament which includes a compound of Formula (I).

By way of example only, included is the prevention treatment methods described herein are methods for preventing ocular inflammation and allergic conjunctivitis, vernal keratoconjunctivitis, and papillary conjunctivitis comprising administering at least once to the mammal an effective amount of at least one compound of Formula (I), or pharmaceutical composition or medicament which includes a compound of Formula (I): see Lambiase et al, Arch. Opthalmol., v121, 615-620 (2003).

By way of example only, included in the prevention/treatment methods described herein are methods for preventing CNS disorders comprising administering at least once to the mammal an effective amount of at least one compound of Formula (I), or pharmaceutical composition or medicament which includes a compound of Formula (I), CNS disorders include, but are not limited to, multiple sclerosis, Parkinson's disease, Alzheimer's disease, stroke, cerebral ischemia, retinal ischemia, postsurgical cognitive dysfunction, migraine, peripheral neuropathy/neuropathic pain, spinal cord injury, cerebral edema and head injury.

By way of example only, included in the prevention/treatment methods described herein are methods for the treatment of cancer comprising administering at least once to the mammal an effective amount of at least one compound of Formula (I), or pharmaceutical composition or medicament which includes a compound of Formula (I). The type of cancer may include, but is not limited to, pancreatic cancer and other solid or hematological tumors, see Poff and Balazy, Curr. Drug Targets Inflamm. Allergy, v3, 19-33 (2004) and Steele et al, Cancer Epidemiology & Prevention, v8, 467-483 (1999).

By way of example only, included in the prevention/treatment methods described herein are methods for preventing endotoxic shock and septic shock comprising administering at least once to the mammal an effective amount of at least one compound of Formula (I), or pharmaceutical composition or medicament which includes a compound of Formula (I).

By way of example only. Included in the prevention/treatment methods described herein methods for preventing rheumatoid arthritis and osteoarthritis comprising administering at least once to the mammal an effective amount of at least one compound of Formula (I), or pharmaceutical composition or medicament which includes a compound of Formula (I).

By way of example only, included in the prevention/treatment methods described herein are methods for preventing increased GI diseases comprising administering at least once to the mammal an effective amount of at least one compound of Formula (I), or pharmaceutical composition or medicament which includes a compound of Formula (I). Such GI diseases include, by way of example only, inflammatory bowel disease (IBD), colitis and Crohn's disease.

By way of example only, included in the prevention/treatment methods described herein are methods for the reduction of inflammation while also preventing transplant rejection or preventing or treating tumors or accelerating the healing of wounds comprising administering at least once to the mammal an effective amount of at least one compound of Formula (I), or pharmaceutical composition or medicament which includes a compound of Formula (I).

By way of example only, included in the prevention/treatment methods described herein are methods for the prevention or treatment of rejection or dysfunction in a transplanted organ or tissue comprising administering at least once to the mammal an effective amount of at least one compound of Formula (I), or pharmaceutical composition or medicament which includes a compound of Formula (I).

By way of example only, included in the prevention/treatment methods described herein are methods for treating type II diabetes comprising administering to at least once to the mammal an effective amount of at least one compound of Formula (I), or pharmaceutical composition or medicament which includes a compound of Formula (I).

By way of example only, included in the prevention/treatment methods described herein are methods for treating inflammatory responses of the skin comprising administering at least once to the mammal an effective amount of at least one compound of Formula (I), or pharmaceutical composition or medicament which includes a compound of Formula (I). Such inflammatory responses of the skin include, by way of example, psoriasis, dermatitis, contact dermatitis, eczema, urticaria, rosacea, wound healing and scarring. In another aspect are methods for reducing psoriatic lesions in the skin, joints, or other tissues or organs, comprising administering at least once to the mammal an effective amount of at least one compound of Formula (I), or pharmaceutical composition or medicament which includes a compound of Formula (I).

By way of example only, included in the prevention/treatment methods described herein are methods for the treatment of cystitis, including, by way of example only, interstitial cystitis, comprising administering at least once to the mammal an effective amount of at least one compound of Formula (I), or pharmaceutical composition or medicament which includes a compound of Formula (I).

By way of example only, included in the prevention/treatment methods described herein are methods for the treatment of metabolic syndromes such as Familial Mediterranean Fever comprising administering at least, once to the mammal an effective amount of at least one compound of Formula (I), or pharmaceutical composition or medicament which includes a compound of Formula (I).

The present disclosure encompasses combination compounds, compositions, and methods of treating and preventing conditions and disorders which include, but are not limited to, hypertension, atherosclerosis, diabetes, chronic heart failure, aortic and cerebral aneurysms, stroke, sepsis, osteoporosis and bone resorption disorders, autoimmune diseases, including lupus erythematosus and multiple sclerosis, arthritis including rheumatoid arthritis and osteoarthritis, neurological diseases including Alzheimer's dementia, amyotrophic lateral sclerosis and Parkinson's disease, inflammatory diseases (e.g., sepsis, adult respiratory distress syndrome, inflammatory bowel disease, and disseminated intravascular coagulation), fibromyalgia, pain resulting from inflammation, including hyperalgesia and allodynia, neuropathic pain, and cancer including colon and pancreatic cancer).

The present disclosure also encompasses combination compounds, compositions, and methods of treating and preventing respiratory conditions which include, but are not limited to: apnea, asbestosis, atelectasis, berylliosis, bronchial diseases, bronchiectasis, bronchiolitis, bronchiolitis obliterans organizing pneumonia, bronchitis, bronchopulmonary dysplasia, common, cold, cough, empyema—pleural, epiglottis, hemoptysis, hypoxia, kartagener syndrome, meconium aspiration, pleural disease, pleural effusion, pleurisy, pneumonia pneumothorax, pulmonary alveolar proteinosis, chronic obstructive pulmonary disease, pulmonary hypertension, pulmonary edema, pulmonary embolism, pulmonary emphysema, pulmonary fibrosis, respiratory distress syndrome—newborn, respiratory hypersensitivity, respiratory tract diseases, respiratory tract infections, rhinoscleroma, scimitar syndrome, severe acute respiratory syndrome, silicosis, stridor, tracheal stenosis, Wegener's granulomatosis, occupational lung disease and whooping cough in patients, and in humans in particular.

The present disclosure also encompasses combination compounds, compositions, and methods of treating and preventing chronic and/or acute diseases and conditions. The present disclosure also encompasses combination compounds, compositions, and methods of treating and preventing local and/or systemic respiratory and/or cardiovascular conditions. For example, they may be used to treat or prevent inflammation of a wide variety of tissues and organs such as, but are not limited to, the skin, muscles, connective tissue (e.g., tendons and ligaments), blood vessels, nervous tissue, joints, the gastrointestinal tract (e.g., the stomach, and the large and small intestine), the liver, the spleen, the lungs, and the kidneys.

### Methods of Diagnosing;

### Diagnostic Methods for Patient Identification

For convenience, the diagnostic and/or patient identification methods and treatment methods resulting therefrom that are described in this section and other pans herein use a single formula, such as “Formula (I),” by way of example. In addition, the diagnostic and/or patient identification methods and treatment methods resulting therefrom described herein apply equally well to all formulas, or combinations or mixtures of formulas or agents, presented herein, that fail within the scope of Formula (I), FLAP inhibitors, NO modulators, as well as to all of the specific compounds that fall within the scope of these general categories.

The screening of “leukotriene-responsive patients” which are selected for treatment with a compound of Formula (I), a FLAP inhibitor or an NO modulator, or pharmaceutical compositions, or medicaments described herein which include a compound of Formula (I), a FLAP inhibitor or an NO modulator, is accomplished using techniques and methods described herein. Such techniques and methods include, by way of example, evaluation of gene haplotypes (genotype analysis), monitoring/measurement of biomarkers (phenotype analysis), monitoring/measurement of functional markers (phenotype analysis), which indicate patient response to known modulators of the leukotriene pathway, or any combination thereof.

### Genotype Analysis: FLAP Polymorphisms

Human FLAP has been purified and cloned and is an 18 kilodalton membrane-bound protein which is most highly expressed in human neutrophils. The FLAP gene is located at 13q12 and the gene has been linked to increased risk for both myocardial infarction and stroke in several populations. A number of polymorphisms and haplotypes in the gene encoding FLAP have been identified in, individuals (U.S. Patent Application 2005113408; Savers, Clin. Exp. Allergy, 33(8): 1103-10, 2003; Kedda, et al., Clin. Exp. Allergy, 35(3):332-8, 2005). Particular FLAP haplotypes have been linked to myocardial infarction and stroke in several populations (Helgadottir A et al Nature Genet 36:233-239 (2004); Helgadottir A et al Am J Hum Genet 76:505-509 (2004); Lohraussaar E et al Stroke 36:731-736 (2005): Kajimoto K et al Circ J 69:1029-1034 (2005). Previously, polymorphisms in certain genes have been demonstrated to correlate with responsiveness to given therapies, for example, the responsiveness of cancers to particular chemotherapeutic agents (Erichsen, et al., Br. J. Cancer, 90(4):747-51, 2004; Sullivan, et al. Oncogene, 23(19):3328-37, 2004). Therefore, patients who are under consideration for treatment with the combination compounds and therapies described herein may be screened for potential responsiveness to treatment based on their FLAP polymorphisms, or haplotypes.

Additionally, polymorphisms in any of the synthetic or signaling genes dedicated to the leukotriene pathway could result in a patient who is more responsive or less responsive to leukotriene modulator therapy (either FLAP or 5-LO inhibitor or leukotriene receptor antagonists). The genes dedicated to the leukotriene pathway are 5-lipoxygenase, 5-lipoxygenase-activating protein, LTA₄ hydrolase, LTC₄ synthase, LTB₄ receptor 1 (BLT₁), LTB₄ receptor 2 (BLT₂), cysteinyl leukotriene receptor 1 (CysLT₁R), cysteinyl leukotriene receptor 2 (CysLT₂R). For example, the 5-LO gene has been linked to aspirin intolerant asthma and airway hyperresponsiveness (Choi J H et al Hum Genet 114:337-344 (2004); Kim, S H et al Allergy 60:760-765 (2005). Genetic variants in the promoter region of 5-LO have been shown to predict clinical responses to a 5LO inhibitor in asthmatics (Drazen et al, Nature Genetics, 22, p 168-170, (1999), The LTC₄ synthase gene has been linked to atopy and asthma (Moissidis I et al Genet Meet 7:406-410 (2005). The CysLT₂ receptor has been linked to asthma and atopy (Thompson M D et al Pharmacogenetics 13:641-649 (2003): Pillai S G et al Pharmacogenetics 14:627-633 (2004); Park J S et al Pharmacogenet Genomics 15:483-492 (2005); Fukai H et al Pharmacogenetics 14:683-690 (2004), Any polymorphisms in arty leukotriene pathway gene or combination of polymorphisms or haplotypes may result in altered sensitivity of the patient to therapy aimed at reducing the pathological effects of leukotrienes. Selection of patients who might best respond to the leukotriene modulator therapies described herein may include knowledge of polymorphisms in the leukotriene pathway genes and also knowledge of the expression of leukotriene-driven mediators. Patient selection could be made on the basis of leukotriene pathway genotype alone, phenotype alone (biomarkers or functional markers) or any combination of genotype and phenotype.

A “haplotype,” as described herein, refers to a combination of genetic markers (“alleles”). A haplotype can comprise one or more alleles (e.g., a haplotype containing a single SNP), two or more alleles, three or more alleles, four or more alleles, or five or more alleles. The genetic markers are particular “alleles” at “polymorphic sites” associated with FLAP. A nucleotide position at which more than one sequence is possible in a population is referred to herein as a “polymorphic site.” Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. The term “variant FLAP” as used herein, refers to a sequence that differs from a reference FLAP sequence, but is otherwise substantially similar. The genetic markers that make up the haplotypes described herein are FLAP variants. In certain embodiments the FLAP variants are at least about 90% similar to a reference sequence. In other embodiments the FLAP variants are at least about 91% similar to a reference sequence. In other embodiments the FLAP variants are at least about 92% similar to a reference sequence. In other embodiments the FLAP variants are at least about 93% similar to a reference sequence. In other embodiments the FLAP variants are at least about 94% similar to a reference sequence. In other embodiments the FLAP variants are at least about 95% similar to a reference sequence. In other embodiments the FLAP variants are at least about 96% similar to a reference sequence. In other embodiments the FLAP variants are at least about 97% similar to a reference sequence. In other embodiments the FLAP variants are at least about 98% similar to a reference sequence. In other embodiments the FLAP variants are at least about 99% similar to a reference sequence.

Additionally, in certain embodiments the FLAP variants differ from the reference sequence by at least one base, while in other embodiments the FLAP variants differ from the reference sequence by at least two bases. In other embodiments the FLAP variants differ front the reference sequence by at least three bases, and in still other embodiments the FLAP variants differ from the reference sequence by at least four bases.

Additional variants can include changes that affect a polypeptide, e.g., the FLAP polypeptide. The polypeptide encoded by a reference nucleotide sequence is the “reference” polypeptide with a particular reference amino acid sequence, and polypeptides encoded by variant alleles are referred to as “variant” polypeptides with variant amino acid sequences. The FLAP nucleic acid sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence; duplication of all or a part of a sequence; transposition; or a rearrangement of a nucleotide sequence, as described in detail above. Such sequence changes alter the polypeptide encoded by a FLAP nucleic acid. For example, if the change in the nucleic acid sequence causes a frame shift, the frame shift can result in a change in the encoded amino acids, and/or can result in the generation of a premature stop codon, causing generation of a truncated polypeptide.

By way of example, a polymorphism associated with a susceptibility to myocardial infarction (MI), acute coronary syndrome (ACS), stroke or peripheral arterial occlusive disease (PAOD) can be a synonymous change in one or more nucleotides (i.e., a change that does not result in a change in the amino acid sequence). Such a polymorphism can, for example, alter splice sites, decrease or increase expression levels, affect the stability or transport of mRNA, or otherwise affect the transcription or translation of the polypeptide. The haplotypes described below are found more frequently in individuals with MI, ACS, stroke or PAOD than in individuals without MI, ACS, stroke or PAOD. Therefore, these haplotypes may have predictive value for detecting a susceptibility to MI, ACS, stroke or PAOD in an individual.

Several variants of the FLAP gene have been reported to correlate with the incidence of myocardial infarction in patients (Hakonarson, JAMA, 293(18):2245-56, 2005), plus FLAP gene markers reportedly associated with the risk for developing asthma have been described in U.S. Pat. No. 6,531,279. Methods for identifying FLAP sequence variants are described, e.g., in U.S. Publication No, 2005/0113408, and in U.S. Pat. No. 6,531,279, incorporated herein by reference herein in their entirety.

By way of example only, a haplotype associated with a susceptibility to myocardial infarction or stroke comprises markers SG13S99, SG13S25, SG13S377, SG13S106, SG13S32 and SG13S35 at the 13q12-13 locus. Or, the presence of the alleles T, G, G, G, A and G at SG13S99, SG13S25, SG13S377, SG13S106, SG13S32 and SG13S35, respectively (the B6 haplotype), is diagnostic of susceptibility to myocardial infarction or stroke. Or, a haplotype associated with a susceptibility to myocardial infarction or stroke comprises markers SG13S99, SG13S25, SG13S106, SG13S30 and SG13S42 at the 13q12-13 locus. Or, the presence of the alleles T, G, G, G and A at SG13S99, SG13S25, SG13S106, SG13S30 and SG13S42, respectively (the B5 haplotype), is diagnostic of susceptibility to myocardial infarction or stroke Or, a haplotype associated with a susceptibility to myocardial infarction or stroke comprises markers SG13S25, SG13S106, SG13S30 and SG13S42 at the 13q12-13 locus. Or, the presence of the alleles G, G, G and A at SG13S25, SG13S106, SG13S30 and SG13S42, respectively (the B4 haplotype), is diagnostic of susceptibility to myocardial infarction or stroke. Or, a haplotype associated with a susceptibility to myocardial infarction, or stoke comprises markers SG13S25, SG13S106, SG13S30 and SG13S32 at the 13q12-13 locus. Or, the presence of the alleles G, G, G and A at SG13S25, SG13S106, SG13S30 and SG13S32, respectively (the Bs4 haplotype), is diagnostic of susceptibility to myocardial infarction or stroke. In such embodiments just described, patients who are under consideration for treatment with combination compounds and therapies described herein, may be screened for potential responsiveness to treatment with combination compounds and therapies described herein based on such haplotypes.

By way of example only, a haplotype associated with a susceptibility to myocardial infarction or stroke comprises markers SG13S99, SG13S25, SG13S114, SG13S89 and SG13S32 at the 13q12-13 locus. Or, the presence of the alleles T, G, T, G and A at SG13S99, SG13S25, SG13S114, SG13S89 and SG13S32, respectively (the A5 haplotype), is diagnostic of susceptibility to myocardial infarction or stroke. Or, a haplotype associated with a susceptibility to myocardial infarction or stroke comprises markers SG13S25, SG13S114, SG13S89 and SG13S32 at the 13q12-13 locus. Or, the presence of the alleles G, T, G and A at SG13S25, SG13S114, SG13S89 and SG13S32, respectively (the A4 haplotype), is diagnostic of susceptibility to myocardial infarction or stroke, in such embodiments just described, patients who are under consideration for treatment with combination compounds and therapies described herein, may be screened for potential responsiveness to treatment with combination compounds and therapies described herein based on such haplotypes.

Detecting haplotypes can be accomplished by methods known in the art for detecting sequences at polymorphic sites, and therefore patients may be selected using genotype selection of FLAP, 5-LO or other leukotriene pathway gene polymorphisms. The presence or absence of a leukotriene pathway gene polymorphism or haplotype can be determined by various methods, including, for example, using enzymatic amplification, restriction fragment length polymorphism analysis, nucleic acid sequencing, electrophoretic analysts of nucleic acid from the individual, or any combination thereof, in certain embodiments, determination of a SNP or haplotype may identify patients who will respond to, or gain benefit from, treatment with compounds, of formula (I). By way of example, methods of diagnosing a susceptibility to myocardial infarction or stroke in an individual, comprises determining the presence or absence of certain single nucleotide polymorphisms (SNPs) or of certain haplotypes, wherein the presence of the SNP or the haplotype is diagnostic of susceptibility to myocardial infarction or stroke.

A variant of the gene encoding LTA₄ hydrolase has been shown to be associated with the increase in risk for myocardial infarction; Helgadottir et al. Nature Genetics, published on-line 2005. The haplotype (hapK) spanning the LTA-4H gene encoding LTA₄ hydrolase, in three cohorts from the United States, confers a modest risk (1.16) in an European American population but it confers a threefold larger risk in African Americans.

### Phenotype Analysis Biomarkers

In one aspect, patients who are under consideration for treatment with combination compounds and therapies described herein, are screened for potential responsiveness to treatment based on leukotriene-driven inflammatory biomarker phenotypes.

Patient screening based, on leukotriene-driven inflammatory biomarker phenotypes is used as an alternative to, or it may be complimentary with, patient screening by leukotriene pathway gene haplotype detection. The terra “biomarker” as used herein refers to a characteristic which can be measured and evaluated as an indicator of normal biological processes, pathological processes, or pharmacological responses to therapeutic intervention. Thus a biomarker is any substance, structure or process which is measured in the body, or its products, and which influences or predicts the incidence of outcome or disease. Biomarkers are classified into markers of exposure, effect, and susceptibility. In one aspect, biomarkers are physiologic endpoints, by way of example blood pressure, or they are analytical endpoints, by way of example, blood glucose, or cholesterol concentrations. Techniques, used to monitor and/or measure biomarkers include, but are not limited to, NMR, LC-MS, LC-MS/MS, GC-MS, GC-MS/MS, HPLC-MS, HPLC-MS/MS, FT-MS, FT-MS/MS, ICP-MS, ICP-MS/MS, peptide/protein sequencing, nucleic acid sequencing, electrophoresis techniques, immuno-assays, immuno-blotting, in-situ hybridization, fluorescence in-situ hybridization, PGR, radio-immuno assays, and enzyme-immuno assays. Single nucleotide polymorphisms (SNPs) have also been useful for the identification of biomarkers for propensity to certain diseases and also susceptibility or responsiveness to drugs such as chemotherapeutic agents and antiviral agents. These techniques, or any combination thereof, are used to screen patients for leukotriene-dependent or leukotriene mediated diseases or conditions, wherein such patients are beneficially treated with combination compounds and therapies described herein.

By way of example only, patients are selected for treatment with compounds of Formula (I), or drug combinations described herein that include compounds of Formula (I), by screening for enhanced inflammatory blood biomarkers such as, but not limited to, stimulated LTB₄, LTC₄, LTE₄, myeloperoxidase (MPO), eosinophil peroxidase (EPO), C-reactive protein (CRP), soluble intracellular adhesion, molecule (sICAM), monocyte chemoattractant protein (MCP-1), monocyte inflammatory protein (MIP-1α), interleukin-6 (IL-6), the TH2 T cell activators interleukin 4 (IL-4), and 13 (IL-13) and other inflammatory cytokines. In certain embodiments, patients with inflammatory respiratory diseases, including but not limited to, asthma and COPD, or with cardiovascular diseases, are selected as those most likely to be responsive to leukotriene synthesis inhibition using compounds of Formula (I) by using a panel of leukotriene driven inflammatory biomarkers.

### Phenotype Analysis: Functional Markers

Patients who are under consideration for treatment with combination compounds and therapies described herein, may be screened for response to known modulators of the leukotriene pathway. Patient screening by evaluation of functional markers as indicators of a patient's response to known modulators of the leukotriene pathway may be used as an alternative to, or it may be complimentary with, patient screening by leukotriene pathway gene haplotype detection (genotype analysis) and/or monitoring/measurement of leukotriene-driven inflammatory biomarker phenotypes. Functional markers may include, but are not limited to, any physical characteristics associated with a leukotriene dependent condition or disease, or knowledge of current or past drug treatment regimens.

By way of example only, the evaluation of lung volume and/or function is used as a functional marker for leukotriene-dependent or leukotriene mediated diseases or conditions, such as respiratory diseases. Its one aspect, lung function tests are used to screen patients, with such leukotriene-dependent or leukotriene mediated diseases or conditions, for treatment using compounds of Formula (I) or pharmaceutical compositions or medicaments which include compounds of Formula (I). Such tests include, but are not limited to, evaluation of lung volumes and capacities, such as tidal volume, inspiratory reserve volume, expiratory reserve volume, residual volume, inspiratory capacity, functional residual capacity, vital capacity, total king capacity, respiratory minute volume, alveolar ventilation, timed vital capacity, and ventilatory capacity. Method of measurement of lung volumes and capacities include, but are not limited to, maximum expiratory flow volume curve, forced expiratory volume in 1 sec. (FEV1), peak expiratory flow rate. In addition, other lung function tests used as functional markers for patient evaluation described herein include, but are not limited to, respiratory muscle power, maximum inspiratory pressure, maximum expiratory pressure, transdiaphragmatic pressure, distribution of ventilation, single breath nitrogen test, pulmonary nitrogen washout, and gas transfer.

Additionally, the knowledge of a patients past or current treatment regimen may be used as a functional marker to assist in screening patients for treatment of leukotriene dependent conditions or diseases using compounds of Formula (I) or pharmaceutical compositions or medicaments which include compounds of Formula (I). By way of example only, such treatment regimens may include past or current treatment using zileuton (Zyflo™), montelukast (Singulair™), pranlukast (Onon™), zafirlukast (Accolate™).

In some embodiments, patients who are under consideration for treatment with combination compounds and therapies described herein, are screened for functional markers which include, but are not limited to, reduced eosinophil and/or basophil, and/or neutrophil, and or monocyte and/or dendritic cell and/or lymphocyte recruitment, decreased mucosal secretion, decreased mucosal edema, and/or increased bronchodilation.

Methods for the identification of a patient in need of treatment for leukotriene-dependent or leukotriene mediated conditions or diseases, include methods wherein a patient sample is analyzed and the information obtained is used to identify possible treatment methods. It is expected that one skilled in the art will use this information in conjunction with other patient information, including, but not limited to age, weight, sex, diet, and medical condition, to choose a treatment method. It is also expected that each piece of information will be given a particular weight in the decision process. In certain embodiments, the information obtained from the diagnostic methods described above and any other patient information, including, but not limited to age, weight, sex, diet, and medical, condition, are incorporated into an algorithm used to elucidate a treatment method, wherein each piece of information will be given a particular weight in the decision process.

In another embodiment, FLAP NO combination therapy is particularly useful in patients with respiratory disease including asthma whom have been selected by haplotype or leukotriene-responsive phenotype. FLAP single nucleotide polymorphisms and/or specific haplotypes that have been linked to asthma are described fully in US patent to Blumenfeld et al, Genset S. A. U.S. Pat. No. 6,531,279 B1. In addition, polymorphisms and/or specific haplotypes in any of the committed genes in the leukotriene synthetic and signaling pathway may predispose a specific patient towards greater or lesser responsiveness to FLAP inhibition.

### Combination Therapies:

The present disclosure features a combination therapy comprising administering one or more NO modulators (including agents that release NO in a patient or agents that otherwise raise NO in a patient or, alternatively, maintain production of NO or agents that otherwise maintain levels of NO in a patient), and one or more FLAP inhibitors for the treatment of diseases, conditions or disorders, as described herein, or associated symptoms or complications thereof. Since NO modulators (including agents that release NO in a patient or agents that otherwise raise NO in a patient or, alternatively, maintain production of NO or agents that otherwise maintain levels of NO in a patient) are expected to have a mechanism of action different from that of FLAP inhibitors, the disclosed combination with FLAP inhibitors has the advantage of potentially reducing the amount of either drug necessary to achieve combined therapeutic or pharmaceutical efficacy, relative to the use of either drug alone, thereby reducing one or more adverse side-effects. Further when such combination compositions or therapies are used in former combination with the diagnostic analyses described herein, the particular combination composition or therapy used to heat that patient can be optimized.

The magnitude of a therapeutic dose of each active ingredient (including compounds of Formula I, or combinations of FLAP inhibitors and NO modulators, or combinations thereof) will vary with the disorder itself, the specific active ingredients, and the route of administration. The dose and/or dose frequency will also vary according to age, body weight, response, and the past medical history of the patient. Suitable dosing regimens can be selected by those skilled in the art with due consideration of such factors by following, for example, dosages reported in the literature and recommended in the Physician's Desk Reference® (54th ed., 2000).

The combination therapies described herein can be administered for prophylactic and/or therapeutic treatments. The term “treating” is used to refer to either prophylactic and/or therapeutic treatments. In therapeutic applications, the compositions are administered to a patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician. A person of skill in the art can determine therapeutically effective amounts by experience with other or similar agents, reference to the published literature, by experimentation, (e.g., a dose escalation clinical trial), or by a combination of such methods.

In prophylactic applications, the combination therapies described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. A person of skill in the art can determine therapeutically effective amounts by experience with other or similar agents, reference to the published literature, by experimentation (e.g., a dose escalation clinical trial), or by a combination of such methods.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition. In the case wherein the patient's status does improve, upon the doctor's discretion the administration of the compounds may be given continuously; alternatively, the dose of drug being administered may be temporarily reduced or temporarily suspended for a certain length of time (i.e., a “drug holiday”), The length of the drug holiday can vary between 2 days and 1 year, and the dose reduction during a drug holiday may be from 10%-100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The concept of the combination therapies described herein embraces the administration of one or more FLAP inhibitors, and one or more NO modulators (including agents that release NO in a patient or agents that otherwise raise NO in a patient or, alternatively, maintain production of NO or agents that otherwise maintain levels of NO in a patient) as part of a specific treatment regimen intended to provide a beneficial effect from the co-action of these therapeutic agents. For convenience, a combination therapy that combines administration of at least one FLAP inhibitor and at least one NO modulator (including agents that release NO in a patient or agents that otherwise raise NO in a patient or, alternatively, maintain production of NO or agents that otherwise maintain levels of NO in a patient) will be referred to as NO-FLAP therapy. In the NO-FLAP therapies described herein, the NO modulator (including agents that release NO in a patient or agents that otherwise raise NO in a patient or, alternatively, maintain production of NO or agents that otherwise maintain levels of NO in a patient) and the FLAP inhibitor can be administered simultaneously or sequentially. When administered simultaneously, the NO modulator (including agents that release NO in a patient or agents that otherwise raise NO in a patient or, alternatively, maintain production of NO or agents that otherwise maintain levels of NO in a patient) and the FLAP inhibitor can be chemically linked, physically admixed or provided in two separate pharmaceutical compositions. The combination compositions described herein include NO modulators (including agents that release NO in a patient or agents that otherwise raise NO in a patient or, alternatively, maintain production of NO or agents that otherwise maintain levels of NO in a patient) and FLAP inhibitors that are chemically linked to form a compound, provided that such chemically linkage can occur either prior to or subsequent to administration to the patient; also included as combination compositions are pharmaceutical compositions comprising either a chemically linked compound, as described above, or chemically unlinked, but physically admixed agents.

The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). The combination therapies described herein are generally not intended to encompass the administration of two or more of these therapeutic agents as part of separate monotherapy regimens that incidentally and arbitrarily result in the combinations of the present invention. Rather, combination therapy is intended to embrace administration of these therapeutic agents in a simultaneous or sequential manner, that is, wherein each therapeutic agent is administered at the same time or at a different time. Simultaneous administration includes, for example, administering to the subject a single capsule having a fixed ratio of each therapeutic agent or in multiple, single capsules for each of the therapeutic agents. Simultaneous administration can be accomplished, for example, by administering a capsule containing one or more NO modulators (including agents that release NO in a patient or agents that otherwise raise NO in a patient or, alternatively, maintain production of NO or agents that otherwise maintain levels of NO in a patient) and a capsule containing one or more FLAP inhibitors at the same time. Sequential administration of each therapeutic agent can be accomplished, for example, by administering a capsule containing one or more NO modulators (including agents that release NO in a patient or agents that otherwise raise NO in a patient or, alternatively, maintain production of NO or agents that otherwise maintain levels of NO in a patient) at one time followed by administering a capsule containing one or more FLAP inhibitors at a different time.

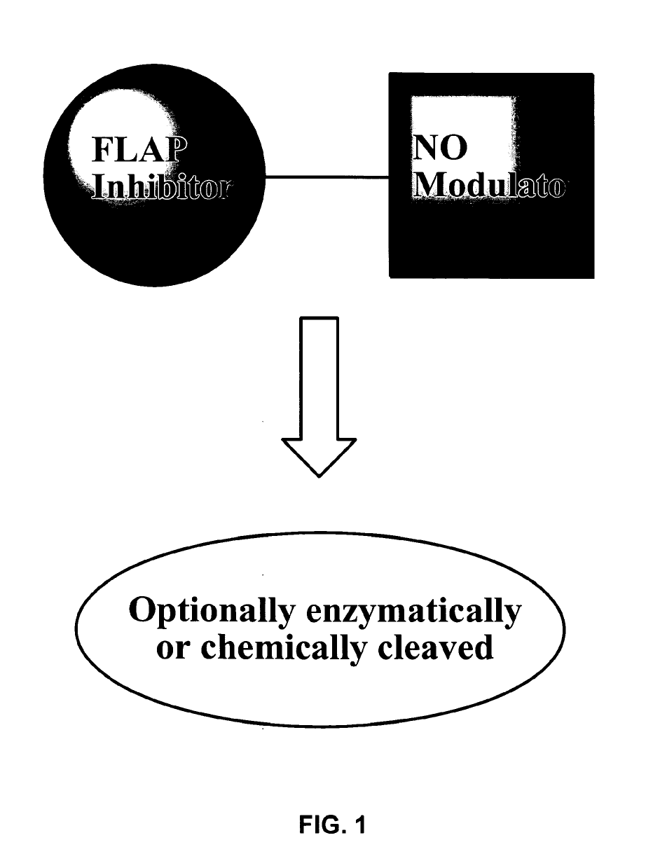

FIG. 1 presents a possible embodiment for the combination compositions and therapies described herein. In FIG. 1, the NO-FLAP therapy is accomplished via chemically linked agents—the line drawn between the two agents in FIG. 1 represents any form of chemically linkage, including a linker “L,” covalent bonds, ionic bonds, hydrogen bonds, van der Waals interactions, or any combination of the foregoing. Further, the chemical linkage does not have to be directly between the two agents, but rather a bridging group, comprised of non-therapeutically active moieties, can serve as the chemical linkage, i.e., (FLAP inhibitor)-(bridging group)-(NO modulator). Further, the chemically linked combination composition optionally may be cleaved in vivo to form two unlinked therapeutically active agents. The cleavage reactions) can occur either enzymatically (or by some other biological agent), chemically (including the cleavage of the two linked agents by another compound that has been administered to the patient), electrochemically, photochemically, sonochemically, via another activation mechanism, or any combination of the above. Further the cleavage reaction(s) can occur site specifically, via targeted enzymatic reactions (including targeted esterases), or the cleavage reaction(s) can occur via generally available pathways.

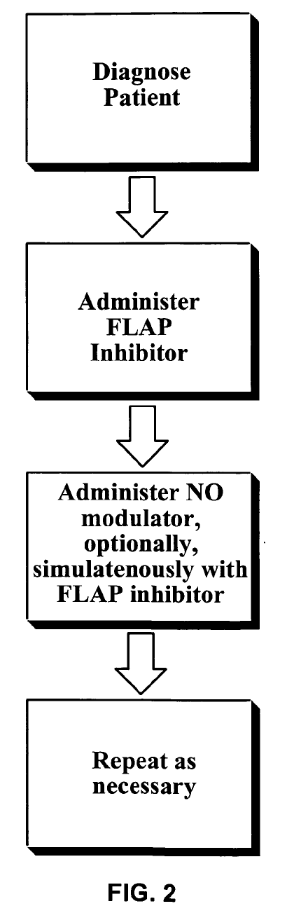

FIG. 2 presents one embodiment of the combination therapy described herein. In this approach, the patient is first diagnosed, a process which can optionally include determination of the patient's FLAP haplotype, polymorphisms in other leukotriene-pathway specific genes, use of biomarkers and leukotriene-responsive phenotypes, and/or use of functional markers. Based on the diagnostic information, as well as other information available to the physician, the patient is administered NO-FLAP therapy, which can include the simultaneous or sequential administration of the combination compositions (which may be chemically linked, physically admixed, or physically separate). As necessary, the NO-FLAP therapy is continued per discretion of the physician (and based on the patient's response to the therapy).

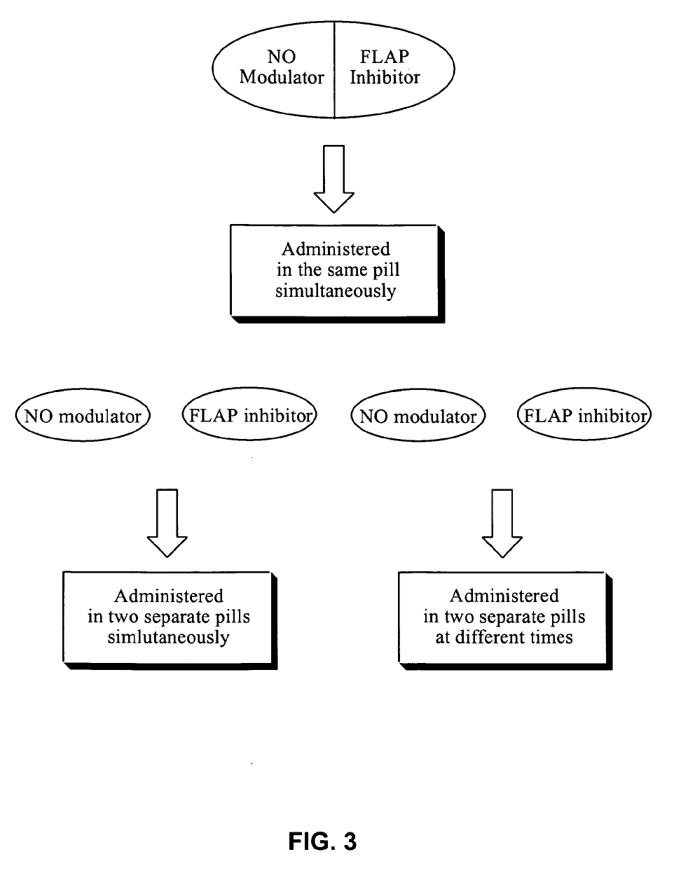

FIG. 3 presents three different embodiments of the NO-FLAP combination compositions and therapies described herein. It should be understood that when, administered in the same “pill,” the NO-FLAP combination composition includes both chemically linked and physically admixed agents. FIG. 3 describes one embodiment in which a “pill” form is utilized. However, it should be understood that the “pill” form is used for illustrative purposes only; any of the dosage forms described herein and/or those disclosed in the art (including by way of example only “Remington: The Science and Practice of Pharmacy,” 20th ed. (2000)).

Sequential or simultaneous administration of each therapeutic agent is effected by any appropriate route including, but not limited to, oral routes, intravenous routes, rectal routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination is administered orally. Alternatively, for example, all therapeutic agents are administered orally or all therapeutic agents are administered by intravenous injection. “Combination therapy” also embraces the administration of the therapeutic agents as described above in further combination with other biologically active ingredients.

In certain instances, it may be appropriate to administer at least one of the combination therapies described herein (or a pharmaceutically acceptable salt, ester, amide, prodrug, or solvate) in combination with another therapeutic agent. By way of example only, if one of the side effects experienced by a patient upon receiving one of the combination therapies herein is inflammation, then it may be appropriate to administer an additional anti-inflammatory agent in combination with the initial combination therapy. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced.) Or, by way of example only, the benefit experienced by a patient may be increased by administering one of the compounds described herein with another therapeutic agent (which also includes a therapeutic regimen) that also has therapeutic benefit. In one aspect, by way of example only, in a treatment for asthma involving administration of one of the compounds described herein, increased therapeutic benefit results by also providing the patient with other therapeutic agents or therapies for asthma. In any case, regardless of the disease, disorder or condition being treated, the overall benefit experienced by the patient may simply be additive of the combination therapy and therapeutic agent or the patient may experience a synergistic benefit; both additive and synergistic benefits are encompassed by the combination methods and therapies described herein.

In one embodiment described herein, methods for treatment of leukotriene-dependent or leukotriene mediated conditions or diseases include administering to a patient a compound of Formula (I), FLAP inhibitor and/or NO modulator in combination with other therapeutic agents that are used in the treatment of respiratory conditions or disorders, such as, but not limited to asthma. Therapeutic agents used in the treatment of respiratory conditions and disorders, such as, but not limited to asthma, include: glucocorticoids, such as, ciclesonide, beclomethasone, budesonide, flunisolide, fluticasone, mometasone, and triamcinolone; leukotriene modifiers, such as, montelukast, zafirlukast, pranlukast, and zileuton; mast cell stabilizers, such as, cromoglicate (cromolyn), and nedocromil; antimuscarinics/anticholinergics, such as, ipratropium, oxitropium, and tiotropium; methylxanthines, such as, theophylline and aminophylline; antihistamine, such as, mepyramine (pyrilamine), antazoline, diphenhydramine, carbinoxamine, doxylamine, clemastine, dimenhydrinate, pheniramine, chlorphenamine (chlorpheniramine), dexchlorphenamine, brompheniramine, triprolidine, cyclizine, chlorocyclizine, hydroxyzine, meclizine, promethazine, alimemazine (trimeprazine), cyproheptadine, azatadine, ketotifen, acrivastine, astemizole, cetirizine, loratadine, mizolastine, terfenadine, fexofenadine, levocetirizine, desloratadine, fexofenadine; omalizumab, an IgF blocker; beta2-adrenergic receptor agonists, such as: short acting beta2-adrenergic receptor agonists, such as, salbutamol (albuterol), levalbuterol, terbutaline, pirbuterol, procaterol, metaproterenol, fenoterol, bitolterol mesylate; and long-acting beta2-adrenergic receptor agonists, such as, salmeterol, formoterol, bambuterol.

In one embodiment described herein, methods for treatment, of leukotriene-dependent or leukotriene mediated conditions or diseases include administering to a patient a compound of Formula (I), FLAP inhibitor and/or NO modulator in combination with one or more agents used to treat used to treat asthma, including, but not limited to: combination Inhalers (fluticasone and salmeterol oral inhalation (e.g. Advair)); inhaled Beta-2 agonists (albuterol inhaler; albuterol nebulizer solution; formoterol; isoproterenol oral inhalation; levalbuterol; metaproterenol inhalation; pirbuterol acetate oral inhalation; salmeterol aerosol inhalation; salmeterol powder inhalation; terbutaline inhaler); inhaled corticosteroids (beclomethasone oral inhalation; budesonide inhalation solution; budesonide inhaler; flunisolide oral inhalation; fluticasone inhalation aerosol; fluticasone powder for oral inhalation; mometasone inhalation powder; triamcinolone oral inhalation); leukotriene modifiers (montelukast; zafirlukast; zileuton); mast cell, stabilizers (cromolyn inhaler; nedocromil oral inhalation); monoclonal antibodies (omalizumab); oral Beta-2 agonists (albuterol oral syrup; albuterol oral tablets; metaproterenol; terbutaline); bronchodilator (aminophylline; oxtriphylline; theophylline).

In one embodiment described herein, methods for treatment of leukotriene-dependent or leukotriene mediated conditions or diseases include administering to a patient a compound of Formula (I), FLAP inhibitor and/or NO modulator in combination with one or more agents used to treat allergy, including, but not limited to: antihistamine and decongestant combinations (cetirizine and pseudoephedrine; desloratadine and pseudoephedrine ER; fexofenadine and pseudoephedrine; loratadine and pseudoephedrine); antihistamines (azelastine nasal spray; brompheniramine; brompheniramine oral suspension; carbinoxamine; cetirizine; chlorpheniramine; clemastine; desloratadine; dexchlorpheniramine ER; dexchlorpheniramine oral syrup; diphenhydramine oral; fexofenadine; loratadine; promethazine); decongestants (pseudoephedrine); leukotriene modifiers (montelukast; montelukast granules); nasal anticholinergics (ipratropium); nasal corticosteroids (beclomethasone nasal inhalation; budesonide nasal inhaler; flunisolide nasal inhalation; fluticasone nasal inhalation; mometasone nasal spray; triamcinolone nasal inhalation; triamcinolone nasal spray); nasal decongestants (phenylephrine); nasal mast cell stabilizers (cromolyn nasal spray).

In one embodiment described herein, methods for treatment of leukotriene-dependent or leukotriene mediated conditions or diseases include administering to a patient a compound of Formula (I), FLAP inhibitor and/or NO modulator in combination with one or more agents used to treat chronic obstructive pulmonary disease (COPD), including, but not limited to: anticholinergics-ipratropium bromide oral inhalation); combination Inhalers (albuterol and ipratropium (e.g. Combivent, DuoNeb); fluticasone and salmeterol oral inhalation (e.g. Advair)); corticosteroids (dexamethasone tablets; fludrocortisone acetate; hydrocortisone tablets; methylprednisolone; prednisolone liquid; prednisone oral; triamcinolone oral); inhaled Beta-2 Agonists (albuterol inhaler; albuterol nebulizer solution; formoterol; isoproterenol oral inhalation; levalbuterol; metaproterenol inhalation; pirbuterol acetate oral inhalation; salmeterol aerosol inhalation; salmeterol powder inhalation; terbutaline inhaler); inhaled Corticosteroids (beclomethasone oral inhalation; budesonide inhalation solution; budesonide inhaler; flunisolide oral inhalation; fluticasone inhalation aerosol; fluticasone powder for oral inhalation; triamcinolone oral inhalation); mucolytics (guaifenesin); oral Beta-2 agonists (albuterol oral syrup; albuterol oral tablets: metaproterenol; terbutaline); bronchodilator (aminophylline; oxtriphylline; theophylline).

In one embodiment described herein, methods for treatment of leukotriene-dependent or leukotriene mediated conditions or diseases include administering to a patient a compound of Formula (I), FLAP inhibitor and/or NO modulator in combination with an anti-inflammatory agent including, but not limited to non-steroidal anti-inflammatory drugs (NSAIDs) and corticosteroids (glucocorticoids).

NSAIDs include, but are not limited to: aspirin, salicylic acid, gentisic acid, choline magnesium salicylate, choline salicylate, choline magnesium salicylate, choline salicylate, magnesium salicylate, sodium salicylate, diflunisal, carprofen, fenoprofen, fenoprofen calcium, flurbiprofen, ibuprofen, ketoprofen, nabutone, ketolorac, ketorolac tromethamine, naproxen, oxaprozin, diclofenac, etodolac, indomethacin, sulindac, tolmetin, meclofenamate, meclofenamate sodium, mefenamic acid, piroxicam, meloxicam, COX-2 specific inhibitors (such as, but not limited to, celecoxib, rofecoxib, valdecoxib, parecoxib, etoricoxib, lumiracoxib, CS-502, JTB-522, L-745,337 and NS398).

Corticosteroids, include, but are not limited to: betamethasone (Celestone), prednisone (Deltasone), alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, deoxycortone, dexamethasone, diflorasone, diflucortolone, difluprednate, fluclorolone, fludrocortisone, fludroxycortide, flumethasone, flunisolide, fluocinolone acetonide, fluocinonide, fluocortin, fluocortolone, fluorometholone, fluperolone, fluprednidene, fluticasone, formocortal, halcinonide, halometasone, hydrocortisone/cortisol, hydrocortisone aceponate, hydrocortisone buteprate, hydrocortisone butyrate, loteprednol, medrysone, meprednisone, methylprednisolone, methylprednisone aceponate, mometasone furoate, paramethasone, prednicarbate, prednisone/prednisolone, rimexolone, tixocortol, triamcinolone, and ulobetasol.

Corticosteroids do not directly inhibit leukotriene production, therefore co-dosing with steroids, in one embodiment, provide additional anti-inflammatory benefit.

In other embodiments described herein, methods for treatment of leukotriene-dependent or leukotriene mediated conditions or diseases include administering to a patient a compound of Formula (I). FLAP inhibitor and/or NO modulator in combination with an anti-inflammatory agent including, but not limited to poly-unsaturated fatty acids (PUFAs) such as docosahexanoic acid (DHA), eicosapentaenoic acid (EPA) and alpha-linolenic acid (ALA).

By way of another example of a combination therapy involving an additional agent are methods for treatment of leukotriene-dependent or leukotriene mediated conditions or diseases, such as atherosclerosis. Such methods comprise administering to a patient compounds, pharmaceutical compositions, or medicaments described herein in combination with at least one additional, agent selected from the group consisting of HMG-CoA reductase inhibitors (e.g., statins in their lactonized or dihydroxy open acid forms and pharmaceuticaily acceptable salts and esters thereof, including but not limited to lovastatin; simvastatin; dihydroxy open-acid simvastatin, particularly the ammonium or calcium salts thereof; pravastatin, particularly the sodium salt thereof; fluvastatin, particularly the sodium salt thereof; atorvastatin, particularly the calcium salt thereof; nisvastatin, also referred to as NK-104; rosuvastatin); agents that have both lipid-altering effects and other pharmaceutical activities; HMG-CoA synthase inhibitors; cholesterol absorption inhibitors such, as ezetimibe; cholesterol ester transfer protein (CETP) inhibitors, for example JTT-705 and CP529, 41.4; squalene epoxidase inhibitors; squalene synthetase inhibitors (also known as squalene synthase inhibitors); acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists, including the compounds commonly referred to as glitazones, for example troglitazone, pioglitazone and rosiglitazone and including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists such as 5-[(2,4-dioxo-thiazolidinyl)methyl]-2-methoxy-N-[[4-(trifluoromethyl)phenyl]methyl]-benzamide, known as KRP-297; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such, as losartan; angiotensin converting enzyme inhibitors such as enalapril and captopril; calcium channel blockers such, as nifedipine and diltiazem; endothelian antagonists; agents that enhance ABC1 gene expression; FXR and LXR ligands including both inhibitors and agonists; bisphosphonate compounds such as alendronate sodium; and cyclooxygenase-2 inhibitors such as rofecoxib and celecoxib.

In one embodiment described herein, a compound of Formula (I), FLAP inhibitor and/or NO modulator is administered or formulated in combination with antihypertensives, which include: diuretics (e.g. loop diuretics: bumetamide, ethacrynic acid, furosemide, torsemide; thiazide diuretics: chlortalidone, epitizide, hydrochlorothiazide and chlorothiazide, bendroflumethiazide; thiazide-like diuretics: indapamide, chlorthalidone, metolazone; potassium-sparing diuretics: amiloride, triamteren, spironolactone.); adrenergic receptor antagonists (e.g. beta blockers: atenolol, metoprolol, nadolol, oxprenolol, pindolol, propranolol, timolol; alpha blockers: doxazosin, phentolamine, indoramin, phenoxybenzamine, prazosin, terazosin, tolazoline; mixed alpha+beta blockers: bucindolol, carvedilol, labetalol); adrenergic receptor agonist (e.g. alpha-2 agonists: clonidine, methyldopa); calcium channel blockers (e.g. dihydropyridines: amlodipine, felodipine, isradipine, lercanidipine, nifedipine (Adalai®), nimodipine, Nitrendipine; non-dihydropyridines: diltiazem, verapamil); ACE inhibitors (e.g. captopril, enalapril, fosinopril, lisinopril, perindopril, quinapril, ramipril, trandopril, benzapril); angiotensin II receptor antagonists (e.g. candesartan, eprosartan, irbesartan, losartan, olmesartan, telmisartan, valsartan); aldosterone antagonists (e.g. eplerenone, spironolactone); vasodilators (e.g. sodium nitroprusside); centrally acting adrenergic drugs (e.g. Clonidine, Guanabenz, Methyldopa); adrenergic neuron blockers; Guanethidine, Reserpine.

In one embodiment, an analgesic and/or and anti-inflammatory compound is administered in combination to the compound of Formula (I), FLAP inhibitor, or NO modulator. Analgesic and/or and anti-inflammatory compounds include, but are not limited to: aspirin, carbaspirin, choline salicylate, diflunisal, magnesium salicylate, salicylamide, salicylic acid, salsalate, sodium thiosalicylate, acetaminophen, phenacetin, aminopyrine, mefenamic acid, methotrimeprazine, oxyphenbutazone, phenylbutazone, indomethacin, ibuprofen, sulindac, piroxicam, meclofenamate, zomepirac, codeine, morphine, meperidine, pethinine, alphaprodine, fentanyl, levorphanol, methadone, phenazocine, butorphanol, ethobeptozine, nalbuphine, pentazocine, propoxyphene, fenoprofen, naproxen, tolmeton and the like.

In another embodiment described herein, methods for treatment of leukotriene-dependent or leukotriene mediated conditions or diseases include administering to a patient a FLAP inhibitor in combination with NSAIDs and NO-donors or NSAIDs and proton-pump inhibitors, in one aspect, a compound of Formula (I) is administered with an NSAID.

Proton-pomp inhibitors include but are not limited to, omeprazole, lansoprazole, esomeprazole, pantoprazole, rabeprazole.

The combination compositions and therapies described herein include the use of compounds of Formula (I); FLAP inhibitors administered with (simultaneous or sequential) NO modulators; compounds of Formula (I) administered with (simultaneous or sequential) NO modulators; and compounds of Formula (I) administered with (simultaneous or sequential) FLAP inhibitors.

By way of example only, an exemplary order of administration of the combination therapy is as follows: the first agent being a NO modulator (including agents that release NO in a patient or agents that otherwise raise NO in a patient or, alternatively, maintain production of NO or agents that otherwise maintain levels of NO in a patient), the second agent being a FLAP inhibitor. Another exemplary order of administration of the combination therapy is as follows: the first agent being a FLAP inhibitor, the second agent being a NO modulator (including agents that release NO in a patient or agents that otherwise raise NO in a patient or, alternatively, maintain, production of NO or agents that otherwise maintain levels of NO in a patient). Another exemplary order of administration of the compounds is as follows: the first agent being a NO modulator (including agents that release NO in a patient or agents that otherwise raise NO in a patient or, alternatively, maintain production of NO or agents that otherwise maintain levels of NO in a patient), the second agent being a FLAP inhibitor, the third agent being a NO modulator (including agents that release NO in a patient or agents that otherwise raise NO in a patient or, alternatively, maintain production of NO or agents that otherwise maintain levels of NO in a patient). Another exemplary order of administration of the combination therapy is as follows: the first agent being a FLAP inhibitor, the second agent being a NO modulator (including agents that release NO in a patient or agents that otherwise raise NO in a patient or, alternatively, maintain production of NO or agents that otherwise maintain levels of NO in a patient), the third agent being a FLAP inhibitor. Another exemplary order of administration of the combination therapy is as follows: administration of a compound of Formula (I), followed after a period of lime by the administration of the same compound of Formula (I). By way of further or additional examples only: one agent is administered at the same time as another and before administration of the last agent; one agent is administered at the same time as another and after administration of the last agent; all three agents are administered at the same time; and specific agents are administered at multiple doses. In any of these examples, the described administrations can be repeated per the discretion of the physician based on the results of the patient to the therapy. If the therapy involves the multiple administration of an agent or agents as described herein, the timing between the multiple doses may vary from more than zero weeks to less than four weeks.

### Kits/Articles of Manufacture

For use in the combination therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

In one aspect, a kit will typically comprises one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. In one aspect, the label also indicates directions for use of the contents, such as in the methods described herein.

### EXAMPLES

The following examples provide illustrative methods for testing the effectiveness and safety of the compounds of Formula (I), the FLAP inhibitors, NO modulators, and combinations of FLAP inhibitors and NO modulators, and for forming pharmaceutical compositions of compounds of Formula (I), the FLAP inhibitors, NO modulators, and combinations of FLAP inhibitors and NO modulators. For convenience, the examples use a single formula, such as “Formula (I).” However, the examples apply equally well to all formulas presented herein that fall within the scope of Formula (I), FLAP inhibitors, NO modulators, and combinations of FLAP inhibitors and NO modulators, as well as to all of the specific compounds that fall within the scope of these generic formulae. These examples are provided for illustrative purposes only and not to limit the scope of the claims provided herein.

### Example 1a

### Synthesis of 3-[3-tert-Butylsulfanyl-1-(4-chloro-benzyl)-5-isopropyl-1H-indol-2-yl]-2,2-dimethyl-propionic acid (Compound 1-1a)

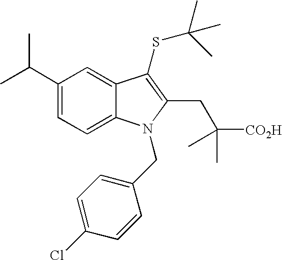

Prepared using the procedure described in U.S. Pat. No. 5,081,138, issued Jan. 4, 1992.

### Example 2a

### Synthesis of 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid (Compound 1-2a)

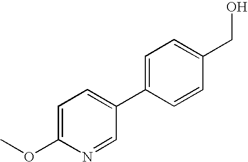

### Step 1: [4-(6-Methoxy-pyridin-3-yl)-phenyl]-methanol

To (4-hydroxymethylphenyl)boronic acid (Combi-Blocks; 10.0 g, 65.8 mmol) in DME/H₂O (60 mL, 2:1) was added 5-bromo-2-methoxypyridine (Combi-Blocks; 13.6 g, 72.4 mmol) and potassium carbonate (45.5 g, 328.9 mmol). The reaction was degassed with N₂ for 20 minutes. Tetrakis(triphenylphosphine)palladium(0) (0.760 g, 0.66 mmol) was added and the reaction was further degassed for 15 minutes. The reaction was then heated to 80° C. for 16 hours under N₂. LCMS confirmed the formation of the product. The reaction was partitioned between H₂O and EtOAc and the aqueous layer was extracted twice with EtOAc. The combined organic layers were dried over MgSO₄, filtered, concentrated, and triturated with EtOAc to give the desired product.

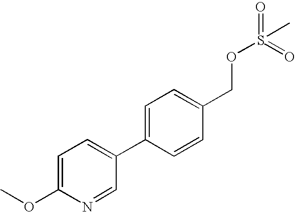

### Step 2: Methanesulfonic acid 4-(6-methoxy-pyridin-3-yl)-benzyl ester

[4-(6-Methoxy-pyridin-3-yl)-phenyl]-methanol (0.43 g, 2.0 mmol) was dissolved in CH₂Cl₂ (10 mL) and cooled to 0° C. under N₂. Triethylamine (44 uL, 3.2 mmol) was added, followed by methanesulfonyl chloride (170 uL, 2.2 mmol), and the reaction was stirred at 0° C. for 30 minutes, then at room temperature for 30 minutes. TLC analysis confirmed the formation of the product, so the reaction was partitioned between H₂O and CH₂Cl₂ and the aqueous phase was extracted twice with CH₂Cl₂. The combined organic layers were dried over MgSO₄, filtered, and concentrated to give the desired product as an off-white solid.

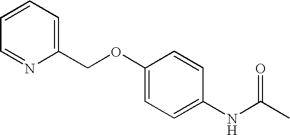

### Step 3: N-[4-(Pyridin-2-ylmethoxy)-phenyl]-acetamide

A mixture of 4-acetamidophenol (Sigma-Aldrich; 73.6 g), 2-chloromethylpyridine hydrochloride (80 g) and cesium carbonate (320 g) in DMF (1 L) was stirred at 70° C. for 2 days. The mixture was cooled, poured into H₂O (2 L) and extracted with EtOAc 6 times. The organic layers were washed with brine, dried over MgSO₄, and filtered to give a tan solid (114 g) which was used as such in the next step.

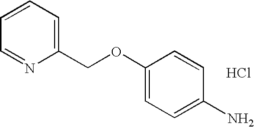

### Step 4: 4-(Pyridin-2-ylmethoxy)-phenylamine hydrochloride

To N-[4-(pyridin-2-ylmethoxy)-phenyl]-acetamide (114 g) in EtOH (1 L) was added potassium hydroxide (50 g) in H₂O (200 mL). The reaction was heated to 110° C. for 2 days, and then additional potassium hydroxide (20 g in 100 mL H₂O) was added and heating continued for a further 2 days. The solution was cooled to room temperature and concentrated, and the residue was partitioned between EtOAc and H₂O. The aqueous layer was extracted 3 times with EtOAc, and the combined organic layers were washed with brine, dried over MgSO₄ and filtered. To this solution was added saturated HCl in EtOAc and a precipitate formed immediately. Collection of the solids by filtration, followed by drying under vacuum, provided the title compound as a pink solid (95 g).

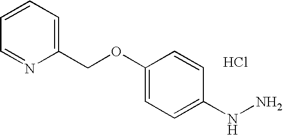

### Step 5: [4-(Pyridin-2-ylmethoxy)-phenyl]hydrazine dihydrochloride

To 4-(pyridin-2-ylmethoxy)-phenylamine hydrochloride (95 g) in H₂O (1 L) at 0° C. was added sodium nitrite (26 g) in H₂O (100 mL). The diazonium salt was allowed to form over 45 minutes and was then poured slowly into a rapidly stirring mixture of sodium hydrosulphite (350 g) in H₂O (1 L) and ether (1 L) at 0° C. over 15 minutes. Stirring continued for 40 minutes, and then the mixture was made basic using concentrated potassium hydroxide. After extraction using EtOAc twice, the combined organic layers were washed with H₂O and brine, dried over MgSO₄ and filtered. To this solution was added saturated HCl in EtOAc and a precipitate formed immediately. Collection of the solids by filtration, followed by drying under vacuum, provided the title compound as a tan solid (75 g).

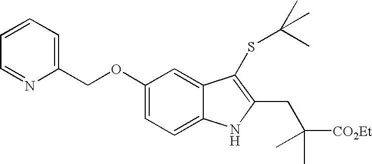

### Step 6: 3-[3-tert-Butylsulfanyl-5-(pyridin-2-ylmethoxy)-1H-indol-2-yl]-2,2-dimethyl-propionic acid ethyl ester

[4-(Pyridin-2-ylmethoxy)-phenyl]-hydrazine dihydrochloride (75 g), ethyl 5-(t-butylthio)-2,2-dimethyl-4-oxo-pentanoate (prepared according to the procedures described in U.S. Pat. No. 5,288,743 issued Feb. 22, 1994; 64 g), sodium acetate (40 g), and acetic acid (400 mL) were combined in toluene (800 mL) and stirred at room temperature for 3 days. The mixture was poured into H₂O and made basic with solid sodium carbonate. The mixture was extracted with EtOAc 3 times, and the combined organic layers were washed twice with H₂O, once with brine, dried over MgSO₄, filtered and concentrated to give a dark red-black oil. Purification by silica gel chromatography of the mother liquor (0 to 20% EtOAc in hexanes) afforded 68 g of the title compound as a yellow solid.

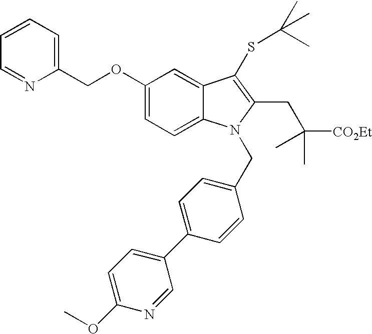

### Step 7: 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid ethyl ester

3-[3-tert-Butylsulfanyl-5-(pyridin-2-ylmethoxy)-1H-indol-2-yl]-2,2-dimethyl-propionic acid ethyl ester (Step 4; 0.73 g, 1.7 mmol) was dissolved in DMF (10 mL) and cooled to −10° C. under N₂. Sodium hydride (60% dispersion in mineral oil; 0.08 g, 2.0 mmol) was added portionwise, and the reaction was stirred at −10° C. for 45 minutes until the foam had disappeared. To this dark brown-reddish solution was added methanesulfonic acid 4-(6-methoxy-pyridin-3-yl)-benzyl ester in DMF (7 mL) dropwise. The reaction was then stirred at −10° C. for 1 hour and allowed to warm to room temperature slowly. After 16 hours, LCMS confirmed the formation of the product. The reaction was quenched with saturated NH₄Cl and diluted with methyl tert-butyl ether (MTBE) and H₂O. The aqueous phase was extracted twice with MTBE. The combined organic layers were dried over MgSO₄, filtered, and concentrated, and the crude product was purified by silica gel chromatography to give the desired product.

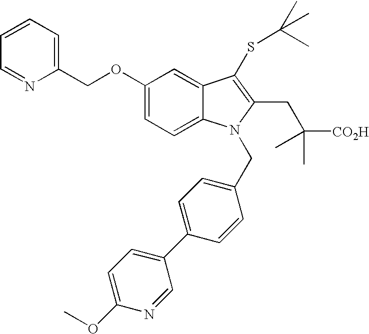

### Step 8: 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid

3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid ethyl ester (21.5 g, 33.7 mmol) was dissolved in THF (100 mL) and MeOH (100 mL) and stirred until it became a clear solution. Aqueous lithium hydroxide (3N; 56 mL, 168.5 mmol) was added and the reaction was refluxed at 80° C. for 2 hours. LCMS confirmed the formation of the product, so the reaction was cooled to room temperature and partitioned between EtOAc and H₂O. The pH of the aqueous solution was adjusted to pH 1 with aqueous 10% HCl, and the aqueous phase was extracted three times with EtOAc. The combined organic layers were washed with H₂O, dried over MgSO₄, filtered, and concentrated to give the desired free acid.

### Example 3a

### Synthesis of 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid (Compound 1-3a)

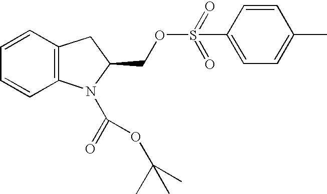

### Step 1: N—BOC—(S)-2-(toluene-4-sulfonyloxymethyl)-2,3-dihydro-indole

N—BOC—(S)-2-Hydroxymethyl-2,3-dihydro-indole (3.44 g, 13.8 mmol) was dissolved in CH₂Cl₂ (20 mL). Toluenesulfonyl chloride (3.42 g, 17.9 mmol) and pyridine (10 mL) were added, and the reaction was stirred at room temperature for 36 hours. The reaction mixture was diluted with CH₂Cl₂ and stirred with H₂O vigorously for 30 minutes. The aqueous layer was extracted with CH₂Cl₂, the combined organics were dried over MgSO₄, filtered, and concentrated, and the crude material was purified by silica gel chromatography (100% CH₂Cl₂) to give the desired product as art off-white solid.

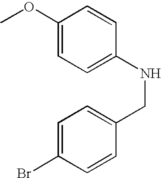

### Step 2: N-(4-Bromo-benzyl)-N-(4-methoxy-phenyl)-hydrazine hydrochloride

A solution of 4-methoxyphenylhydrazine hydrochloride (100 g) 4-bromobenzylbromide (150 g) and triethylamine (167 mL) in toluene (1.6 L) was heated to 100° C. for 3 hours. The solution was cooled and 800 mL diethyl ether was added to produce a precipitate, which was removed by filtration. The filtrate was concentrated, and the residue was dissolved in EtOAc (1 L). A saturated solution of HCl in EtOAc was added, and the precipitate was collected by filtration and dried in vacuo to give a cream solid (167 g).

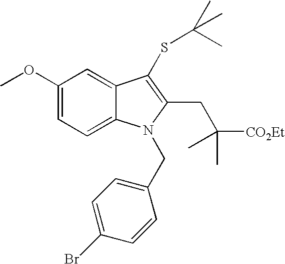

### Step 3: 3-[1-(4-Bromo-benzyl)-3-tert-butylsulfanyl-5-methoxy-1H-indol-2-yl]-2,2-dimethyl-propionic acid ethyl ester

A solution of N-(4-bromo-benzyl)-N-(4-methoxy-phenyl)-hydrazine hydrochloride (167 g), ethyl 5-(t-butylthio)-2,2-dimethyl-4-oxo-pentanoate (prepared according to the procedures described in U.S. Pat. No. 5,288,743 issued Feb. 22, 1994; 130 g), sodium acetate (80 g), and acetic acid (800 mL) in toluene (1.6 L) was stirred at room temperature for 4 days. Tire mixture was poured into H₂O and extracted with 4 times with EtOAc, then washed with brine, dried over MgSO₄, filtered and concentrated to give a dark red-black oil. Trituration with hexane:EtOAc (20:1) afforded the title compound as an off-white solid (105 g). Silica gel chromatography of the mother liquor (20% EtOAc in hexanes) afforded 16 g of the title compound, giving a combined recovery of the desired indole of 121 g.

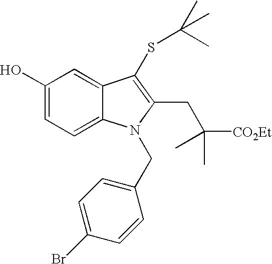

### Step 4: 3-[1-(4-Bromo-benzyl)-3-tert-butylsulfanyl-5-hydroxy-1H-indol-2-yl]-2,2-dimethyl-propionic acid ethyl ester

To 3-[1-(4-Bromo-benzyl)-3-tert-butylsulfanyl-5-methoxy-1H-indol-2-yl]-2,2-dimethyl-propionic acid ethyl ester (121 g) and 2-methyl-2-propanethiol (200 mL) in CH₂Cl₂ (400 mL) at 0° C. was added aluminum chloride (100 g) in portions over 5 minutes. After 2 hours, the mixture was carefully quenched by addition to ice and then 1N HCl (500 mL) was added. The mixture was extracted twice with CH₂Cl₂, the combined organic layers were washed with H₂O, dried over MgSO₄, filtered, and concentrated. Trituration of the residue with hexane:EtOAc (5:1) afforded the title compound as a solid (96 g).

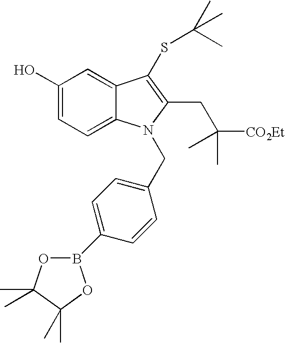

### Step 5: 3-{3-tert-Butylsulfanyl-5-hydroxy-1-[4-(4,4,5,5-tetramethyl-[1,3,2]dioxaborolan-2-yl)-benzyl]-1H-indol-2-yl}-2,2-dimethyl-propionic acid ethyl ester

3-[1-(4-Bromo-benzyl)-3-tert-butylsulfanyl-5-hydroxy-1H-indol-2-yl]-2,2-dimethyl-propionic acid ethyl ester (5.0 g, 9.6 mmol), bis(pinacolato)diboron (Combi-Blocks; 2.94 g, 11.6 mmol), and potassium acetate (2.75 g, 28.9 mmol) were dissolved in 1,4-diaxane (30 mL) in a pressure flask and degassed with N₂ for 30 minutes. Pd(dppf)Cl₂ (0.79 g, 1.0 mmol) was added, and the reaction, mixture was degassed an additional 30 minutes with N₂. The flask was then sealed and the reaction was heated at 110° C. overnight. The reaction mixture was partitioned between H₂O and EtOAc, the aqueous layer was extracted three times with EtOAc, and then the combined organic layers were washed with H₂O, brine, dried over MgSO₄, filtered, and concentrated. The crude material was purified by silica gel chromatography (0 to 50% EtOAc in hexanes) to give the desired product.

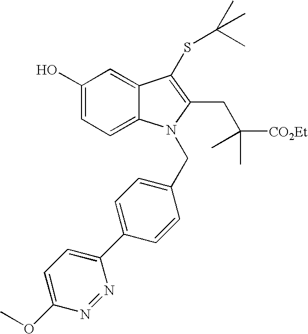

### Step 6: 3-{3-tert-Butylsulfanyl-5-hydroxy-1-[4-(6-methoxy-pyridazin-3-yl)-benzyl]-1H-indol-2-yl}-2,2-dimethyl-propionic acid ethyl ester

3-{3-tert-Butylsulfanyl-5-hydroxy-1-[4-(4,4,5,5-tetramethyl-[1,3,2]dioxaborolan-2-yl)-benzyl]-1H-indol-2-yl}-2,2-dimethyl-propionic acid ethyl ester (0.5 g, 0.8 mmol), 3-chloro-6-methoxypyridazine (Sigma-Aldrich; 0.19 g, 1.3 mmol), and potassium carbonate (0.3 g, 2.2 mmol) were dissolved in DME (6 mL) and H₂O (2 mL) and degassed with N₂ for 10 minutes. Tetrakis(triphenylphosphine)palladium(0) (0.1 g, 0.09 mmol) was added, and the reaction mixture was degassed with N₂ for an additional 15 minutes. The solution was heated to 85° C. overnight, and then cooled to room temperature and diluted with EtOAc and H₂O. The aqueous layer was extracted 3 times with EtOAc, and the combined organic layers were washed with H₂O, brine, dried over MgSO₄, filtered, and concentrated. The crude material was purified by silica gel chromatography (0 to 75% EtOAc in hexanes) to give the desired product.

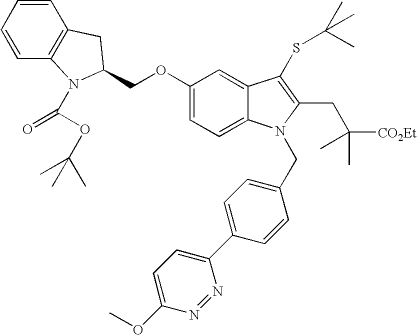

### Step 7: (S)-2-{3-tert-Butylsulfanyl-2-(2-carboxy-2-methyl-propyl)-1-[4-(6-methoxy-pyridazin-3-yl)-benzyl]-1H-indol-5-yloxymethyl}-2,3-dihydro-indole-1-carboxylic acid tert-butyl ester

To 3-{3-tert-butylsulfanyl-5-hydroxy-1-[4-(6-methoxy-pyridazin-3-yl)-benzyl]-1H-indol-2-yl}-2,2-dimethyl-propionic acid ethyl ester (0.2 g, 0.37 mmol) in DMF (2 mL) was added N—BOC—(S)-2-(toluene-4-sulfonyloxymethyl)-2,3-dihydro-indole (0.18 g, 0.45 mmol), cesium carbonate (0.24 g, 0.74 mmol), and catalytic potassium iodide. The reaction was heated to 60° C. overnight, after which LCMS analysis showed the reaction was complete. The reaction mixture was diluted with EtOAc, washed with H₂O, dried over Na₂SO₄, filtered, and concentrated. The residue was purified by silica gel chromatography (0 to 30% EtOAc in hexanes) to give the desired product.

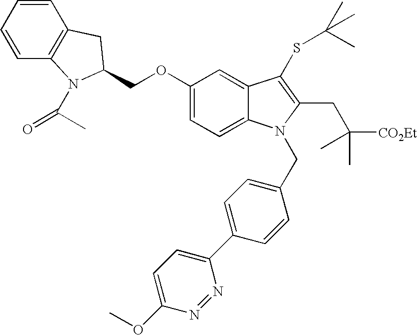

### Step 8: 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid ethyl ester

(S)-2-{3-tert-Butylsulfanyl-2-(2-carboxy-2-methyl-propyl)-1-[4-(6-methoxy-pyridazin-3-yl)-benzyl]-1H-indol-5-yloxymethyl}-2,3-dihydro-indole-1-carboxylic acid tert-butyl ester (0.23 g, 0.30 mmol) was dissolved in CH₂Cl₂ (1.5 mL). TFA (1.5 mL) was added and the reaction was stirred at room temperature for 10 minutes until no starting material was seen by LCMS analysis. The solution was concentrated in vacuo, and the crude product, 3-{3-tert-butylsulfanyl-5-[(S)-1-(2,3-dihydro-1H-indol-2-yl)methoxy]-1-[4-(6-methoxy-pyridazin-3-yl)-benzyl]-1H-indol-2-yl}-2,2-dimethyl-propionic acid ethyl ester (0.2 g, 0.30 mmol) was dissolved in CH₂Cl₂ (1 mL). Diisopropylethylamine (0.5 mL) was added, followed by acetic anhydride (0.03 mL, 0.35 mmol), and the reaction was stirred at room temperature until complete by LCMS analysis. The reaction was quenched with methanol, stirred for 10 minutes, and evaporated, to dryness under vacuum. The residue was purified y silica gel chromatography (0 to 40% EtOAc in hexanes) to give the desired product.

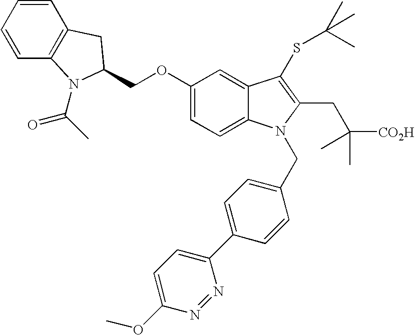

### Step 9: 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid

3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid ethyl ester (0.05 g, 0.07 mmol) was dissolved in MeOH (0.1 mL), THF (0.1 mL), and H₂O (0.1 mL). Lithium hydroxide (0.02 g, 0.58 mmol) was added, and the reaction was heated for 12 hours until no starting material was seen by tlc analysis. The reaction was diluted with H₂O, acidified to pH 5 with citric acid, and extracted with EtOAc. The combined organic layers were washed with H₂O, dried over MgSO₄, filtered, and concentrated. The residue was purified by silica gel chromatography (0 to 40% EtOAc in hexanes) to give the desired product.

### Example 4a

### Synthesis of 3-[3-tert-Butylsulfanyl-1-(4-chloro-benzyl)-5-((S)-1-isobutyryl-pyrrolidin-2-ylmethoxy)-1H-indol-2-yl]-2,2-dimethyl-propionic acid (Compound 1-4a)

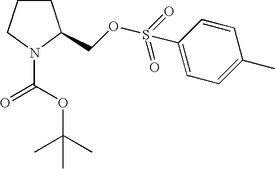

### Step 1: N—BOC—(S)-2-(toluene-4-sulfonyloxymethyl)pyrrolidine

N—BOC—(S)-2-Hydroxymethylpyrrolidine (11.0 g, 5.0 mmol) was dissolved in pyridine (3 mL), and toluenesulfonyl chloride (1.04 g, 5.5 mmol) was added. The reaction was stirred at room temperature until complete by tlc analysis, and then concentrated in vacuo. The crude material was purified y silica gel chromatography (0 to 10% EtOAc in hexanes) to give the desired product.

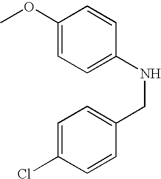

### Step 2: N-(4-Chloro-benzyl)-N-(4-methoxy-phenyl)-hydrazine Hydrochloride

A solution of 4-methoxyphenylhydrazine hydrochloride (10.0 g, 57.3 mmol), 4-chlorobenzylchloride (9.2 g, 57.2 mmol), tetrabutylammonium bromide (3.7 g, 11.5 mmol), and diisopropylethylamine (20 mL, 115 mmol) in CH₂Cl₂ (250 mL) was stirred at room temperature for several days. The reaction mixture was diluted with H₂O and the organic layer was separated, dried over MgSO₄, filtered, and concentrated. The residue was taken up in toluene (200 mL) and diethyl ether (100 mL), and 1 equivalent of 4N HCl in 1,4-dioxane was added at 0° C. The mixture was stirred at room temperature for 2 hours, and men evaporated to dryness to give the desired product as a purple solid.

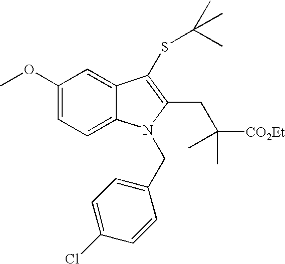

### Step 3: 3-[1-(4-Chloro-benzyl)-3-tert-butylsulfanyl-5-methoxy-1H-indol-2-yl]-2,2-dimethyl-propionic acid ethyl ester

N-(4-Chloro-benzyl)-N-(4-methoxy-phenyl)-hydrazine hydrochloride (˜16 g, 57.3 mmol), ethyl 5-(t-butylthio)-2,2-dimethyl-oxo-pentanoate (prepared according to the procedures described in U.S. Pat. No. 5,288,743 issued Feb. 22, 1994; 14.8 g, 57.3 mmol), sodium acetate (5.2 g), and acetic acid (66 mL) were combined in toluene (120 mL) and stirred at room temperature in the dark for 5 days. The mixture was partitioned between EtOAc and H₂O, and the organic layer was stirred with solid NaHCO₃, filtered, and evaporated. The residue was purified by silica gel chromatography (0 to 55% CH₂Cl₂ in hexanes), and the isolated product was recrystallized from hexanes to give the desired product.

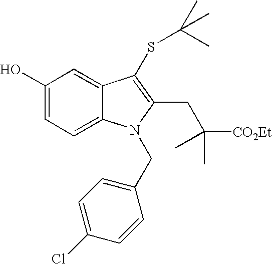

### Step 4: 3-[1-(4-Chloro-benzyl)-3-tert-butylsulfanyl-5-hydroxy-1H-indol-2-yl]-2,2-dimethyl-propionic acid ethyl ester

Aluminum chloride (0.820 g, 6.15 mmol) was suspended in 2-methyl-2-propanethiol (1.8 mL, 16 mmol) and cooled to 0° C. 3-[1-(4-Chloro-benzyl)-3-tert-butylsulfanyl-5-methoxy-1H-indol-2-yl]-2,2-dimethyl-propionic acid ethyl ester (1.0 gm 2.0 mmol) was added in CH₂Cl₂ (2.4 mL), and the reaction was allowed to warm to room temperature. After 3 hours, the reaction was complete by tlc analysis, so the solution was diluted with CH₂Cl₂ and washed with ice-cold aqueous 10% HCl. The aqueous layer was extracted three times with CH₂Cl₂, and the combined organics were dried over MgSO₄, filtered, and concentrated to give the desired product as a colourless foam.

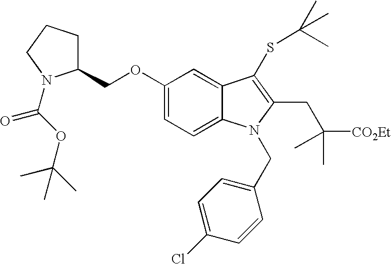

### Step 5: (S)-2-[3-tert-Butylsulfanyl-1-(4-chloro-benzyl)-2-(2-ethoxycarbonyl-2-methyl-propyl)-1H-indol-5-yloxymethyl]-pyrrolidine-1-carboxylic acid tert-butyl ester

To 3-[1-(4-chloro-benzyl)-3-tert-butylsulfanyl-5-hydroxy-1H-indol-2-yl]-2,2-dimethyl-propionic acid ethyl ester (0.5 g, 1.05 mmol) in DMF (2.5 mL) was added N—BOC—(S)-2-(toluene-4-sulfonyloxymethyl)pyrrolidine (0.39 g, 1.10 mmol) and cesium carbonate (0.69 g, 2.1 mmol). The reaction was stirred at 45° C. for 2 hours, and then catalytic potassium iodide was added and the reaction was heated to 60° C. overnight. The reaction mixture was diluted with EtOAc, washed with H2O, dried over Na₂SO₄, filtered, and concentrated. The residue was purified by silica gel chromatography (0 to 15% EtOAc in hexanes) to give the desired product.

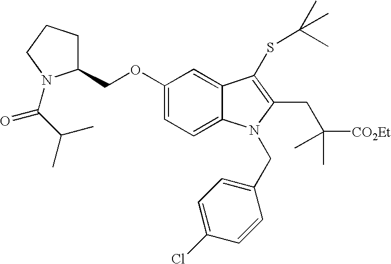

### Step 6: 3-[3-tert-Butylsulfanyl-1-(4-chloro-benzl)-5-((S)-1-isobutyryl-pyrrolidine-2-ylmethoxy)-1-H-indol-2-yl]-2,2-dimethyl-propionic acid ethyl ester

(S)-2-[3-tert-Butylsulfanyl-1-(4-chloro-benzyl)-2-(2-ethoxycarbonyl-2-methyl-propyl)-1H-indol-5-yloxymethyl]-pyrrolidine-1-carboxylic acid tert-butyl ester (0.94 g, 1.43 mmol) was dissolved in CH₂Cl₂ (5 mL). TFA (2 mL) was added and the reaction was stirred at room temperature for 4 hours until no starting material was seen by tlc analysis. The solution was concentrated in vacuo, and the crude product, 3-[3-tert-butylsulfanyl-1-(4-chloro-benzyl)-5-((S)-pyrrolidin-2-ylmethoxy)-1H-indol-2-yl]-2,2-dimethyl-propionic acid ethyl ester (0.2 g, 0.31 mmol) was dissolved in CH₂Cl₂ (3 mL). Diisopropylethylamine (1 mL) was added, followed by isobutyryl chloride (0.36 mL, 0.34 mmol), and the reaction was stirred at room temperature for 1 hour. The reaction was quenched with methanol, stirred for 10 minutes, and evaporated to dryness under vacuum. The residue was purified by silica gel chromatography (0 to 40% EtOAc in hexanes) to give the desired product.

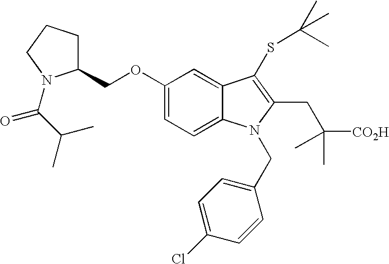

### Step 7: 3-[3-tert-Butylsulfanyl-1-(4-chloro-benzyl)-5-((S)-1-isobutyryl-pyrrolidin-2-ylmethoxy)-1H-indol-2-yl]-2,2-dimethyl-propionic acid

3-[3-tert-Butylsulfanyl-1-(4-chloro-benzyl)-5-((S)-1-isobutyryl-pyrrolidin-2-ylmethoxy)-1H-indol-2-yl]-2,2-dimethyl-propionic acid ethyl ester (0.16 g, 0.26 mmol) was dissolved in MeOH (1 mL), THF (1 mL), and H₂O (1 mL), Lithium hydroxide (0.6 g, 1.43 mmol) was added, and the reaction was heated for 12 hours until no starting material was seen by tlc analysis. The reaction was diluted with H₂O, acidified to pH 5 with citric acid, and extracted with EtOAc. The combined organic layers were washed with H₂O, dried over MgSO₄, filtered, and concentrated. The residue was purified on silica gel (0 to 40% EtOAc in hexanes) to give the desired product.

### Example 5a

### Synthesis of 3-[3-tert-Butylsulfanyl-1-[4-(5-hydroxymethyl-pyridin-2-yl)-benzyl]-5-(5-methyl-pyridin-2-ylmethoxy)-1H-indol-2-yl]-2,2-dimethyl-propionic acid (Compound 1-5a)

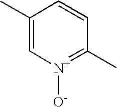

### Step 1: 2,5-Dimethyl-pyridine 1-oxide

2,5-Lutidine (5.0 g, 46.7 mmol) was dissolved in CHCl₃ (125 mL) and cooled to 0° C. m-Chloroperoxybenzoic acid (70%; 13.9 g, 55.2 mmol) was added, and the reaction was stirred overnight at room temperature. The mixture was washed with saturated aqueous Na₂CO₃, dried over Na₂SO₄, filtered, and concentrated to give the desired product (5.7 g).

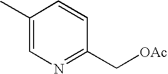

### Step 2: Acetic acid 5-methyl-pyridin-2-ylmethyl ester

2,5-Dimethyl-pyridine 1-oxide (5.7 g, 46.7 mmol) was dissolved in acetic anhydride (25 mL) and heated to reflux at 100° C. for one hour. The mixture was cooled to room temperature, and ethanol (2.7 mL, 46.7 mmol) was slowly added to quench the reaction. The solution was evaporated to dryness and purified by silica gel chromatography to give the desired product (7.7 g).

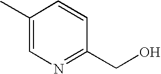

### Step 3: (5-Methyl-pyridin-2-yl)-methanol

Acetic acid 5-methyl-pyridin-2-ylmethyl ester (7.7 g, 46.7 mmol) was dissolved in concentrated HCl (20 mL) and refluxed for 1 hour. The reaction was cooled and evaporated to dryness to give an orange solid, which was used directly in the next reaction.

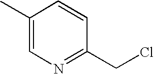

### Step 4: 2-Chloromethyl-5-methyl-pyridine

(5-Methyl-pyridin-2-yl)-methanol (1.0 g, 8.1 mmol) was dissolved in thionyl chloride (3 mL) and stirred at room temperature for 30 minutes under N₂. The mixture was evaporated to dryness to give the desired product as a hydrochloride salt, which was used as obtained in subsequent reactions.

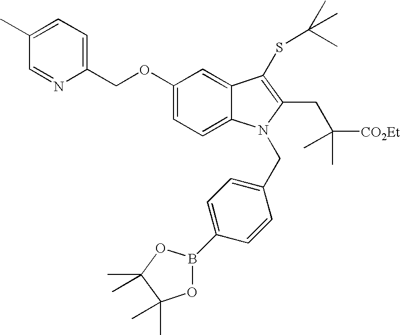

### Step 5: 3-{3-tert-Butylsulfanyl-5-(5-methyl-pyridin-2-ylmethoxy)-1-[4-(4,4,5,5-tetramethyl-[1,3,2]dioxaborolan-2-yl)-benzyl]-1H-indol-2-yl}-2,2-dimethyl-propionic acid ethyl ester

3-{3-tert-Butylsulfanyl-5-hydroxy-1-[4-(4,4,5,5-tetramethyl-[1,3,2]dioxaborolan-2-yl)-benzyl]-1H-indol-2-yl}-2,2-dimethyl-propionic acid ethyl ester (4.53 g, 8.0 mmol), 2-chloromethyl-5-methyl-pyridine (1.71 g, 9.6 mmol), cesium carbonate (7.80 g, 24.0 mmol), and tetrabutylammonium bromide (0.13 g, 0.4 mmol) were combined in 3:1 MeCN:DMF (100 mL). The reaction was heated to 90° C. for 4 hours, and then 70° C. overnight. LCMS analysis showed 15% starting material remained, so additional 2-chloromethyl-5-methy)-pyridine (0.5 g) and cesium carbonate (1.5 g) were added, and the temperature was increased to 105° C. for 2 hours. Once no starting material was seen by LCMS analysts, the mixture was diluted with H₂O and extracted with EtOAc. The combined organic layers were dried over MgSO₄, filtered, and concentrated, and the residue was purified by silica gel chromatography to give the desired product (3.78 g).

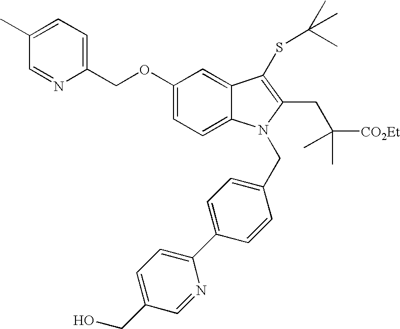

### Step 6; 3-[3-tert-Butylsulfanyl-1-[4-(5-hydroxymethyl-pyridin-2-yl)-benzyl]-5-(5-methyl-pyridin-2-ylmethoxy)-1H-indol-2-yl]-2,2-dimethyl-propionic acid ethyl ester

3-{3-tert-Butylsulfanyl-5-(5-methyl-pyridin-2-ylmethoxy)-1-[4-(4,4,5,5-tetramethyl-[1,3,2]dioxaborolan-2-yl)-benzyl]-1H-indol-2-yl}-2,2-dimethyl-proprionic acid ethyl ester (3.78 g, 5.63 mmol), 2-chloro-5-hydroxymethylpyridine (0.973 g, 6.76 mmol), and potassium carbonate (2.72 g, 19.7 mmol) were combined in 2:1. DME:H₂O (60 mL) and degassed with N₂ for 10 minutes. Tetrakis(triphenylphosphine)palladium(0) (0.325 g, 0.28 mmol) was added, and the reaction was stirred at 80° C. overnight. Once no starting material was seen by analytical LCMS, the mixture was diluted with H₂O and extracted with EtOAc, and the combined organic layers were dried over MgSG₄, filtered, and concentrated. The residue was purified by silica gel chromatography to give the desired product (2.96 g).

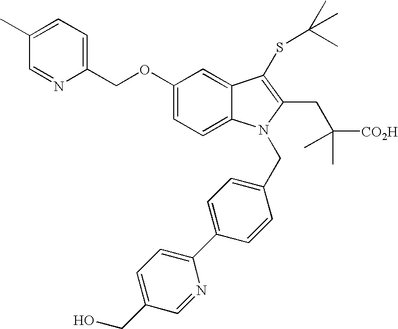

### Step 7: 3-[3-tert-Butylsulfanyl-1-[4-(5-hydroxymethyl-pyridin-2-yl)-benzyl]-5-(5-methyl-pyridin-2-ylmethoxy)-1H-indol-2-yl]-2,2-dimethyl-propionic acid

To 3-[3-tert-butylsulfanyl-1-[4-(5-hydroxymethyl-pyridin-2-yl)-benzyl]-5-(5-methyl-pyridin-2-ylmethoxy)-1H-indol-2-yl]-2,2-dimethyl-propionic acid ethyl ester (0.415 g, 0.64 mmol) in 1:1 MeOH:THF (12 mL) was added aqueous LiOH (1N; 3.0 mL), and the reaction mixture was stirred, for 2 hours at 65° C. The solution was neutralized to pH 5-6 and extracted with EtOAc. The combined organic layers were dried over MgSO₄, filtered, and concentrated to give the desired product (0.420 g).

### Example 6a

### Synthesis of 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2,3-dihydroxy-propyl ester (Compound 1-6a)

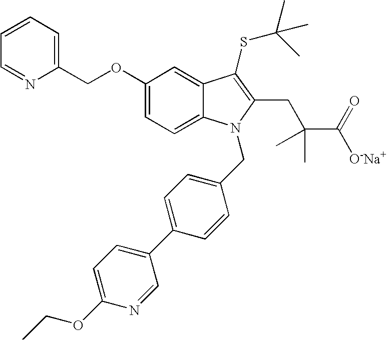

### Step 1: 3-[3-tert-Butylsulfanyl-1-[4-(6-ethoxy-pyridin-3-yl)-benzyl]-5-(pyridin-2-yl]-2,2-dimethyl-propionic acid sodium salt

To 3-[3-tert-Butylsulfanyl-1-[4-(6-ethoxy-pyridin-3-yl)-benzyl]-5-(pyridin-2-yl]-2,2-dimethyl-propionic acid (prepared according to the procedures described in US patent publication no. 2007/0105866; see Compound 2-94; 0.067 g, 0.107 mmol) in EtOH. (3 mL) was added 1N aqueous NaOH (0.11 mL, 0.109 mmol), and the solution was stirred at room temperature for 15 minutes. The mixture was then concentrated to dryness, and the residue was dissolved in deionized H₂O and frozen. The H₂O was removed by lyophilization to give the desired salt as a white powder.

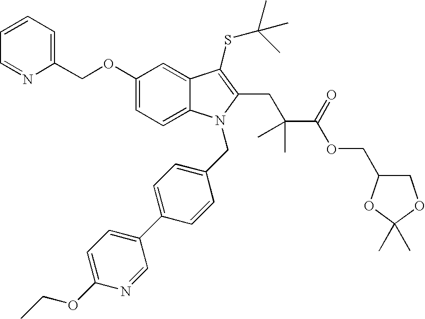

### Step 2: 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2,2-dimethyl-[1,3]dioxolan-4-ylmethyl ester

3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid sodium salt (0.500 g, 0.77 mmol) was dissolved in dichloroethane (3 mL) and cooled to 0° C. Oxalyl chloride (0.08 mL, 0.92 mmol) was added, and the reaction was stirred for 15 minutes. DL-1,2-Isopropylideneglycerol (0.163 g, 1.23 mmol), diisopropylethylamine (0.40 mL, 2.30 mmol), and catalytic DMAP were then added, and the mixture was stirred at room temperature overnight. Once no starting material was seen by analytical LCMS, the solution was partitioned between EtOAc and H₂O, and the aqueous layer was extracted with EtOAc. The combined organic layers were dried over MgSO₄, filtered, and concentrated, and the residue was purified by silica gel chromatography to give the desired product.

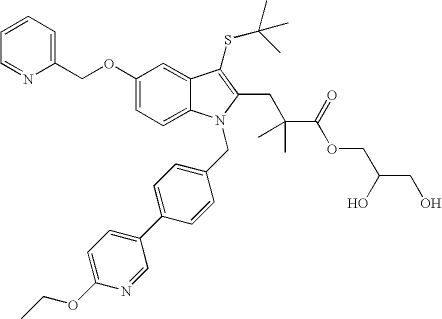

### Step 3: 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2,3-dihydroxy-propyl ester

To 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2,2-dimethyl-[1,3]dioxolan-4-ylmethyl ester (0.080 g, 0.11 mmol) in THF (2 mL) and H₂O (1 mL) was added HCl (4N in 1,4-dioxane; 0.5 mL), and the reaction was stirred at room temperature until no starting material was seen by LCMS analysis. The mixture was quenched with saturated aqueous NaHCO₃ and extracted twice with EtOAc. The combined organic layers were dried over MgSO₄, filtered, and concentrated, and the residue was purified by silica gel chromatography to give the desired product.

### Example 7a Synthesis of 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid isosorbide ester (Compound 1-7a)

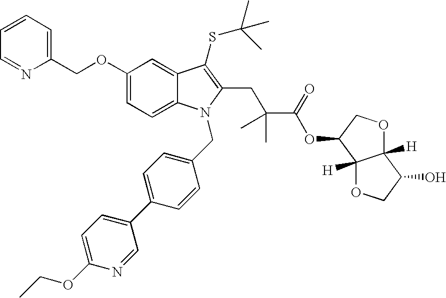

### Step 1: 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid isosorbide ester

3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid sodium salt (0.500 g, 0.7 mmol) was dissolved in dichloroethane (3 mL) and cooled to 0° C. Oxalyl chloride (0.08 mL, 0.92 mmol) was added, and the reaction was stirred for 15 minutes. Isosorbide (0.169 g, 1.55 mmol), diisopropylethylamine (0.50 mL, 2.86 mmol), and catalytic DMAP were then added, and the mixture was stirred at room temperature overnight. Once no starting material was seen by analytical LCMS, the solution was partitioned between EtOAc and H₂O, and the aqueous layer was extracted with EtOAc. The combined organic layers were dried over MgSO₄, filtered, and concentrated, and the residue was purified by silica gel chromatography to give the desired product.

### Preparation of NO-Releasing Compounds

In one aspect, the synthesis of NO-releasing compounds is performed as described in the following scheme.

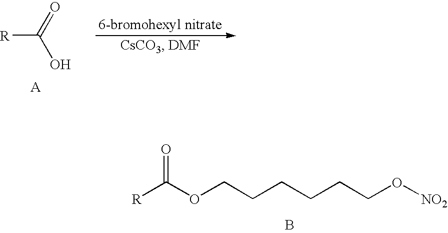

### General Procedure:

To a mixture of acid A (1.26 mmol) and 6-bromohexyl nitrate (F. Conrad Engelhardt et al, J. Org. Chem., 2006, 71, 480-491; 1.4 mmol) in DMF (5 ml) was added Cs₂CO₃ (6 mmol). The mixture was stirred at room temperature overnight. Water was added and the reaction mixture was extracted with ethyl acetate. The extracts were washed with brine, dried over Na₂SO₄, filtered, and the solvent was evaporated under reduced pressure. The residue obtained was purified by chromatography (silica gel eliding with 3:1 Hex:EtOAc) to give product B.

### Example 1b

### Synthesis of 3-[3-tert-Butylsulfanyl-1-(4-chloro-benzyl)-5-isopropyl-1H-indol-2-yl]-2,2-dimethyl-propionic acid 6-nitrooxy-hexyl ester (Compound 1-1b)

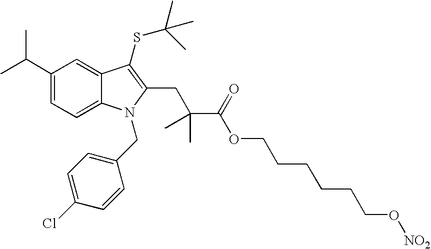

### Step 1: 3-[3-tert-Butylsulfanyl-1-(4-chloro-benzyl)-5-isopropyl-1H-indol-2-yl]-2,2-dimethyl-propionic acid 6-nitrooxy-hexyl ester

To a mixture of 3-[3-tert-butylsulfanyl-1-(4-chloro-benzyl)-5-isopropyl-1H-indoi-2-yl]-2,2-dimethyl-propionic acid (1.26 mmol) and 6-bromohexyl nitrate (F. Conrad Engelhardt et al, J. Org. Chem., 2006, 71, 480-491; 1.4 mmol) in DMF (5 ml) was added CsCO₃ (6 mmol). The mixture was stirred at room temperature over night. H₂O was added and the reaction mixture was extracted with ethyl acetate. The extracts were washed with brine, dried over Na₂SO₄, filtered, and the solvent was evaporated under reduced pressure. The residue obtained was purified by silica gel chromatography (25% EtOAc in hexanes) to give the desired product.

¹H NMR δ 7.63 (s, 1H), 7.39 (d, 2H), 7.02 (s, 2H), 6.74 (d, 2H), 5.32 (s, 2H), 4.38 (t, 2H), 3.97 (t, 2H), 3.25 (s, 2H), 3.01 (m, 1H), 1.62-1.15 (m, 29H).

### Example 2b

### Synthesis of 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid 6-nitrooxy-hexyl ester (Compound 1-2b)

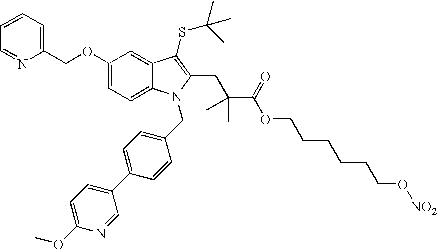

### Step 1: 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid 6-nitrooxy-hexyl ester

Prepared according to the procedure described in Example 1b, Step 1, using the folio wing starting materials: 3-[3-tert-Butylsulfanyl-1-[4-(6-methoxy-pyridin-3-yl)-benzyl]-5-(pyridin-2-ylmethoxy)-1H-indol-2-yl]-2,2-dimethyl-propionic acid and 6-bromohexyl nitrate.

¹H NMR (CDCl₃) δ 8.6 (d, 1H), 8.31 (d, 1H), 7.70 (dd, 2H), 7.57 (d, 1H), 7.38 (d, 2H), 7.32 (d, 1H), 7.20 (m, 1H), 7.08 (d, 1H), 6.89 (dd, 1H), 6.84 (d, 2H), 6.77 (d, 1H), 5.41 (s, 2H), 5.27 (s, 2H), 4.36 (t, 2H), 3.96 (m, 5H), 3.28 (s, 2H), 1.57-1.20 (m, 23H).

### Example 3b

### Synthesis of 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid 6-nitrooxy-hexyl ester (Compound 1-3b)

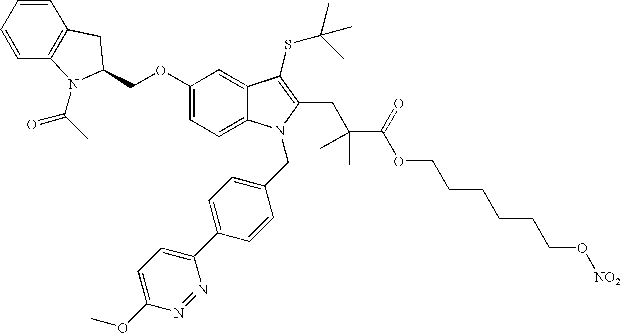

### Step 1: 3-{5-((S)-1-Acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid 6-nitrooxy-hexyl ester

Prepared according to the procedure described in Example 1b, Step 1, using the following starting materials: 3-{5-((S)-1-acetyl-2,3-dihydro-1H-indol-2-ylmethoxy)-3-tert-butylsulfanyl-1-[4-(6-methoxy-pyridazin-3-yl)-benzyl]-1H-indol-2-yl}-2,2-dimethyl-propionic acid and 6-bromohexyl nitrate.

¹H NMR (CDCl₃) δ 7.86 (d, 2H), 7.70 (d, 1H), 7.24 (d, 1H), 7.21 (dd, 4H), 6.87 (d, 2H), 6.73 (d, 1H), 5.42 (s, 2H), 4.34 (t, 2H), 4.23 (s, 3H), 3.97 (t, 2H), 3.3 (m, 4H), 1.62-1.20 (m, 23H).

### Example 4b

### Synthesis of 3-[3-tert-Butylsulfanyl-1-(4-chloro-benzyl)-5-((S)-1-isobutyryl-pyrrolidin-2-ylmethoxy)-1H-indol-2-yl]-2,2-dimethyl-propionic acid 6-nitroxy-hexyl ester (Compound 1-4b)

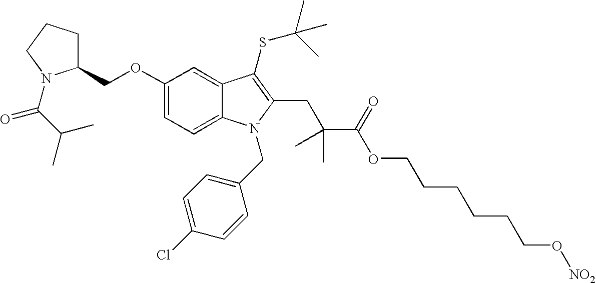

### Step 1: 3-[3-tert-Butylsulfanyl-1-(4-chloro-benzyl)-5-((S)-1-isobutyryl-pyrrolidin-2-ylmethoxy)-1H-indol-2-yl]-2,2-dimethyl-propionic acid 6-nitroxy-hexyl ester

Prepared according to the procedure described in Example 1b. Step 1, using the following starting materials: 3-[3-tert-butylsulfanyl-1-(4-chloro-benzyl)-5-((S)-1-isobutyryl-pyrrolidin-2-ylmethoxy)-1H-indol-2-yl]-2,2-dimethyl-propionic acid and 6-bromohexyl nitrate.

¹H NMR (CDCl₃) δ 8.12-8.07 (m, 1H), 7.29-7.24 (m, 2H), 7.23-7.36 (m, 2H), 7.04-6.95 (m, 1H), 6.86-6.75 (m, 1H), 6.74-6.67 (m, 2H), 5.38-5.29 (m, 2H), 4.72-4.67 (m, 1H), 4.52-4.43 (m, 1H), 4.42-4.36 (m, 2H), 4.22-4.01 (m, 2H), 4.00-3.72 (m, 2H), 3.71-3.46 (m, 3H), 3.47-3.35 (m, 1H), 3.30-3.18 (m, 2H), 2.23-1.84 (m, 3H), 1.69-1.44 (m, 6H), 1.35-1.17 (m, 15H), 1.17-1.18 (m, 3H), 0.94-0.82 (m, 2H).

### Example 5b

### Synthesis of 3-{3-tert-Butylsulfanyl-5(5-methyl-pyridin-2-ylmethoxy)-1-[4-(5-nitrooxymethyl-pyridin-2-yl)-benzyl]-1H-indol-2-yl}-2,2-dimethyl-propionic acid (Compound 1-5b)

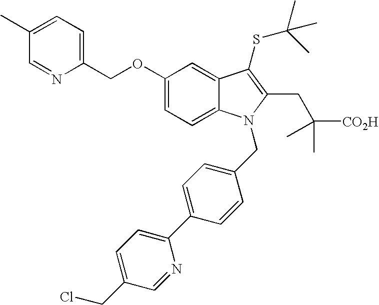

### Step 4: 3-[3-tert-Butylsulfanyl-1-[4-(5-chloromethyl-pyridin-2-yl)-benzyl]-5-(5-methyl-pyridin-2-ylmethoxy)-1H-indol-2-yl]-2,2-dimethyl-propionic acid

To 3-[3-tert-Butylsulfanyl-1-[4-(5-chloromethyl-pyridin-2-yl)-benzyl]-5-(5-methyl-pyridin-2-ylmethoxy)-1H-indol-2-yl]-2,2-dimethyl-propionic acid (0.195 g, 0.31 mmol) in CH₂Cl₂ (10 mL) was added thionyl chloride (0.45 mL, 6.2 mmol), and the reaction was stirred for 1 hour at room temperature. The mixture was concentrated and then dissolved in THF (20 mL) and treated with aqueous LiOH (1N; 4.0 mL). After stirring vigorously for 20 minutes, the reaction was quenched with aqueous HCl (1N, 5 mL) and partitioned between EtOAc (100 mL) and H₂O (20 mL). The organic layer was washed with H₂O and brine, dried over MgSO₄, filtered, and concentrated to the desired product (0.175 g).

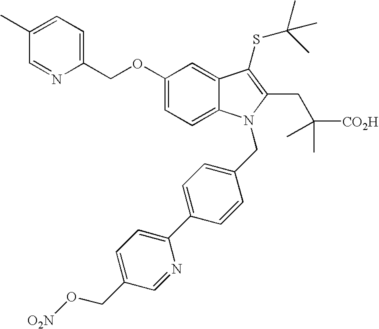

### Step 5: 3-{3-tert-Butylsulfanyl-5-(5-methyl-pyridin-2-ylmethoxy)-1-[4-(5-nitrooxymethyl-pyridin-2-yl)-benzyl]-1H-indol-2-yl}-2,2-dimethyl-propionic acid

3-[3-tert-Butylsulfanyl-1-[4-(5-chloromethyl-pyridin-2-yl)-benzyl]-5-(5-methyl-pyridin-2-ylmethoxy)-1H-indol-2-yl]-2,2-dimethyl-propionic acid (0.173 g, 0.27 mmol) was dissolved in MeCN (5 mL) at room temperature. Silver nitrate (0.064 g, 0.38 mmol) and tetrabutylammonium bromide (0.010 g, 0.03 mmol) were added, and the reaction was covered with foil and heated to 80° C. for 5 hours. Once no starting material was seen by analytical LCMS, the mixture was partitioned between EtOAc and H₂O. The aqueous layer was extracted with EtOAc, and the combined organic layers were dried over MgSO₄, filtered, and concentrated. The residue was purified by preparative HPLC to give the desired product (0.048 g).

### Example 6b

### Synthesis of 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2-hydroxy-3-nitrooxy-propyl ester (Compound 1-6b)

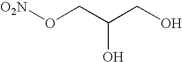

### Step 1: 3-Nitrooxy-propane-1,2-diol

To glycerol (0.92 g, 10.0 mmol) in THF (23 mL) was added silver nitrate (1.70 g, 10.0 mmol), followed by thionyl chloride (0.73 mL, 10.0 mmol) dropwise. The solution was stirred overnight at room temperature, with a precipitate slowly appearing during tire course of the reaction. The mixture was partitioned between EtOAc and H₂O, and the aqueous layer was extracted with EtOAc. The combined organic layers were dried over MgSO₄, filtered, and concentrated, and the residue was purified by silica gel chromatography (0-50% EtOAc in hexanes) to give the desired product.

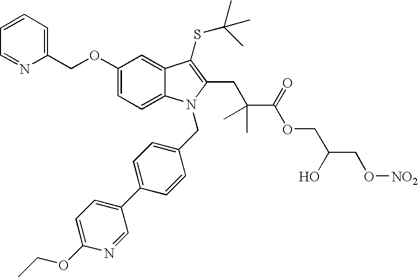

### Step 2: 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid 2-hydroxy-3-nitrooxy-propyl ester

3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid sodium salt (0.400 g, 0.62 mmol) was dissolved in dichloroethane (3 mL) and cooled to 0° C. Oxalyl chloride (0.06 mL, 0.69 mmol) was added, and the reaction was stirred for 15 minutes. 3-Nitrooxy-propane-1,2-diol (0.169 g, 1.23 mmol), diisopropylethylamine (0.40 mL, 2.30 mmol), and catalytic DMAP were then added, and the mixture was stirred at room temperature overnight. Once no starting material was seen by analytical LCMS, the solution was partitioned between EtOAc and H₂O, and the aqueous layer was extracted with EtOAc. The combined organic layers were dried over MgSO₄, filtered, and concentrated, and the residue was purified by silica gel chromatography to give the desired product.

### Example 7b

### Synthesis of 3-[3-tert-Butylsulfanyl-1-[4(6-ethoxy-pyridin-3-yl)-benzyl]-5-(pyridin-2-ylmethoxy)-1H-indol-2-yl]-2,2-dimethylpropionic acid isosorbide-5-mononitrate ester (Compound 1-7b)

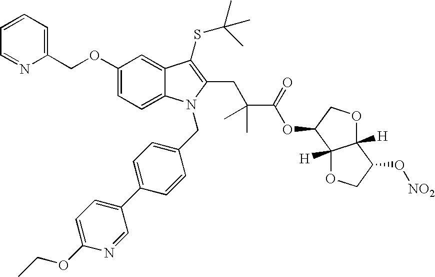

### Step 1: 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid isosorbide-5-mononitrate ester

3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid sodium salt (0.500 g, 0.77 mmol) was dissolved in dichloroethane (3 mL) and cooled to 0° C. Diisopropylethylamine (0.30 mL, 1.72 mmol) was added, followed by oxalyl chloride (0.08 mL, 0.92 mmol), and the reaction was stirred for 1.5 minutes. Isosorbide-5-mononitrate (0.250 g, 1.31 mmol) and catalytic DMAP were then added, and the mixture was stirred at room temperature overnight. Once no starting material was seen by analytical LCMS, the solution, was partitioned between EtOAc and H₂O, and the aqueous layer was extracted with EtOAc. The combined organic layers were dried over MgSO₄, filtered, and concentrated, and the residue was purified by silica gel chromatography (0-3% MeOH in CH₂Cl₂) to give the desired product.

### Example 2

### FLAP Binding Assays

A non-limiting example of such a FLAP binding assay is as follows:

Packed human polymorphonuclear cell pellets (1.8×109 cells) (Biological Specialty Corporation) were resuspended, lysed and 100,000 g membranes prepared as described (Charleson et al. Mol. Pharmacol, 41, 873-879, 1992). 100,000×g pelleted membranes were resuspended in Tris-Tween assay buffer (100 mM Tris HCl pH 7.4, 140 mM NaCl, 2 mM EDTA, 0.5 mM DTT, 5% glycerol, 0.05% Tween 20) to yield a protein concentration of 50-100 ug/mL. 10 uL membrane suspension was added to 96 well Millipore plate, 78 uL Tris-Tween buffer, 10 μL ³H MK886 or ³H 3-[5-(pyrid-2-ylmethoxy)-3-tert-butylthio-1-benzyl-indol-2-yl]-2,2-dimethylpropionic acid (or ¹²⁵I MK591 derivative Eggler et al, J. Labelled Compounds and Radiopharmaceuticals, 1994, vXXXIV, 1147)) to −30,000 cpm, 2 μL inhibitor and incubated for 30 minutes at room temperature. 100 μL ice-cold washed buffer was added to the incubation mixture. Plates were then filtered and washed 3× with 200 μL ice cold Tris-Tween buffer, scintillation bottoms sealed, 100 μL scintillant added, shaken for 15 minutes then counted in a TopCount. Specific binding was determined as defined as total radioactive binding minus non-specific binding in the presence of 10 μM MK886. IC50s were determined using Graphpad prism analysis of drug titration curves.

### Example 3

### Human Leukocyte Inhibition Assay

A non-limiting example of a human leukocyte inhibition assay is as follows:

Blood was drawn from consenting human volunteers into heparinized tubes and 3% dextran, 0.9% saline equal volume added. After sedimentation of red blood cells a hypotonic lysis of remaining red blood cells was performed and leukocytes sedimented at 1000 rpm. The pellet was resuspended at 1.25×10⁵ cells/mL and aliquoted into wells containing 2.5 μL 20% DMSO (vehicle) or 2.5 μL drug in 20% DMSO. Samples were incubated for 5 minutes at 37° C. and 2 μL calcium ionophore A23817 (from a 50 mM DMSO stock diluted just prior to the assay in Hanks balanced salt solution (Invitrogen)) to 1.25 mM) was added, solutions mixed and incubated for 30 minutes at 37° C. Samples were centrifuged at 1,000 rpm (˜200×g) for 10 minutes at 4° C., plasma removed and a 1:4 dilution assayed for LTB₄ concentration using ELISA (Assay Designs). Drug concentrations to achieve 50% inhibition (IC50's) of vehicle LTB₄ were determined by nonlinear regression (Graphpad Prism) of % inhibition versus log drug concentration. The compounds presented in Tables 1-4 had assays of 1 nM to 5 μM with this assay. Compounds presented in Example 1 that have been tested with this assay have an activity less than about 2 micromolar.

### Example 4

### Human Blood LTB₄ Inhibition Assay

A non-limiting example of such a human blood LTB₄ inhibition assay is as follows:

Blood was drawn from consenting human volunteers into heparinized tabes and 125 μL aliquots added to wells containing 2.5 μL 50% DMSO (vehicle) or 2.5 μL drug in 50% DMSO. Samples were incubated for 15 minutes at 37° C. 2 μL calcium ionophore A23817 (from a 50 mM DMSO stock diluted just prior to the assay in Hanks balanced salt solution (Invitrogen)) to 1.25 mM) was added, solutions mixed and incubated for 30 minutes at 37° C. Samples were centrifuged at 1,000 rpm (˜200×g) for 10 minutes at 4° C., plasma removed and a 1:100 dilution assayed for LTB₄ concentration using ELISA (Assay Designs). Drug concentrations to achieve 50% inhibition (IC₅₀'s) of vehicle LTB₄ were determined by nonlinear regression (Graphpad Prism) of % inhibition versus log drug concentration.

### Example 5

### Rat Peritoneal Inflammation and Edema Assay

A non-limiting example of such a rat peritoneal inflammation and edema assay is as follows:

The in vivo efficacy of leukotriene biosynthesis inhibitors was assessed using a rat model of peritoneal inflammation. Male Sprague-Dawley rats (weighing 200-300 grants) received a single intraperitoneal (i.p.) injection of 3 mL saline containing zymosan (5 mg/mL) followed immediately by an intravenous (i.v.) injection of Evans blue dye (2 mL of 1.5% solution). Compounds were administered orally (3 mL/kg in 0.5% methylcellulose vehicle) 2 to 4 hours prior to zymosan injection. One to two hours after zymosan injection, rats were euthanized, and the peritoneal cavity was flushed with 10 mL phosphate buffered saline solution (PBS). The resulting fluid was centrifuged at 1,200 rpm for 10 minutes. Vascular edema was assesses by quantifying the amount of Evans blue dye in the supernatant using a spectrophotometer (Absorbance 610 nm). LTB₄ and cysteinyl leukotriene concentrations in the supernatant were determined by ELISA. Drug concentrations to achieve 50% inhibition of plasma leakage (Evans blue dye) and inhibition, of peritoneal LTB₄ and cysteinyl leukotrienes could be calculated by nonlinear regression (Graphpad Prism) of % inhibition versus log drug concentration.

### Example 6

### NO Release: Non-Invasive Measurement of Blood Pressure

A non-limiting example of an assay for NO release is as follows:

Reduction in blood pressure is assessed in a rat hypertension model using a computerized, non-invasive tail-cuff acquisition system (model XBP1000; Kent Scientific Corporation). Prior to blood pressure recording sessions, male Sprague-Dawley rats (weighing 200-300 grams) are habituated to a standard rodent holder (XBP-RM, Kent Scientific) for 3 consecutive days (1 hour sessions). Following this habituation phase, hypertension is induced using an osmotic mini-pump (Alzet model 2001) containing Val⁵-angiotensin II (Sigma) infused at a rate of 0.7 mg/kg/day for up to 7 days (Ishizaka et ah, 1999, FEBS Letters). On day 0, 5 and 7 of infusion, animals are placed back in their holders and are fitted with an inflatable tail-cuff which measured systolic pressure, diastolic pressure and heart pulse rate. On infusion day 7, following baseline recordings, compound is administered orally (3 ml/kg in 0.5% methylcellulose vehicle) and assessed for its ability to reduce blood pressure. Drag concentrations to achieve 50% inhibition, in systolic blood pressure can be calculated by nonlinear regression (Graph pad Prism) of % inhibition versus log drug concentration.

### Example 7

### Rat Gastric Lesion Procedure

Gastric lesions in rat can be induced by ingestion of a non-steroidal anti-inflammatory drug (NSAID) such as naproxen. This effect is apparent with 4 to 6 hours of administration. The therapeutic effect of two different agents on preventing NSAID-induced gastric lesions first as a mono-therapy and then as a combination therapy using a fractional dose approach to test for additivity. The two agents tested include 1) 3-[3-tert-Butylsulfanyl-1-[4-(6-ethoxy-pyridin-3-yl)-benzyl]-5-(pyridin-2-ylmethoxy)-1H-indol-2-yl]-2,2-dimethyl-propionic acid (prepared according to the procedures described in US patent publication no. 2007/0105866; see Compound 2-94), a 5-lipoxygenase activating protein (FLAP) inhibitor and 2) isosorbide mononitrate (a nitric oxide donor).

Procedures: Gastric lesions were induced in 24-hour fasted male Sprague-Dawley rats (weighing 200-300 grams) by administering a single oral dose of naproxen (40 mg/kg), in the mono-therapy phase of the study Compound 2-94 was prepared at 1, 10 and 30 mg/kg and was co-dosed with naproxen. Separately, isosorbide mononitrate was prepared at 3, 10, 30 and 100 mg/kg and was co-dosed with naproxen. The ED50 (defined as the dose to produce the half maximal effect) from this phase of the experiment was used to calculate doses for the combination therapy phase of the study in which Compound 2-94 and isosorbide mononitrate were formulated together and co-dosed with Naproxen. Fractional doses tested include; 2, 1, ½ and ¼ of the ED50 values. In all conditions, four hours alter dosing, rats were anesthetized with inhaled isoflurane and the stomachs removed and cut along the lesser curvature and rinsed thoroughly of any contents using tap water. Each stomach was then pinned down flat with the mucosal layer side up and a digit image captured at an exposure of 10×. These images were then used to determine the total lesion area in mm². A Lesion Index (LI) was calculated for each individual stomach by the formula:

LI=[lesion area (mm²)/total stomach area (mm²)]×100

Individual drug effects were then determined by calculating the percent inhibition of LI for each dose group relative to the naproxen-alone group using the formula:

% inhibition=100−[LIₜᵣₑₐₜₘₑₙₜ/LI_{control(naproxen)}×100]

Results: Compound 2-94 caused a dose dependent inhibition of naproxen induced gastric lesions with an estimated ED50 value of 5 mg/kg. Isosorbide caused a dose dependent inhibition of naproxen induced gastric lesions with an estimated ED50 value of 15 mg/kg. From these values we chose the following fractional doses: 2×ED50 (30 mg/kg Compound 2-94+30 mg/kg isosorbide mononitrate), 1×ED50 (5 mg/kg Compound 2-94+15 mg/kg isosorbide mononitrate), ½ ED50 (2.5 mg/kg Compound 2-94+7.5 mg/kg isosorbide mononitrate) and ¼ ED50 (1.25 mg/kg Compound 2-94+3.75 mg/kg isosorbide mononitrate). The resulting fraction dose ED50 was determined to be the dose combination of 2.5 mg/kg Compound 2-94+7.5 mg/kg isosorbide mononitrate. The total fractional value was 1 using the following formula:

(ED₅₀ of Compound 2-94 with isosorbide mononitrate)/(ED₅₀ ft for Compound 2-94 given alone)+(ED₅₀ of isosorbide mononitrate with Compound 2-94)/(ED₅₀ for isosorbide given alone).

The fractional value indicates what proportion of the single ED50 value was accounted for by the corresponding ED50 value of the combination. Values near 1 indicate an additive interaction, values >1 indicate an antagonistic interaction, and values <1 indicate a synergistic interaction. Our value of 1 indicates that the FLAP inhibitor Compound 2-94 and the NO-donor isosorbide mononitrate have an additive interaction.

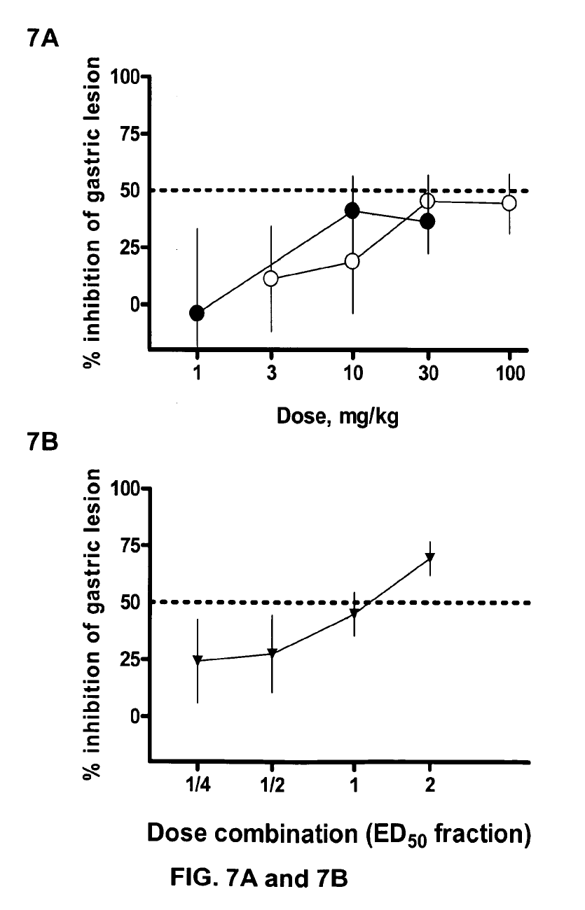

FIG. 7A. shows the close dependent inhibition of naproxen induced lesions using Compound 2-94 (filled circles) and isosorbide mononitrate (open circles).

FIG. 7B. shows the inhibition of naproxen, induced lesions using fractional doses of Compound 2-94 and isosorbide mononitrate.

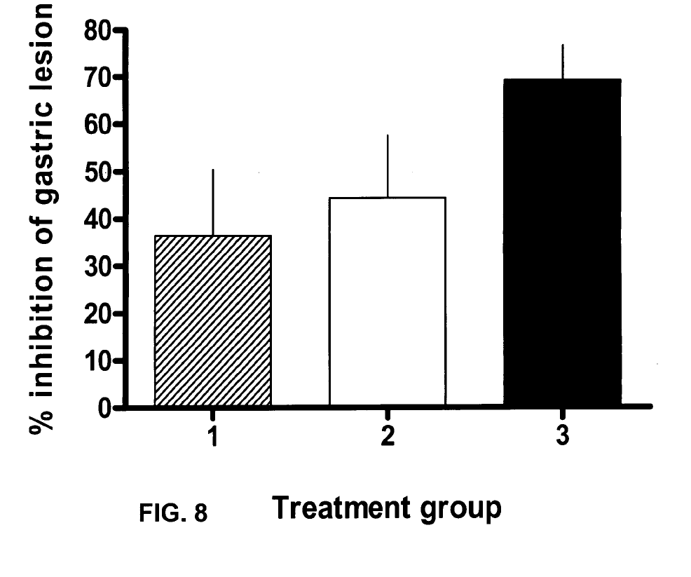

FIG. 8. shows the maximum inhibition achieved by Compound 2-94 alone (30 mg/kg, striped bar), isosorbide mononitrate alone (100 mg/kg, open bar) and by Compound 2-94 plus isosorbide mononitrate as a co-treatment (10 mg/kg+30 mg/kg, filled bar).

### Example 8

### Pharmaceutical Compositions

### Example 8a

### Parenteral Composition

To prepare a parenteral pharmaceutical composition suitable for administration by injection, 100 mg of a water-soluble salt of a compound of Formula (I) is dissolved in DMSO and then mixed with 10 mL of 0.9% sterile saline. The mixture is incorporated into a dosage unit form suitable for administration by injection.

### Example 8b

### Oral Composition

To prepare a pharmaceutical composition for oral delivery, 100 mg of a compound of Formula (I) is mixed with 750 mg of starch. The mixture is incorporated into an oral dosage unit for, such as a hard gelatin capsule, which is suitable for oral administration.

### Example 8c

### Sublingual (Hard Lozenge) Composition

To prepare a pharmaceutical composition for buccal delivery, such as a hard lozenge, mix 100 mg of a compound of Formula (I), with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 8d

### Inhalation Composition

To prepare a pharmaceutical composition for inhalation delivery, 20 mg of a compound of Formula (I) is mixed with 50 mg of anhydrous citric acid and 100 mL of 0.9% sodium chloride solution. The mixture is incorporated into an inhalation delivery unit, such as a nebulizer, which is suitable for inhalation administration.

### Example 8e

### Rectal Gel Composition

To prepare a pharmaceutical composition for rectal delivery, 100 mg of a compound of Formula (I) is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 8f

### Topical Gel Composition

To prepare a pharmaceutical topical gel composition, 100 mg of a compound of Formula (I) is mixed with 1.75 g of hydroxy-propyl celluose, 10 mL of propylene glycol, 10 mL of isopropyl myristate and 100 mL of purified alcohol USP. The resulting gel mixture is then incorporated into containers, such as tubes, which are suitable for topical administration.

### Example 8g

### Ophthalmic Solution Composition

To prepare a pharmaceutical ophthalmic solution composition, 100 mg of a compound of Formula (I) is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

### Example 9

### Combination Compounds of Formula (I)

Non-limiting examples of compounds of Formula (I) are presented in FIGS. 4-6. Such compound can be prepared using the methods described herein.

The examples and embodiments described herein are for illustrative purposes only and various modifications or changes suggested to persons skilled in the art are to be included within the spirit and purview of this application and scope of the appended claims. All publications, patents, and patent applications cited herein are hereby incorporated by reference for all purposes.

1. A pharmaceutical composition comprising an NO modulator, a FLAP inhibitor, and at least one pharmaceutically acceptable inactive ingredient selected from pharmaceutically acceptable diluents, pharmaceutically acceptable excipients, and pharmaceutically acceptable carriers.

2. The pharmaceutical composition as claimed in claim 1, wherein the pharmaceutical composition is an intravenous pharmaceutical composition, oral pharmaceutical composition, rectal pharmaceutical composition, aerosol pharmaceutical composition, parenteral pharmaceutical composition, ophthalmic pharmaceutical composition, pulmonary pharmaceutical composition, transmucosal pharmaceutical composition, transdermal pharmaceutical composition, vaginal pharmaceutical composition, otic pharmaceutical composition, nasal pharmaceutical composition, and topical pharmaceutical composition.

3. The pharmaceutical composition as claimed in claim 2, wherein the pharmaceutical composition is formulated for parenteral delivery, oral delivery, or intranasal delivery.

4. The pharmaceutical composition of claim 1, wherein the NO modulator is selected from nitroprusside, nitroglycerin, isosorbid mononitrate, isosorbid dinitrate, arginine, homoarginine, N-hydroxy-arginine, nitrosated arginine, nitrosylated arginine, nitrosated N-hydroxy-arginine, nitrosylated N-hydroxy-arginine, nitrosated homoarginine, nitrosylated homoarginine, citrulline, ornithine, glutamine, lysine, N-hydroxy-L-arginine, 2(S)-amino-6-boronohexanoic acid, adenosine, bradykinin, calreticulin, bisacodyl, phenolphthalein, molsidomine, 3-morpholinosydnonimine (SIN-1), 1,2,3,4-oxatriazolium, 5-amino-3-(3,4-di-chlorophenyl)-chloride (GEA 3162), 1,2,3,4-oxatriazolium, 5-amino-3-(3-chloro-2-methyl-phenyl)chloride (GEA502-4), 1,2,3,4-oxatriazolium, 3-(3-chloro-2-methylphenyl)-5-[[[cyanomethylamino-]carbonyl]amino]-hydroxide inner salt (GEA 5583); S-nitroso-N-acetyl-D,L-penicillamine (SNAP); Glyco-SNAP-1; Glyco-SNAP-2,2,2′-(hydroxynitrosohydrazono)bis-ethanamine (NOC-18) and (+/−)-(E)-4-ethyl-2-[(E)-hydroxyimino]-5-nitro-3-hexenamide (nor-3); 1-[(4′,5′-bis(carboxymethoxy)-2′-nitrophenyl)methoxy]-2-oxo-3,3,diethyl-1-triazene dipotassium salt (CNO-4); [1-(4′,5′-bis(carbomethoxy)-2′-nitrophenyl)methoxy]-2-oxo-3,3-diethyl-1-triazine diacetoxymethyl ester (CNO-5); b diethylamine-NO (DEA/NO), IPA/NO, spermine-NO (SPER/NO), sulfite-NO (SULFI/NO), OXI/NO, DETA/NO; cicletanine; GEA 3268, (1,2,3,4-oxatriazolium, 3-(3-chloro-2-methylphenyl)-5-[[(4-methoxyphenyl)-sulfonyl]amino]-, hydroxide inner salt); GEA 5145, (1,2,3,4-oxatriazolium, 3-(3-chloro-2-methylphenyl)-5-[(methylsulfonyl)amino]-, hydroxide inner salt); sulfonamide GEA 3175; S-nitrosothiols; nitrites; nitrates, N-oxo-N-nitrosoamines, SPM 3672, SPM 5185, and SPM 5186.

5. The pharmaceutical composition of claim 1, wherein the FLAP inhibitor has the structure of the FLAP compound (i): 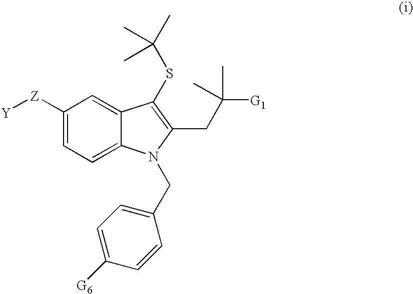
wherein,
Z is [C(R₂)₂]ₙC(R₁)₂O, wherein
each R₁ is independently H, —CF₃, or an optionally substituted lower alkyl; or two R₁ groups on the same carbon may join to form an oxo (═O); each R₂ is independently H, —OH, —OMe, —CF₃, or an optionally substituted lower alkyl; or two R₂ groups on the same carbon may join to form an oxo (═O); n is 0, 1, 2, or 3;
Y is -(substituted or unsubstituted aryl); or -(substituted or unsubstituted heteroaryl);
where each substituent on Y or Z is LₛRₛ, wherein each Lₛ is independently selected from a bond, —O—, —C(═O)—, —S(═O)—, —S(═O)₂—, —NHC(O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, substituted or unsubstituted C₁-C₆ alkyl, C₂-C₆ alkenyl, —C₁-C₆ fluoroalkyl, substituted or unsubstituted heteroaryl, substituted or unsubstituted aryl, or substituted or unsubstituted heterocycloalkyl; and each Rₛ is independently selected from H, halogen, —N(R₄)₂, —CN, —NO₂, N₃, —S(═O)₂NH₂, substituted or unsubstituted lower alkyl, substituted or unsubstituted lower cycloalkyl, —C₁-C₆ fluoroalkyl, substituted or unsubstituted aryl, substituted or unsubstituted heteroaryl, or substituted or unsubstituted heteroalkyl;
G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—;
or G₁ is W-G₅, where W is a substituted or unsubstituted aryl, substituted or unsubstituted heterocycloalkyl or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, —CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —CON(R₉)₂, —SR₈, —S(═O)R₈, or —S(═O)₂R₈;
each R₈ is independently selected from substituted or unsubstituted lower alkyl, substituted or unsubstituted lower cycloalkyl, substituted or unsubstituted phenyl or substituted or unsubstituted benzyl;
each R₉ is independently selected from h, substituted or unsubstituted lower alkyl, substituted or unsubstituted lower cycloalkyl, substituted or unsubstituted phenyl or substituted or unsubstituted benzyl; or
two R₉ groups can together form a 5-, 6-, 7-, or 8-membered heterocyclic ring; or
R₈ and R₉ can together form a 5-, 6-, 7-, or 8-membered heterocyclic ring;
each R₁₀ is independently selected from H, —S(═O)₂R₈, —S(═O)₂NH₂—C(O)R₈, —CN, —NO₂, heteroaryl, or heteroalkyl;
G₆ is W-G₇, wherein W is (substituted or unsubstituted heterocycloalkyl), (substituted or unsubstituted aryl) or a (substituted or unsubstituted heteroaryl); and
G₇ is H, halogen, CN, NO₂, N₃, CF₃, OCF₃, C₁-C₆ alkyl, C₁-C₆ heteroalkyl C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is a bond, —O—, C(═O)—, —S—, —S(═O)—, —S(═O)—, —NH—, —NHC(═O)O—, —NHC(═O)NH—, —OC(═O)O—, —OC(═O)NH—, —NHC(═O)—, —C(═O)NH—, —C(═O)O—, or —OC(═O)—; or solvate, or pharmaceutically acceptable salt, or a pharmaceutically acceptable prodrug thereof.

6. The pharmaceutical composition as claimed in claim 1, wherein the NO modulator and FLAP modulator are chemically linked.

7. The pharmaceutical composition as claimed in claim 6, wherein the NO modulator and the FLAP modulator are chemically linked to form a compound of Formula (I):
Aₓ-L-B Formula (I)
wherein,
A is:
a) a moiety that in the form A′ is an NO-modulator, wherein A′ is A-X, A-H, A⁻, or A⁺; X is COOH, CONH₂, OH, NH₂, halogen, SH, or CH₃;
b) a moiety that upon activation/reaction produces NO; or
c) a moiety selected from —NO₂ and —ONO₂;
x is 0, 1, 2, 3, 4, 5, 6, 7, 8, 9, 10, 11, 12, 13, 14, 15, 16, 17, 17, 19, or 20;
L is a bond or a moiety that chemically links the A and B moieties, and is cleaved in a single step or in multiple steps by chemical, enzymatical, biological, photochemical, electrochemical, or sonochemical means in vivo to produce -L′-A and -B′, -L′-B and -A′, or -L′, -A′ and -B′;
B is a moiety that in the form B′ is a FLAP inhibitor, wherein B′ is B-X, B-H, B⁻, or B⁺; X is —CO₂H, —CONH₂, —OH, —NH₂, halogen, —SH, —CH₃ or —CH₂OH.

8. The pharmaceutical composition of claim 6, wherein the NO modulator is an NO releaser or NO inducer.

9. A method of treating NSAID-induced gastric lesions or hypertension in a human comprising administering to the human the pharmaceutical composition of claim 1.

10. A method for treating a patient having a disease, disorder or condition wherein at least one symptom results from an NO level in the patient comprising administering a pharmaceutical composition of claim 1 to the patient, wherein the FLAP inhibitor and the NO modulator are administered separately in time or simultaneously.

11. (canceled)

12. (canceled)

13. A method for ameliorating or eliminating at least one adverse effect arising from administering an NO modulator to a patient, wherein the at least one adverse effect is leukotriene-dependent or leukotriene-modulated, comprising administering a pharmaceutical composition of claim 1 to the patient, wherein the FLAP inhibitor is administered prior to, simultaneous with, or after administration of the NO modulator.

14. (canceled)

15. (canceled)

16. A method for ameliorating or eliminating at least one adverse effects of leukotriene therapy in a patient, wherein the at least one adverse effect is NO-dependent or NO-modulated, comprising administering a pharmaceutical composition of claim 1 to the patient, wherein the NO modulator is administered prior to, simultaneous with, or after administration of the FLAP inhibitor.

17. (canceled)

18. (canceled)

19. (canceled)

20. A method for improving the effectiveness of a bronchodilator in a patient comprising administering a pharmaceutical composition of claim 1 to the patient, wherein the FLAP inhibitor and NO modulator is administered prior to, simultaneous with, or after administration of the bronchodilator.

21. (canceled)

22. (canceled)

23. A method for improving the effectiveness of an anti-inflammatory in a patient, wherein the anti-inflammatory is selected from one or more natural/synthetic/semi-synthetic glucocorticoids, leukotriene synthesis inhibitors, leukotriene receptor blockers, antihistamines, comprising administering a pharmaceutical composition of claim 1 to the patient, wherein the FLAP inhibitor and NO modulator is administered prior to, simultaneous with, or after administration of the anti-inflammatory.

24. (canceled)

25. (canceled)

26. A method for improving the effectiveness of antibiotics in a patient with a pulmonary or vascular infection and a clinical condition selected from the group consisting of asthma, COPD, cystic fibrosis, pneumonia, traumatic injury, aspiration or inhalation injury, fat embolism in the lung, acidosis, inflammation of the lung, adult respiratory distress syndrome, acute mountain sickness, post cardiac surgery acute pulmonary hypertension, persistent pulmonary hypertension of the newborn, perinatal aspiration syndrome, hyaline membrane disease, acute pulmonary thromboembolism, acute pulmonary edema, heparin-protamine reactions, sepsis, hypoxia, comprising administering a pharmaceutical composition of claim 1 to the patient, wherein the FLAP inhibitor and NO modulator is administered prior to, simultaneous with, or after administration of the antibiotic.

27. (canceled)

28. (canceled)

29. The method of claim 26, wherein the antibiotic is chosen from one or more of the following: antibacterials, antifungals, antiprotozoals, or antivirals.

30. The method of claim 29 where the antibacterial is chosen from one or more of the following: natural/synthetic/semi-synthetic aminoglycosides, beta-lactams, cephalosporins, immunomodulators or immunostimulants, ketolides, macrolides, oxazolidiones, penicillins, quinolones, teicoplanin or clindamycin or streptomycin or vancomycin or oritovancin, and deformylase inhibitors, and ribosome inhibitors, and quorum sensing inhibitors, or analogs thereof.

31. A method for treating a patient having a disease, disorder or condition wherein at least one symptom results from the activity of 5-lipoxygenase activating protein (FLAP) comprising administering to the patient a pharmaceutical composition of claim 1, wherein the FLAP inhibitor and the NO modulator are administered separately in time or simultaneously.

32. (canceled)

33. (canceled)

34. A compound having the structure of Formula (I):
Aₓ-L-B Formula (I)
wherein,
A is:
a) a moiety that in the form A′ is an NO-modulator, wherein A′ is A-X, A-H, A⁻, or A⁺; X is COOH, CONH₂, OH, NH₂, halogen, SH, or CH₃;
b) a moiety that upon activation/reaction produces NO; or
c) a moiety selected from —NO₂ and —ONO₂;
x is 0, 1, 2, 3, 4, 5, 6, 7, 8, 9, 10, 11, 12, 13, 14, 15, 16, 17, 17, 19, or 20;
L is a bond or a moiety that chemically links the A and B moieties, and is cleaved in a single step or in multiple steps by chemical, enzymatical, biological, photochemical, electrochemical, or sonochemical means in vivo to produce -L′-A and -B′, -L′-B and -A′, or -L′, -A′ and -B′;
B is a moiety that in the form B′ is a FLAP inhibitor, wherein B′ is B-X, B-H, B⁻, or B⁺; X is —CO₂H, —CONH₂, —OH, —NH₂, halogen, —SH, —CH₃ or —CH₂OH;
wherein:
A′ is selected from nitroprusside, nitroglycerin, isosorbid mononitrate, isosorbid dinitrate, arginine, homoarginine, N-hydroxy-arginine, nitrosated arginine, nitrosylated arginine, nitrosated N-hydroxy-arginine, nitrosylated N-hydroxy-arginine, nitrosated homoarginine, nitrosylated homoarginine, citrulline, ornithine, glutamine, lysine, N-hydroxy-L-arginine, 2(S)-amino-6-boronohexanoic acid, adenosine, bradykinin, calreticulin, bisacodyl, phenolphthalein, molsidomine, 3-morpholinosydnonimine (SIN-1), 1,2,3,4-oxatriazolium, 5-amino-3-(3,4-dichlorophenyl)-chloride (GEA 3162), 1,2,3,4-oxatriazolium, 5-amino-3-(3-chloro-2-methylphenyl)chloride (GEA502-4), 1,2,3,4-oxatriazolium, 3-(3-chloro-2-methylphenyl)-5-[[[cyanomethylamino-]carbonyl]amino]-hydroxide inner salt (GEA 5583); S-nitroso-N-acetyl-D,L-penicillamine (SNAP); Glyco-SNAP-1; Glyco-SNAP-2,2,2′-(hydroxynitrosohydrazono)bis-ethanamine (NOC-18) and (+/−)-(E)-4-ethyl-2-[(E)-hydroxyimino]-5-nitro-3-hexenamide (NOR-3); 1-[(4′,5′-bis(carboxymethoxy)-2′-nitrophenyl)methoxy]-2-oxo-3,3,diethyl-1-triazene dipotassium salt (CNO-4); [1-(4′,5′-bis(carbomethoxy)-2′-nitrophenyl)methoxy]-2-oxo-3,3-diethyl-1-triazine diacetoxymethyl ester (CNO-5); b diethylamine-NO (DEA/NO), IPA/NO, spermine-NO(SPER/NO), sulfite-NO (SULFI/NO), OXI/NO, DETA/NO; cicletanine; GEA 3268, (1,2,3,4-oxatriazolium, 3-(3-chloro-2-methylphenyl)-5-[[(4-methoxyphenyl)-sulfonyl]amino]-, hydroxide inner salt); GEA 5145, (1,2,3,4-oxatriazolium, 3-(3-chloro-2-methylphenyl)-5-[(methylsulfonyl)amino]-, hydroxide inner salt); sulfonamide GEA 3175; S-nitrosothiols; nitrites; nitrates, N-oxo-N-nitrosoamines, SPM 3672, SPM 5185, and SPM 5186; and
B′ has the structure of FLAP compound (i): 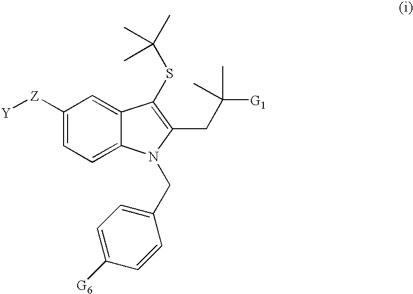
wherein,
Z is [C(R₂)₂]ₙC(R₁)₂O, wherein
each R₁ is independently H, —CF₃, or an optionally substituted lower alkyl; or two R₁ groups on the same carbon may join to form an oxo (═O); each R₂ is independently H, —OH, —OMe, —CF₃, or an optionally substituted lower alkyl; or two R₂ groups on the same carbon may join to form an oxo (═O); n is 0, 1, 2, or 3;
Y is -(substituted or unsubstituted aryl); or -(substituted or unsubstituted heteroaryl);
where each substituent on Y or Z is LₛRₛ, wherein each Lₛ is independently selected from a bond, —O—, —C(═O)—, —S—, —S(═O)—, —S(═O)₂—, —NHC(O)—, —C(═O)NH—, S(═O)₂NH—, —NHS(═O)₂, —OC(═O)NH—, —NHC(═O)O—, —OC(═O)O—, —NHC(═O)NH—, —C(═O)O—, —OC(═O)—, substituted or unsubstituted C₁-C₆ alkyl, C₂-C₆ alkenyl, —C₁-C₆ fluoroalkyl, substituted or unsubstituted heteroaryl, substituted or unsubstituted aryl, or substituted or unsubstituted heterocycloalkyl; and
each Rₛ is independently selected from H, halogen, —N(R₄)₂, —CN, —NO₂, N₃, —S(═O)₂NH₂, substituted or unsubstituted lower alkyl, substituted or unsubstituted lower cycloalkyl, —C₁-C₆ fluoroalkyl, substituted or unsubstituted aryl, substituted or unsubstituted heteroaryl, or substituted or unsubstituted heteroalkyl;
G₁ is H, tetrazolyl, —NHS(═O)₂R₈, S(═O)₂N(R₉)₂, —OR₉, —C(═O)CF₃, —C(O)NHS(═O)₂R₈, —S(═O)₂NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(O)R₉, —CON(R₉)₂, —SR₈, —S(═O)R₈, —S(═O)₂R₈, -L₅-(substituted or unsubstituted alkyl), -L₅-(substituted or unsubstituted alkenyl), -L₅-(substituted or unsubstituted heteroaryl), or -L₅-(substituted or unsubstituted aryl), wherein L₅ is —OC(═O)O—, —NHC(═O)NH—, —NHC(═O)O—, —OC(═O)NH—, —NHC(═O)—, —C(═O)NH—, —C(═O)O—, or —OC(═O)—;
or G₁ is W-G₅, where W is a substituted or unsubstituted aryl, substituted or unsubstituted heterocycloalkyl or substituted or unsubstituted heteroaryl and G₅ is H, tetrazolyl, —NHS(═O)₂R₈, S(═O)₂N(R₉)₂, OH, —OR₈, —C(═O)CF₃, —C(O)NHS(═O)₂R₈, —S(═O)₂NHC(O)R₉, —CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —CON(R₉)₂, —SR₈, —S(═O)R₈, or —S(═O)₂R₈;
each R₈ is independently selected from substituted or unsubstituted lower alkyl, substituted or unsubstituted lower cycloalkyl, substituted or unsubstituted phenyl or substituted or unsubstituted benzyl;
each R₉ is independently selected from H, substituted or unsubstituted lower alkyl, substituted or unsubstituted lower cycloalkyl, substituted or unsubstituted phenyl or substituted or unsubstituted benzyl; or
two R₉ groups can together form a 5-, 6-, 7-, or 8-membered heterocyclic ring; or
R₈ and R₉ can together form a 5-, 6-, 7-, or 8-membered heterocyclic ring;
each R₁₀ is independently selected from H, —S(═O)₂R₈, —S(═O)₂NH₂—C(O)R₈, —CN, —NO₂, heteroaryl, or heteroalkyl;
G₆ is W-G₇, wherein W is (substituted or unsubstituted heterocycloalkyl), (substituted or unsubstituted aryl) or a (substituted or unsubstituted heteroaryl); and
G₇ is H, halogen, CN, NO₂, N₃, CF₃, OCF₃, C₁-C₆ alkyl, C₁-C₆ heteroalkyl, C₃-C₆ cycloalkyl, —C₁-C₆ fluoroalkyl, tetrazolyl, —NHS(═O)₂R₈, S(═O)₂N(R₉)₂, OH, —OR₈, —C(═O)CF₃, —C(O)NHS(═O)₂R₈, —S(═O)NHC(O)R₉, CN, N(R₉)₂, —N(R₉)C(O)R₉, —C(═NR₁₀)N(R₉)₂, —NR₉C(═NR₁₀)N(R₉)₂, —NR₉C(═CHR₁₀)N(R₉)₂, —C(O)NR₉C(═NR₁₀)N(R₉)₂, —C(O)NR₉C(═CHR₁₀)N(R₉)₂, —CO₂R₉, —C(═O)R₉, —CON(R₉)₂, —SR₈, —S(═O)R₈, or —S(═O)₂R₈, -L₅-(substituted or unsubstituted alkyl), -L₅-(substituted or unsubstituted alkenyl), -L₅-(substituted or unsubstituted heteroalkyl), -L₅-(substituted or unsubstituted heteroaryl), -L₅-(substituted or unsubstituted heterocycloalkyl), or -L₅-(substituted or unsubstituted aryl), wherein L₅ is a bond, —O—, C(═O)—, —S—, —S(═O)—, —S(═O)₂—, —NH—, —NHC(═O)O—, —NHC(═O)NH—, —OC(═O)O—, —OC(═O)NH—, —NHC(═O)—, —C(═O)NH—, —C(═O)O—, or —OC(═O)—;
or solvate, or pharmaceutically acceptable salt, or a pharmaceutically acceptable prodrug thereof.

35-60. (canceled)
